(19) United States
(12) Patent Application Publication
(10) Pub. No.: US 2024/0294989 A1
(43) Pub. Date: Sep. 5, 2024

(54) COMPOSITIONS AND METHODS FOR SCREENING SOLID TUMORS

(71) Applicant: Quest Diagnostics Investments LLC, Secaucus, NJ (US)

(72) Inventors: Heather SANDERS, San Juan Capistrano, CA (US); Kevin QU, San Juan Capistrano, CA (US); James PRENTICE, San Juan Capistrano, CA (US); Frederic WALDMAN, San Juan Capistrano, CA (US)

(73) Assignee: Quest Diagnostics Investments LLC, Secaucus, NJ (US)

(21) Appl. No.: 18/662,594

(22) Filed: May 13, 2024

Related U.S. Application Data
(62) Division of application No. 16/870,033, filed on May 8, 2020, now Pat. No. 11,981,966.
Application No. 16/870,033 is a continuation of application No. 15/576,219, filed on Nov. 21, 2017, now Pat. No. 10,689,710.
(60) Provisional application No. 62/166,996, filed on May 27, 2015.
(60) Provisional application No. 62/246,895, filed on Oct. 27, 2015.

(51) Int. Cl. C12Q 1/6886

(52) U.S. Cl. CPC C12Q 1/6886; C12Q 2600/106; C12Q 2600/156; C12Q 2600/16

(57) ABSTRACT

The present technology relates to methods for determining whether a patient diagnosed with breast cancer, colorectal cancer, melanoma or lung cancer will benefit from or is predicted to be responsive to treatment with an individual therapeutic agent or a specific combination of therapeutic agents. These methods are based on screening a patient's solid tumors and detecting alterations in target nucleic acid sequences corresponding to a specific set of cancer-related genes. Kits for use in practicing the methods are also provided.

### CROSS-REFERENCE TO RELATED APPLICATIONS

This application is a Divisional of U.S. application Ser. No. 16/870,033, filed May 8, 2020, which is a Continuation of U.S. application Ser. No. 15/576,219, which is the U.S. National Stage Application of PCT/US2015/057733, filed Oct. 28, 2015 which claims the benefit of and priority to U.S. Application No. 62/166,996, filed May 27, 2015, and to U.S. Application No. 62/246,895, filed Oct. 27, 2015, the contents of which are incorporated herein by reference in their entireties.

### SEQUENCE LISTING

The instant application contains a Sequence Listing which has been submitted in XML format and is hereby incorporated by reference in its entirety. Said XML copy, created on May 2, 2024, is named 034827-1988_SL.xml and is 602,826 bytes.

### TECHNICAL FIELD

The present technology relates to methods for determining whether a patient diagnosed with breast cancer, colorectal cancer, melanoma or lung cancer will benefit from or is predicted to be responsive to treatment with a therapeutic agent alone or in a specific combination with other therapeutic agents. These methods are based on screening a patient's solid tumors and detecting alterations in target nucleic acid sequences corresponding to a specific set of cancer-related genes. Kits for use in practicing the methods are also provided.

### BACKGROUND

The following description of the background of the present technology is provided simply as an aid in understanding the present technology and is not admitted to describe or constitute prior art to the present technology.

The development of companion diagnostics has the potential to improve patient outcomes, and eliminate the need for insurers to pay for expensive yet ineffective therapies. As the number of genes implicated in cancer continues to grow, it is becoming evident that a careful characterization of the genetic alterations that define an individual patient's tumors will often be useful in determining optimal therapeutic strategies. For example, Foundation Medicine® offers a large-scale solid tumor screening panel that interrogates the entire coding sequence of over 300 cancer-related genes and 28 gene rearrangements using DNA or RNA bait libraries (e.g., FoundationOne®). Such assays require a tumor DNA input of at least 50 ng. However, the amount of DNA available for such comprehensive studies is often limited and of poor quality because the tumor DNA is isolated from formalin fixed paraffin-embedded (FFPE) tissues. The FFPE process frequently degrades DNA into small fragments and has the potential to damage the DNA base pairs themselves.

TruSight™ Tumor (Illumina, Inc.) is an example of an existing PCR-based NGS tumor screening panel that interrogates a narrower set of cancer-related genes (174 amplicons within 26 genes) and requires a minimum DNA input of 30 ng. However, this method requires an evaluation of the quality of genomic DNA extracted from the FFPE tumor sample via quantitative PCR prior to generating an amplicon-based library because neither tissue area nor DNA yield are adequate predictors of library performance. See TruSight™ Tumor Data Sheet (Illumina, Inc.); Generating Sequencing Libraries from FFPE Samples, White Paper (Illumina, Inc.).

Detecting actionable genetic alterations in FFPE tissues is further complicated by the fact that cells within a tumor sample can exhibit a high degree of molecular variation between tumors (inter-tumor heterogeneity) and within the individual tumor itself (intra-tumor heterogeneity). Tumor heterogeneity has been observed in leukemias, melanomas, breast, prostate, colon, lung, and gynecological carcinomas. Accordingly, the small fraction of cells in a biopsy may not be representative of the entire tumor mass, which could lead to false negative calls for a given genetic alteration.

Intra-tumor heterogeneity may also explain, at least in part, why some patients who initially respond well to a cancer drug eventually relapse, often with new tumors that no longer respond to the therapy. The higher the diversity of cells within a tumor, the greater the risk that an occasional cell might be able to adapt to the type of stress a drug imposes. Acquired resistance to cancer drugs may develop through a variety of mechanisms (Chong C. & Jänne P., Nat. Med. 19: 1389-1400 (2013); Katayama et al., Sci. Transl. Med. 4(120): 120ra17 (2012)). For example, resistant cells can develop a compensatory signaling pathway, or “bypass track,” that reestablishes activation of key downstream proliferation and survival signals despite inhibition of the original oncogene (Niederst & Engelman, Sci. Signal. 6: re6 (2013)). Thus, the heterogeneity of cancer cells introduces significant challenges in designing effective treatment strategies, especially when a specific gene mutation is not detected in the biopsy.

Thus, there is a substantial need for more robust and sensitive methods that effectively detect the presence of genetic alterations in highly heterogeneous tumors samples, particularly in FFPE tissues. Such methods would aid in predicting the responsiveness of individual patients to a particular drug regimen and the identification of optimal therapeutic strategies at the outset.

### SUMMARY OF THE PRESENT TECHNOLOGY

The methods and compositions disclosed herein relate to the detection of mutations that are predictive of the responsiveness of a subject diagnosed with breast cancer, colorectal cancer, melanoma, or lung cancer to a particular therapeutic regimen. In another aspect, the methods and compositions of the present technology are useful in selecting or designing an optimal therapeutic regimen for a subject diagnosed with breast cancer, colorectal cancer, melanoma, or lung cancer. It is contemplated that the methods disclosed herein allow for rapid and sensitive detection of mutations in the target nucleic acid sequences of AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, 00, EGFR, FGFR4, IDH1, NOTCH1, and PTEN. In some embodiments, the therapeutic regimen comprises one or more of anti-HER-2 therapies, P13K/AKT/mTor pathway inhibitors, receptor tyrosine kinase inhibitors (TKIs), Notch pathway inhibitors, BRAF inhibitors, SMO antagonists, ALK/MET inhibitors, ERBB2 antagonists, FGFR3 antagonists, and RAF/MEK/ERK inhibitors.

In one aspect, the present disclosure provides a method for detecting at least one mutation in a plurality of cancer-related genes in a subject comprising (a) extracting genomic DNA from a formalin fixed paraffin-embedded tumor sample obtained from the subject; (b) generating a library comprising amplicons corresponding to each of the plurality of cancer-related genes, said plurality of cancer-related genes comprising AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN, wherein (i) generating said library occurs without the use of a bait set comprising nucleic acid sequences that are complementary to at least one of the plurality of amplicons; and (ii) the quality of the genomic DNA extracted from the formalin fixed paraffin-embedded tumor sample is not assessed using quantitative PCR prior to generating the library; (c) ligating an adapter sequence to the ends of the plurality of amplicons; and (d) detecting at least one mutation in at least one of the plurality of amplicons using high throughput massive parallel sequencing.

In some embodiments of the method, the plurality of amplicons is generated by at least two primer pairs disclosed in Table 1, Table 2, or a combination thereof.

In some embodiments of the method, the at least one mutation detected is a mutation in EGFR, KRAS, BRAF, NRAS, ERBB2 or PIK3CA. In one embodiment, the at least one mutation detected is selected from the group consisting of BRAF V600E, BRAF V600K, BRAF K483Q, BRAF G466V, BRAF G464V, BRAF E501V, BRAF E501K, EGFR ΔE746_A750, EGFR R680Q, EGFR G598E, KRAS A146T, KRAS R68M, KRAS L19F, KRAS G12V, KRAS G12D, KRAS G12C, KRAS G13D, KRAS G13C, KRAS G12A, KRAS G12S, KRAS Q22K, NRAS Q61K, NRAS Q61R, NRAS G12R, NRAS G12D, PIK3CA C420R, PIK3CA G106R, PIK3CA R38H, PIK3CA E453K, PIK3CA H1044R, PIK3CA N1044K, PIK3CA E545K, PIK3CA Q546H, PIK3CA H1047R, PIK3CA H1043L, PIK3CA M1043V, PIK3CA E542K, PIK3CA E542Q, PIK3CA T1053A, PIK3CA 1121V, PIK3CA H1047L, ERBB2 L755S, ERBB2 S310Y, ERBB2 D769Y, ERBB2 S255R, DDR2 H92Y, DDR2 R31L, DDR2 L34P, DDR2 P381R and DDR2 K392N.

In some embodiments of the method, the library comprising amplicons corresponding to each of the plurality of cancer-related genes is generated using no more than 10 ng of extracted genomic DNA from the formalin fixed paraffin-embedded tumor sample.

In some embodiments of the method, the library comprising amplicons corresponding to each of the plurality of cancer-related genes is generated using 11-25 ng of extracted genomic DNA from the formalin fixed paraffin-embedded tumor sample.

In certain embodiments, the high throughput massive parallel sequencing is performed using pyrosequencing, reversible dye-terminator sequencing, SOLID sequencing, Ion semiconductor sequencing, Helioscope single molecule sequencing, sequencing by synthesis, sequencing by ligation, or SMRT™ sequencing.

In some embodiments of the method, the adapter sequence is a P5 adapter, P7 adapter, P1 adapter, A adapter, or Ion Xpress™ barcode adapter.

Additionally or alternatively, in some embodiments, the plurality of amplicons further comprises a unique index sequence.

In some embodiments, the formalin fixed paraffin-embedded tumor sample is a heterogeneous tumor. In certain embodiments, 5% of the cells of the heterogeneous tumor harbor at least one mutation in at least one of the plurality of amplicons.

In some embodiments of the method, the subject has been diagnosed with breast cancer, melanoma, colorectal cancer or lung cancer.

In another aspect, the present disclosure provides a method for selecting a subject for treatment with a P13K/AKT/mTor pathway inhibitor and at least one additional agent comprising (a) extracting genomic DNA from a formalin fixed paraffin-embedded specimen obtained from the subject; (b) generating a library comprising amplicons corresponding to each of a plurality of cancer-related genes, said plurality of cancer-related genes comprising AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN, wherein (i) generating said library occurs without the use of a bait set comprising nucleic acid sequences that are complementary to at least one of the plurality of amplicons, and (ii) the quality of the genomic DNA extracted from the formalin fixed paraffin-embedded specimen is not assessed using quantitative PCR prior to generating the library; (c) detecting at least one mutation in at least one of the plurality of amplicons; and (d) selecting the subject for treatment with a P13K/AKT/mTor pathway inhibitor and at least one additional agent, if a mutation in at least one of the amplicons corresponding to PIK3CA, PIK3R1 and PTEN, and a mutation in at least one of the amplicons corresponding to NOTCH1, ERBB2, BRAF, PTCH1, SMO, EGFR, KRAS, DDR2, MAP2K1, FGFR3, NRAS, MET, and FBXW7 are detected.

In some embodiments of the method, the amplicons corresponding to PIK3CA are generated by a pair of primers selected from the group consisting of 5′ CCTAGTAGAATGTTTACTACCAA 3′ (SEQ ID NO.: 1) and 5′ CTGCTTCTTGAGTAACACTT 3′ (SEQ ID NO.: 2); 5′ CATGTTCATGCTGTGTATGT 3′ (SEQ ID NO.: 3) and 5′ GCTTCTTTACAAACGTTCAGAA 3′ (SEQ ID NO.: 4); 5′ TCTATGTTCGAACAGGTATCT 3′ (SEQ ID NO.: 5) and 5′ ACTGCTAAACACTAATATAACCTTTG 3′ (SEQ ID NO.: 6); 5′ TTGAAATGTGTTTTATAATTTAGACTAGT 3′ (SEQ ID NO.: 7) and 5′ CCATGAGGTACTGGCC 3′ (SEQ ID NO.: 8); 5′ TTGGTGTTACTGGATCAAATC 3′ (SEQ ID NO.: 9) and 5′ TGCTGAACCAGTCAAACT 3′ (SEQ ID NO.: 10); 5′ TATTATTTTATTTTACAGAGTAACAGACTAG 3′ (SEQ ID NO.: 11) and 5′ TTTAGCACTTACCTGTGACT 3′ (SEQ ID NO.: 12); 5′ TGGAATGCCAGAACTACA 3′ (SEQ ID NO.: 13) and 5′ GTGGAAGATCCAATCCATTTT 3′ (SEQ ID NO.: 14); 5′ GGAATGAATGGCTGAATTATG 3′ (SEQ ID NO.: 15) and 5′ GCGGTATAATCAGGAGTTTT 3′ (SEQ ID NO.: 16); 5′ AGTTGGCCTGAATCACTATA 3′ (SEQ ID NO.: 17) and 5′ GATGTTACTATTGTGACGATCTC 3′ (SEQ ID NO.: 18); 5′ GTAAGTGTTACTCAAGAAGC 3′ (SEQ ID NO.: 19) and 5′ ATAGGATATTGTATCATACCAATTTCT 3′ (SEQ ID NO.: 20); 5′ TCCACAGCTACACCATATAT 3′ (SEQ ID NO.: 21) and 5′ AGCATCAGCATTTGACTTTA 3′ (SEQ ID NO.: 22); 5′ TACACAGACACTCTAGTATCTG 3′ (SEQ ID NO.: 23) and 5′ GAAGGTTTGACTGCCATAAA 3′ (SEQ ID NO.: 24); 5′ ATGACAAAGAACAGCTCAAA 3′ (SEQ ID NO.: 25) and 5′ GAGATCAGCCAAATTCAGTT 3′ (SEQ ID NO.: 26); 5′ GATGTGTTACAAGGCTTATCTA 3′ (SEQ ID NO.: 27) and 5′ GCCTCTTGCTCAGTTTTATC 3′ (SEQ ID NO.: 28); 5′ GAGGCTTTGGAGTATTTCA 3′ (SEQ ID NO.: 29) and 5′ CTGCTGAGAGTTATTAACAGT 3′ (SEQ ID NO.: 30); and 5′ GCTTTTGGAGTCCTATTGT 3′ (SEQ ID NO.: 31) and 5′ CACAAACTAGAGTCACACAC 3′ (SEQ ID NO.: 32).

In some embodiments of the method, the amplicons corresponding to PIK3R1 are generated by a pair of primers selected from the group consisting of 5′ GGGTTTTGGGCTGATATTA 3′ (SEQ ID NO.: 33) and 5′ CCACAGAACTGAAGGTTAAT 3′ (SEQ ID NO.: 34); 5′ TTATCCATTGAATTTATTTTAATCTTTCTAG 3′ (SEQ ID NO.: 35) and 5′ GGGATGTGCGGGTATATT 3′ (SEQ ID NO.: 36); 5′ GTCTTGCAGTAAGAGATTGT 3′ (SEQ ID NO.: 37) and 5′ TCTTTGCTGTACCGCT 3′ (SEQ ID NO.: 38); 5′ GTTTCTTTTGCCTGCA 3′ (SEQ ID NO.: 39) and 5′ TGGATAAGGTCTGGTTTAATG 3′ (SEQ ID NO.: 40); 5′ GCTACAATTCAGGATGAGTTA 3′ (SEQ ID NO.: 41) and 5′ TCTTCTGCTATCACCATCTTT 3′ (SEQ ID NO.: 42); 5′ CCATCATGATGAGAAGACAT 3′ (SEQ ID NO.: 43) and 5′ TTGCTGGAGATACATACACT 3′ (SEQ ID NO.: 44); 5′ GTGGTCACTAAACCTTAAGA 3′ (SEQ ID NO.: 45) and 5′ GGCTTACCTTAGTGTAAGAG 3′ (SEQ ID NO.: 46); 5′ TTTCATCGAGATGGGAAATATG 3′ (SEQ ID NO.: 47) and 5′ ACCTGTTGGTATTTGGATACT 3′ (SEQ ID NO.: 48); 5′ AGAAGATAATATTGAAGCTGTAGG 3′ (SEQ ID NO.: 49) and 5′ AGAACTCTTATTTTTTAATCTGATTTTCA 3′ (SEQ ID NO.: 50); 5′ GGACAGCTATTGAAGCATTTA 3′ (SEQ ID NO.: 51) and 5′ CACAAGAACAAGGGAAACAC 3′ (SEQ ID NO.: 52); 5′ GCAGGCAGCTGAGTATC 3′ (SEQ ID NO.: 53) and 5′ TCATCCTGAATTGTAGCAATCA 3′ (SEQ ID NO.: 54).

In some embodiments of the method, the amplicons corresponding to PTEN are generated by a pair of primers selected from the group consisting of 5′ CAGCTTCTGCCATCTCT 3′ (SEQ ID NO.: 55) and 5′ AGCAGCCGCAGAAAT 3′ (SEQ ID NO.: 56); 5′ GTGGCTTTTTGTTTGTTTG 3′ (SEQ ID NO.: 57) and 5′ CACTCTAACAAGCAGATAACT 3′ (SEQ ID NO.: 58); 5′ TACTTGTTAATTAAAAATTCAAGAGTTTT 3′ (SEQ ID NO.: 59) and 5′ CTTAGCCATTGGTCAAGATC 3′ (SEQ ID NO.: 60); 5′ ACAATCATGTTGCAGCA 3′ (SEQ ID NO.: 61) and 5′ AAAAACATCAAAAAATAACTTACCTTTT 3′ (SEQ ID NO.: 62); 5′ AGAGGCGCTATGTGTATTA 3′ (SEQ ID NO.: 63) and 5′ CATGGAAGGATGAGAATTTCA 3′ (SEQ ID NO.: 64); 5′ GGAAGACAAGTTCATGTACT 3′ (SEQ ID NO.: 65) and 5′ CTGTCCTTATTTTGGATATTTCTC 3′ (SEQ ID NO.: 66); 5′ ATTAATTAAATATGTCATTTCATTTCTTTTTC 3′ (SEQ ID NO.: 67) and 5′ GCTATCGATTTCTTGATCACA 3′ (SEQ ID NO.: 68); 5′ TGAGTCATATTTGTGGGTTTTC 3′ (SEQ ID NO.: 69) and 5′ TGATCAGGTTCATTGTCACTAA 3′ (SEQ ID NO.: 70); 5′ TTTGATTGCTGCATATTTCAG 3′ (SEQ ID NO.: 71) and 5′ TCAAAGCATTCTTACCTTACTAC 3′ (SEQ ID NO.: 72); 5′ TTTTAAACTTTTCTTTTAGTTGTGC 3′ (SEQ ID NO.: 73) and 5′ ACTCGATAATCTGGATGACT 3′ (SEQ ID NO.: 74); 5′ CAATTTAGTGAAATAACTATAATGGAAC 3′ (SEQ ID NO.: 75) and 5′ AGTGCCACTGGTCTATAAT 3′ (SEQ ID NO.: 76); 5′ CCTGTGAAATAATACTGGTATGT 3′ (SEQ ID NO.: 77) and 5′ CTACTTTGATATCACCACACAC 3′ (SEQ ID NO.: 78); 5′ TAGAGCGTGCAGATAATGA 3′ (SEQ ID NO.: 79) and 5′ TCAACAACCCCCACAAA 3′ (SEQ ID NO.: 80); and 5′ CTTTCTCTAGGTGAAGCTGTA 3′ (SEQ ID NO.: 81) and 5′ GGTTCATTCTCTGGATCAGA 3′ (SEQ ID NO.: 82).

In some embodiments of the method, the formalin fixed paraffin-embedded specimen is a heterogeneous tumor. In certain embodiments, 5%-10% of the cells of the heterogeneous tumor harbor at least one mutation in at least one of the plurality of amplicons. In other embodiments, at least 10% of the cells of the heterogeneous tumor harbor at least one mutation in at least one of the plurality of amplicons.

In some embodiments of the method, the P13K/AKT/mTor pathway inhibitor is selected from the group consisting of BKM120, BEZ235, Pictilisib (GDC-0941), LY294002, CAL-101 (Idelalisib), GNE-317, PI-3065, HS-173, PI-103, NU7441, GSK2636771, VS-5584, CZC24832, Duvelisib, TG100-115, A66, YM201636, CAY10505, GSK1059615, PF-04691502, PIK-75, PIK-93, AS-605240, BGT226, AZD6482, Voxtalisib, Alpelisib, CUDC-907, IC-87114, Omipalisib, TG100713, Gedatolisib, CH5132799, PKI-402, BAY 80-6946, TGX-221, XL147, PIK-90, PIK-293, PIK-294, 3-Methyladenine, Quercetin, Wortmannin, ZSTK474, AS-252424, AS-604850, everolimus, and Apitolisib.

In one particular embodiment, the subject is diagnosed as having HER-2 negative breast cancer. In some embodiments of the method, the at least one additional agent is selected from the group consisting of Notch pathway inhibitors, BRAF inhibitors, SMO antagonists, MET inhibitors, and ERBB2 antagonists. In certain embodiments, the Notch pathway inhibitors are selected from the group consisting of FLI-06, LY411575, Dibenzazepine, RO4929097, Compound E, Z-Leu-Leu-Nle-CHO, SAHM1, TR4 and Semagacestat. In some embodiments, the SMO antagonists are selected from the group consisting of Purmorphamine, Taladegib (LY2940680), Cyclopamine, Vismodegib (GDC-0449), LDE225, Glasdegib (PF-04449913), PF-5274857, TAK-441, SANT-1, BMS-833923, GANT61 and IPI-926.

In some embodiments, the ERBB2 antagonists are selected from the group consisting of Lapatinib, Canertinib, CP-724,714, AZD8931, AEE788, Tyrphostin AG 879, Mubritinib, and Pertuzumab. In certain embodiments, the BRAF inhibitors are selected from the group consisting of GDC-0879, SB590885, Encorafenib, RAF265, TAK-632, PLX4720, CEP-32496, AZ628, Sorafenib Tosylate, Sorafenib, Vemurafenib (Zelboraf) and Dabrafenib (GSK2118436).

In some embodiments of the method, the subject is diagnosed as having colorectal cancer. In a further embodiment, the at least one additional agent is selected from the group consisting of Notch pathway inhibitors, FGFR3 antagonists, and RAF/MEK/ERK inhibitors. In certain embodiments, the RAF/MEK/ERK inhibitors are selected from the group consisting of Vemurafenib (Zelboraf) and Dabrafenib (GSK2118436), Encorafenib, TAK-632, PLX4720, MLN2480, Cobimetinib (GDC-0973), MEK 162, RO5126766, GDC-0623, VTX11c, Selumctinib (AZD6244), PD0325901, Tramctinib (GSK1120212), U0126-EtOH, PD184352 (CI-1040), Refamctinib, PD98059, BIX02189, Binimctinib, Pimasertib (AS-703026), SL327, BIX02188, AZD8330, TAK-733, PD318088, SCH772984, and FR 180204.

In some embodiments, the Notch pathway inhibitors are selected from the group consisting of FLI-06, LY411575, Dibenzazepine, RO4929097, Compound E, Z-Leu-Leu-Nlc-CHO, SAHM1, TR4 and Semagacestat. In certain embodiments, the FGFR3 antagonists are selected from the group consisting of BGJ398 (NVP-BGJ398), AZD4547, LY2874455, Dovitinib Dilactic acid, Dovitinib, Dovitinib Lactate, CH5183284, and Nintedanib.

In one particular embodiment, a mutation in at least one of the amplicons corresponding to BRAF, MAP2K1 and NRAS and a mutation in at least one of the amplicons corresponding to FGFR3 and SMO are detected. In a further embodiment, the subject is diagnosed as having melanoma. In some embodiments, the at least one additional agent is RAF/MEK/ERK inhibitors, FGFR3 antagonists, SMO antagonists or a combination thereof.

In one aspect, the present disclosure provides a method for predicting the likelihood of lack of responsiveness to treatment with an anti-HER-2 therapy in a HER-2 positive subject diagnosed as having breast cancer comprising: (a) extracting genomic DNA from a formalin fixed paraffin-embedded specimen obtained from the HER-2 positive subject; (b) generating a library comprising amplicons corresponding to each of a plurality of cancer-related genes, said plurality of cancer-related genes comprising AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN, wherein (i) generating said library proceeds independently of using a bait set comprising nucleic acid sequences that are complementary to at least one of the plurality of amplicons, and (ii) the quality of the genomic DNA extracted from the formalin fixed paraffin-embedded specimen is not assessed using quantitative PCR prior to generating the library; (c) detecting at least one mutation in at least one of the plurality of amplicons; and (d) identifying the HER-2 positive subject as having a likelihood of lack of responsiveness to treatment with an anti-HER-2 therapy, when a mutation in at least one of the amplicons corresponding to PIK3CA, PIK3R1 and PTEN is detected. In some embodiments of the method, the anti-HER-2 therapy is trastuzumab or lapatinib.

In some embodiments of the method, the amplicons corresponding to PIK3CA are generated by a pair of primers selected from the group consisting of 5′ CCTAGTAGAATGTTTACTACCAA 3′ (SEQ ID NO.: 1) and 5′ CTGCTTCTTGAGTAACACTT 3′ (SEQ ID NO.: 2); 5′ CATGTTCATGCTGTGTATGT 3′ (SEQ ID NO.: 3) and 5′ GCTTCTTTACAAACGTTCAGAA 3′ (SEQ ID NO.: 4); 5′ TCTATGTTCGAACAGGTATCT 3′ (SEQ ID NO.: 5) and 5′ ACTGCTAAACACTAATATAACCTTTG 3′ (SEQ ID NO.: 6); 5′ TTGAAATGTGTTTTATAATTTAGACTAGT 3′ (SEQ ID NO.: 7) and 5′ CCATGAGGTACTGGCC 3′ (SEQ ID NO.: 8); 5′ TTGGTGTTACTGGATCAAATC 3′ (SEQ ID NO.: 9) and 5′ TGCTGAACCAGTCAAACT 3′ (SEQ ID NO.: 10); 5′ TATTATTTTATTTTACAGAGTAACAGACTAG 3′ (SEQ ID NO.: 11) and 5′ TTTAGCACTTACCTGTGACT 3′ (SEQ ID NO.: 12); 5′ TGGAATGCCAGAACTACA 3′ (SEQ ID NO.: 13) and 5′ GTGGAAGATCCAATCCATTTT 3′ (SEQ ID NO.: 14); 5′ GGAATGAATGGCTGAATTATG 3′ (SEQ ID NO.: 15) and 5′ GCGGTATAATCAGGAGTTTT 3′ (SEQ ID NO.: 16); 5′ AGTTGGCCTGAATCACTATA 3′ (SEQ ID NO.: 17) and 5′ GATGTTACTATTGTGACGATCTC 3′ (SEQ ID NO.: 18); 5′ GTAAGTGTTACTCAAGAAGC 3′ (SEQ ID NO.: 19) and 5′ ATAGGATATTGTATCATACCAATTTCT 3′ (SEQ ID NO.: 20); 5′ TCCACAGCTACACCATATAT 3′ (SEQ ID NO.: 21) and 5′ AGCATCAGCATTTGACTTTA 3′ (SEQ ID NO.: 22); 5′ TACACAGACACTCTAGTATCTG 3′ (SEQ ID NO.: 23) and 5′ GAAGGTTTGACTGCCATAAA 3′ (SEQ ID NO.: 24); 5′ ATGACAAAGAACAGCTCAAA 3′ (SEQ ID NO.: 25) and 5′ GAGATCAGCCAAATTCAGTT 3′ (SEQ ID NO.: 26); 5′ GATGTGTTACAAGGCTTATCTA 3′ (SEQ ID NO.: 27) and 5′ GCCTCTTGCTCAGTTTTATC 3′ (SEQ ID NO.: 28); 5′ GAGGCTTTGGAGTATTTCA 3′ (SEQ ID NO.: 29) and 5′ CTGCTGAGAGTTATTAACAGT 3′ (SEQ ID NO.: 30); and 5′ GCTTTTGGAGTCCTATTGT 3′ (SEQ ID NO.: 31) and 5′ CACAAACTAGAGTCACACAC 3′ (SEQ ID NO.: 32).

In certain embodiments, the HER-2 positive subject is treated with trastuzumab emtansine, when a mutation in at least one of the amplicons corresponding to PIK3CA, PIK3R1 and PTEN is detected.

In some embodiments, the HER-2 positive status of the subject is assayed by immunohistochemistry (IHC) or fluorescent in situ hybridization (FISH).

In another aspect, the present disclosure provides a method for selecting a subject for treatment with a EGFR tyrosine kinase inhibitor and at least one additional agent comprising: (a) extracting genomic DNA from a formalin fixed paraffin-embedded specimen obtained from the subject; (b) generating a library comprising amplicons corresponding to each of a plurality of cancer-related genes, said plurality of cancer-related genes comprising AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN, wherein (i) generating said library proceeds independently of using a bait set comprising nucleic acid sequences that are complementary to at least one of the plurality of amplicons, and (ii) the quality of the genomic DNA extracted from the formalin fixed paraffin-embedded specimen is not assessed using quantitative PCR prior to generating the library; (c) detecting at least one mutation in at least one of the plurality of amplicons; and (d) selecting the subject for treatment with a EGFR tyrosine kinase inhibitor and at least one additional agent, if a mutation in at least one of the amplicons corresponding to EGFR, and a mutation in at least one of the amplicons corresponding to KRAS, PIK3R1 and BRAF are detected.

In some embodiments of the method, the formalin fixed paraffin-embedded specimen is a heterogeneous tumor. In some embodiments, 5%-10% of the cells of the heterogeneous tumor harbor at least one mutation in at least one of the plurality of amplicons. In other embodiments, at least 10% of the cells of the heterogeneous tumor harbor at least one mutation in at least one of the plurality of amplicons.

In certain embodiments, the EGFR tyrosine kinase inhibitor is gefitinib or erlotinib.

In one aspect, the present disclosure provides a method for predicting the likelihood of responsiveness to treatment with vemurafenib in a subject diagnosed as having melanoma comprising: (a) extracting genomic DNA from a formalin fixed paraffin-embedded specimen obtained from the subject; (b) generating a library comprising amplicons corresponding to each of a plurality of cancer-related genes, said plurality of cancer-related genes comprising AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN, wherein (i) generating said library proceeds independently of using a bait set comprising nucleic acid sequences that are complementary to at least one of the plurality of amplicons, and (ii) the quality of the genomic DNA extracted from the formalin fixed paraffin-embedded specimen is not assessed using quantitative PCR prior to generating the library; (c) detecting at least one mutation in at least one of the plurality of amplicons; and (d) identifying the subject as having at least one of a high likelihood of responsiveness to treatment with vemurafenib when a mutation in at least one of the amplicons corresponding to BRAF is detected, and a low likelihood of responsiveness to treatment with vemurafenib when a mutation in at least one of the amplicons corresponding to NRAS is detected.

In one aspect, the present disclosure provides a method for predicting the likelihood of responsiveness to treatment with anti-EGFR therapy in a subject diagnosed as having colorectal cancer comprising: (a) extracting genomic DNA from a formalin fixed paraffin-embedded specimen obtained from the subject; (b) generating a library comprising amplicons corresponding to each of a plurality of cancer-related genes, said plurality of cancer-related genes comprising AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN, wherein (i) generating said library proceeds independently of using a bait set comprising nucleic acid sequences that are complementary to at least one of the plurality of amplicons, and (ii) the quality of the genomic DNA extracted from the formalin fixed paraffin-embedded specimen is not assessed using quantitative PCR prior to generating the library; (c) detecting at least one mutation in at least one of the plurality of amplicons; and (d) identifying the subject as having a low likelihood of responsiveness to treatment with anti-EGFR therapy when a mutation in at least one of the amplicons corresponding to KRAS, BRAF, NRAS, PIK3CA, and PTEN is detected.

### BRIEF DESCRIPTION OF THE DRAWINGS

FIG. 1 shows the clinical characteristics of the 121 patients that provided the tumor samples that were analyzed in the present study.

FIG. 2 shows the results of the inter-assay precision experiments with FFPE specimens harboring variants BRAF G466Y, TP53 R175H, DDR2 L34P, EGFR E865G, EGFR E866V, TP53 R248W, Notch Q24064, and TP53 A159_M160insRA.

FIG. 3 shows the results of the intra-assay precision experiments with cell lines harboring variants EGFR ΔE746_A750, EGFR L858R, and AKT1 E17K.

FIG. 4 is a Venn diagram that summarizes the mutated genes detected by the solid tumor screening assay of the present technology according to tumor type: lung cancer, colorectal cancer, melanoma, and breast cancer. Genes that were mutated in several tumor types are depicted in their respective regions of overlap where possible.

FIG. 5 shows the percentage of FFPE samples by tumor type that harbor multiple co-occurring mutations (as detected by the solid tumor screening panel of the present technology). No tumor sample harbored ≥5 co-occurring mutations.

FIGS. 6A-6E show the mutations detected by the solid tumor screening panel of the present technology for each individual specimen (columns) for (FIG. 6A) all tumor types, (FIG. 6B) melanoma, (FIG. 6C) colorectal cancer, (FIG. 6D) lung cancer, and (FIG. 6E) breast cancer. The total number of specimens tested for a given tumor type is also shown. The number of specimens harboring mutations in the gene represented by a given row is provided for each panel (labeled on the right axes for all panels). Specimens harboring 2 mutations within the same gene are also shown. Clinical laboratory results for each specimen are indicated by the figure legend.

FIG. 7 shows the molecular profile of 28 breast cancer tumor samples that were initially screened for ER, PR and HER-2 expression.

### DETAILED DESCRIPTION

The present disclosure provides methods for determining whether a patient diagnosed with breast cancer, colorectal cancer, melanoma or lung cancer will benefit from or is predicted to be responsive to treatment with an individual therapeutic agent or a specific combination of therapeutic agents. These methods are based on screening a patient's solid tumors and detecting alterations in target nucleic acid sequences corresponding to a specific set of cancer-related genes using a highly sensitive Next-Generation Sequencing (NGS) PCR-based assay. Kits for use in practicing the methods are also provided.

Molecular profiling of tumors is becoming increasingly important in the management of advanced cancer. NGS is widely used in cancer research and has become an attractive diagnostic technology in clinical laboratories because of its ability to detect multiple variants in a single assay.

FFPE specimens are integral to the diagnosis of virtually every suspected cancer case, and the estimated millions of archived samples can provide a wealth of molecular information about disease progression and treatment. While FFPE techniques are the standard for protecting tissues for downstream molecular analysis and case of archiving, storage of tissues in formaldehyde solution results in extensive crosslinking of proteins to other proteins and to nucleic acids and in nucleic acid fragmentation. FFPE techniques can result in the partial denaturation of the DNA and may cause damage to the DNA base pairs themselves, thereby compromising the accuracy of NGS assays. Moreover, the amount of tumor tissue available for biopsy is often limited. These challenges are further exacerbated by the extent of molecular heterogeneity observed in tumor samples, which makes the detection of actionable genetic alterations in FFPE tissues extremely difficult.

Thus, the limited amount of high-quality DNA obtained from FFPE tumor samples disincentivizes the use of large-scale solid tumor screening panels which require large quantities of input DNA (e.g., 50 ng for FoundationOne® panel that interrogates the entire coding sequence of over 300 cancer-related genes and 28 gene rearrangements, Foundation Medicine®). Moreover, analysis of cancer-related genes that are commonly associated with hematologic variants (e.g., ABL1) are less likely to provide guidance on targeted treatment strategies for genetically heterogeneous solid tumors. Accordingly, there is a need for more focused solid tumor screening panels that provide accurate and clinically relevant information on heterogeneous solid tumors but are economical in terms of their use of input DNA from FFPE samples.

One objective of the present technology was to develop a highly sensitive solid tumor profiling panel that can simultaneously detect a broad range of mutations in specifically targeted exons or gene regions of a preselected set of cancer-related genes that are currently, or are likely to become, therapeutically actionable in solid tumors. In some embodiments, the solid tumors manifest in patients diagnosed as having melanoma, lung cancer, colorectal cancer, breast cancer, thyroid cancer, gastrointestinal stromal tumors, etc.

The present disclosure provides methods for detecting actionable genetic mutations in a specific set of cancer-related genes in solid tumors (derived from FFPE tissues) that are missed by traditional Sanger sequencing methods. In one aspect, the methods of the present technology are useful in detecting genetic alterations in a specific set of cancer-related genes in highly heterogeneous tumors samples. Further, the methods disclosed herein are less labor intensive, require less DNA input from FFPE specimens, and provide additional insights into how different signaling pathways are impacted within a particular tumor sample (e.g., a heterogeneous tumor) compared to other existing NGS PCR-based tumor screening assays. In particular, the methods of the present technology screen for mutations in specific target regions within 34 preselected cancer-related genes, which in turn provide a general overview of the molecular profile of a FFPE tumor sample (e.g., a heterogeneous tumor), without prior assessment of the quality of the genomic DNA extracted from the FFPE tumor sample.

The methods disclosed herein are useful in (a) predicting the responsiveness of a subject diagnosed with breast cancer, colorectal cancer, melanoma or lung cancer to a particular therapeutic agent, and (b) selecting optimal treatment strategies for the subject in light of the nature of the individual subject's tumor. Accordingly, DNA degradation/partial DNA denaturation during the FFPE process and tumor heterogeneity do not appear to influence the sensitivity of the solid tumor screening assay of the present technology.

### Definitions

As used herein, the term “about” in reference to a number is generally taken to include numbers that fall within a range of 1%-5% in either direction (greater than or less than) of the number unless otherwise stated or otherwise evident from the context.

As used herein, the terms “amplify” or “amplification” with respect to nucleic acid sequences, refer to methods that increase the representation of a population of nucleic acid sequences in a sample. Nucleic acid amplification methods, such as PCR, isothermal methods, rolling circle methods, etc., are well known to the skilled artisan. Copies of a particular nucleic acid sequence generated in vitro in an amplification reaction are called “amplicons” or “amplification products”.

As used herein, the term “actionable genetic alterations” refers to mutations that are associated with (1) treatment with an FDA approved drug, (2) a guideline supported drug treatment, (3) a guideline indication of sensitivity or resistance to a particular treatment, (4) ongoing clinical trials, (5) clinical data supporting an indication of resistance or sensitivity to drug treatment, (6) pre-clinical data showing strong evidence of resistance or sensitivity to a targeted treatment, or (6) a prognostic implication that may guide a physician's treatment decisions.

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing.

As used herein, an “alteration” of a gene or gene product (e.g., a marker gene or gene product) refers to the presence of a mutation or mutations within the gene or gene product, e.g., a mutation, which affects the quantity or activity of the gene or gene product, as compared to the normal or wild-type gene. The genetic alteration can result in changes in the quantity, structure, and/or activity of the gene or gene product in a cancer tissue or cancer cell, as compared to its quantity, structure, and/or activity, in a normal or healthy tissue or cell (e.g., a control). For example, an alteration which is associated with cancer, or predictive of responsiveness to anti-cancer therapeutics, can have an altered nucleotide sequence (e.g., a mutation), amino acid sequence, chromosomal translocation, intra-chromosomal inversion, copy number, expression level, protein level, protein activity, in a cancer tissue or cancer cell, as compared to a normal, healthy tissue or cell. Exemplary mutations include, but are not limited to, point mutations (e.g., silent, missense, or nonsense), deletions, insertions, inversions, linking mutations, duplications, translocations, inter- and intra-chromosomal rearrangements. Mutations can be present in the coding or non-coding region of the gene. In certain embodiments, the alterations are associated with a phenotype, e.g., a cancerous phenotype (e.g., one or more of cancer risk, cancer progression, cancer treatment or resistance to cancer treatment). In one embodiment, the alteration is associated with one or more of: a genetic risk factor for cancer, a positive treatment response predictor, a negative treatment response predictor, a positive prognostic factor, a negative prognostic factor, or a diagnostic factor.

“Bait”, as used herein, is a type of hybrid capture reagent that retrieves target nucleic acid sequences for sequencing. A bait can be a nucleic acid molecule, e.g., a DNA or RNA molecule, which can hybridize to (e.g., be complementary to), and thereby allow capture of a target nucleic acid. In one embodiment, a bait is an RNA molecule (e.g., a naturally-occurring or modified RNA molecule); a DNA molecule (e.g., a naturally-occurring or modified DNA molecule), or a combination thereof. In other embodiments, a bait includes a binding entity, e.g., an affinity tag, that allows capture and separation, e.g., by binding to a binding entity, of a hybrid formed by a bait and a nucleic acid hybridized to the bait. In one embodiment, a bait is suitable for solution phase hybridization.

As used herein, “bait set” refers to one or a plurality of bait molecules.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, a “control” is an alternative sample used in an experiment for comparison purpose. A control can be “positive” or “negative.” A “control nucleic acid sample” or “reference nucleic acid sample” as used herein, refers to nucleic acid molecules from a control or reference sample. In certain embodiments, the reference or control nucleic acid sample is a wild type or a non-mutated DNA or RNA sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-cancerous sample from the same or a different subject.

“Detecting” as used herein refers to determining the presence of a mutation or alteration in a nucleic acid of interest in a sample. Detection does not require the method to provide 100% sensitivity.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

The term “heterogeneous tumor” as used herein refers to a tumor that comprises subpopulations of cells with distinct molecular variations (e.g., subclones with varying genotypes). In some embodiments, the heterogeneous tumor cells may also exhibit distinct phenotypic profiles including differences in cellular morphology, gene expression, metabolism, motility, proliferation, and metastatic potential. In some embodiments, the degree of heterogeneity within a tumor may depend on the tissue in which the tumor manifests.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tm) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N. Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the terms “individual”, “patient”, or “subject” are used interchangeably and refer to an individual organism, a vertebrate, a mammal, or a human. In a preferred embodiment, the individual, patient or subject is a human.

As used herein, the term “library” refers to a collection of nucleic acid sequences, e.g., a collection of nucleic acids derived from whole genomic, subgenomic fragments, cDNA, cDNA fragments, RNA, RNA fragments, or a combination thereof. In one embodiment, a portion or all of the library nucleic acid sequences comprises an adapter sequence. The adapter sequence can be located at one or both ends. The adapter sequence can be useful, e.g., for a sequencing method (e.g., an NGS method), for amplification, for reverse transcription, or for cloning into a vector.

The library can comprise a collection of nucleic acid sequences, e.g., a target nucleic acid sequence (e.g., a tumor nucleic acid sequence), a reference nucleic acid sequence, or a combination thereof). In some embodiments, the nucleic acid sequences of the library can be derived from a single subject. In other embodiments, a library can comprise nucleic acid sequences from more than one subject (e.g., 2, 3, 4, 5, 6, 7, 8, 9, 10, 20, 30 or more subjects). In some embodiments, two or more libraries from different subjects can be combined to form a library having nucleic acid sequences from more than one subject. In one embodiment, the subject is human having, or at risk of having, a cancer or tumor.

A “library nucleic acid sequence” refers to a nucleic acid molecule, e.g., a DNA, RNA, or a combination thereof, that is a member of a library. Typically, a library nucleic acid sequence is a DNA molecule, e.g., genomic DNA or cDNA. In some embodiments, a library nucleic acid sequence is fragmented, e.g., sheared or enzymatically prepared, genomic DNA. In certain embodiments, the library nucleic acid sequences comprise sequence from a subject and sequence not derived from the subject, e.g., adapter sequence, a primer sequence, or other sequences that allow for identification, e.g., “barcode” sequences.

The term “multiplex PCR” as used herein refers to amplification of two or more PCR products or amplicons which are each primed using a distinct primer pair.

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 10³, 10⁴, 10⁵ or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11:31-46 (2010).

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of dsDNA. A “reverse primer” anneals to the sense-strand of dsDNA.

As used herein, “primer pair” refers to a forward and reverse primer pair (i.e., a left and right primer pair) that can be used together to amplify a given region of a nucleic acid of interest.

As used herein, a “sample” refers to a substance that is being assayed for the presence of a mutation in a nucleic acid of interest. Processing methods to release or otherwise make available a nucleic acid for detection are well known in the art and may include steps of nucleic acid manipulation. A biological sample may be a body fluid or a tissue sample. In some cases, a biological sample may consist of or comprise blood, plasma, sera, urine, feces, epidermal sample, vaginal sample, skin sample, check swab, sperm, amniotic fluid, cultured cells, bone marrow sample, tumor biopsies, aspirate and/or chorionic villi, cultured cells, and the like. Fresh, fixed or frozen tissues may also be used. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. Whole blood samples of about 0.5 to 5 ml collected with EDTA, ACD or heparin as anti-coagulant are suitable.

The term “sensitivity,” as used herein in reference to the methods of the present technology, is a measure of the ability of a method to detect a preselected sequence variant in a heterogeneous population of sequences. A method has a sensitivity of S % for variants of F % if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%).

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity.

As used herein, a “solid tumor” is an abnormal mass of tissue that usually does not contain cysts or liquid areas. Solid tumors may be benign, or malignant (cancer). Examples of solid tumors are sarcomas, carcinomas, and lymphomas. A solid tumor is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

Specific criteria for the staging of cancer are dependent on the specific cancer type based on tumor size, histological characteristics, tumor markers, and other criteria known by those of skill in the art. Generally, cancer stages can be described as follows:

Stage 0. Carcinoma in situ

Stage I, Stage II, and Stage III. Higher numbers indicate more extensive disease: Larger tumor size and/or spread of the cancer beyond the organ in which it first developed to nearby lymph nodes and/or tissues or organs adjacent to the location of the primary tumor

Stage IV. The cancer has spread to distant tissues or organs

“Specificity,” as used herein, is a measure of the ability of a method to distinguish a truly occurring preselected sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N_{Total} sequences, in which X_{True} sequences are truly variant and X_{Not} true are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include 90, 95, 98, and 99%.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “target sequence” and “target nucleic acid sequence” refer to a specific nucleic acid sequence to be detected and/or quantified in the sample to be analyzed.

As used herein, the terms “treat,” “treating” or “treatment” refer, to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) state of disease, amelioration or palliation of the disease state, diminishing rate of or time to progression, and remission (whether partial or total).

### Impact of Tumor Profiling on Targeted Drug Therapies

Cancer has become the focus for drug development over the past decade, with the number of new cancer drugs in development tripling from 2001 to 2010. Siegel et al., CA Cancer J Clin. 64:9-29 (2014). The modest survival rates and high degree of adverse effects from standard chemotherapy and radiation treatment have led to focus new drug developments on treatments that target specific signaling molecules or whole regulatory pathways. The efficacy of many targeted drugs can be influenced by molecular biomarkers, which are now commonly used as an aid in selecting patients for treatment.

Furthermore, mutational heterogeneity of solid tumors has broadened the scope of cell signaling pathways that are targeted by new therapeutics. Hanahan & Weinberg, Cell 144:646-74 (2011); Fisher et al., British J. Cancer 108:479-485 (2013); Burrella & Swantona, Molecular Oncology 8:1095-1111 (2014). Identifying pathway alterations could steer a clinician towards or away from drugs targeting the affected pathways. Furthermore, without wishing to be bound by theory, an overlap in affected regulatory pathways among cancers may indicate that approved biomarkers and drug treatments for one tumor type may have potential clinical applications in other tumor types. One example of successfully applying a single targeted treatment across multiple tumor types and biomarker indications is the use of the tyrosine kinase inhibitor imatinib (imatinib mesylate). Imatinib was originally developed to target c-abl in chronic myeloid leukemias (CMLs) harboring the Philadelphia chromosome (BCR/ABL1), but its indications have since been expanded to include the treatment of certain GIST tumors harboring KIT or PDGFRA mutations as well as advanced or metastatic melanomas harboring KIT mutations. Peng et al., Clinical Pharmacokinetics 44:879-894 (2005); Guo et al., JCO 29:2904-2909 (2011). Accordingly, there might be a potential benefit to prospectively profiling tumors for targets that might not, at the moment, be clinically actionable for a given tumor type.

Mutations in “downstream” signaling proteins can also cause drug resistance, lending additional utility to mutation profiling. Kelloff & Sigman, Nat Rev Drug Discov. 11:201-214 (2012). Profiling tumors that exhibit acquired resistance has led to the identification of mechanisms of resistance. For example, a melanoma patient with acquired resistance to the BRAF inhibitor vemurafenib was found to harbor a point mutation in MEK1 C121S. Wagle et al., Cancer Discovery 4:61-68 (2014); Narita et al., Mol Cancer Ther. 13:823-832 (2014). MEK1 encodes a kinase downstream of BRAF, and the C121S mutation was believed to account for vemurafenib resistance in the aforementioned case. Although no alternative drug was available at the time, a novel MEK1 inhibitor in preclinical development was recently reported to be active against vemurafenib-resistant melanoma harboring the MEK1 C121S mutation. Wagle et al., J Clin Oncol. 29:3085-3096 (2011). Cases such as this highlight the potential utility of identifying mutations in a broad range of cancer-associated genes in patients exhibiting acquired resistance, even those not yet linked to the therapeutic response.

Treatment-naïve patients also frequently have concurrent mutations in multiple genes that may have implications for prognosis or treatment decisions. For example, concurrent KRAS and PIK3CA mutations are commonly found in colorectal and lung cancers, and the combinatorial status may have prognostic or treatment predictive value. Roock et al., Lancet Oncology 12:594-603 (2011); Chaft et al., Mol Cancer Ther. 11; 485 (2012); Jänne P A et al., Lancet Oncol. 14:38-47 (2013). Concurrent PIK3CA mutations in EGFR-positive lung cancers were described to be associated with resistance to EGFR tyrosine kinase inhibitors (TKIs). Ludovini et al., J. Thoracic Oncology 6:707-715 (2011). Moreover, a recent study demonstrated that 20% of ALK rearrangement-positive lung cancers harbored co-occurring mutations in EGFR or MET, which may be an important factor in the decision to treat with crizotinib. Boland et al., J. Thoracic Oncology 8:574-581 (2013). Thus, prospectively profiling tumors would aid in the selection of optimal therapeutic regimens, thereby improving the likelihood of a positive patient outcome.

### NGS Platforms

In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing. Examples of Next Generation Sequencing techniques include, but are not limited to pyrosequencing, Reversible dye-terminator sequencing, SOLID sequencing, Ion semiconductor sequencing, Helioscope single molecule sequencing etc.

The Ion Torrent™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454TM GS FLX™ sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 454TM system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added polyA tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLID™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

### Solid Tumor Screening Methods of the Present Technology

Disclosed herein are methods and assays that are based, at least in part, on a pre-selected set of genes that are associated with a cancerous phenotype (e.g., one or more of cancer risk, cancer progression, cancer treatment response or resistance to cancer treatment). Such pre-selected genes enable the application of sequencing methods, particularly methods that rely on massively parallel sequencing of a large number of diverse genes, e.g., from tumor or control samples.

In one embodiment, the methods featured in the present technology are used in a multiplex, multi-gene assay format, e.g., assays that incorporate multiple signals from a large number of diverse genetic alterations in a large number of genes.

The methods of the present technology are based on the principle that assaying cell populations within a tumor sample, e.g., a heterogeneous solid tumor, for the presence of one or more alterations in a preselected set of cancer-related genes is useful in determining whether a patient will benefit from or will respond to treatment with an individual therapeutic agent or a specific combination of therapeutic agents. In some embodiments of the method, the preselected set of cancer-related genes corresponds to AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN. In some embodiments of the method, the presence of one or more alterations in the preselected set of cancer-related genes is detected by assaying a plurality of amplicons corresponding to the preselected set of cancer-related genes.

The advantages of the methods of the present technology over other comparable PCR-based NGS screening panels for solid tumors are two-fold. First, no preliminary assessment of the quality of the genomic DNA extracted from FFPE tissues is required in order to generate highly uniform amplicon-based libraries. Second, the minimal DNA input required for the screening assays disclosed herein is about three times lower than other comparable solid tumor screening panels. For example, the minimal input DNA for the methods disclosed herein is 10 ng for generating 231 amplicons corresponding to 34 genes, whereas the TruSight™ Tumor protocol requires at least 30 ng for generating 174 amplicons corresponding to 26 genes.

The present disclosure provides methods for detecting at least one mutation in a plurality of cancer-related genes in a subject comprising (a) extracting genomic DNA from a FFPE tumor sample obtained from the subject; (b) generating a library comprising amplicons corresponding to each of the plurality of cancer-related genes, said plurality of cancer-related genes comprising AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN, wherein (i) generating said library occurs without the use of a bait set comprising nucleic acid sequences that are complementary to at least one of the plurality of amplicons; and (ii) the quality of the genomic DNA extracted from the FFPE tumor sample is not assessed using quantitative PCR prior to generating the library; (c) ligating an adapter sequence to the ends of the plurality of amplicons; and (d) detecting at least one mutation in at least one of the plurality of amplicons using high throughput massive parallel sequencing.

In some embodiments of the method, the subject has been diagnosed as having, is suspected of having, or is at risk of having breast cancer, melanoma, colorectal cancer or lung cancer.

In some embodiments of the method, the at least one mutation detected is a mutation in EGFR, KRAS, BRAF, NRAS, ERBB2 or PIK3CA. In one embodiment, the at least one mutation detected is selected from the group consisting of BRAF V600E, BRAF V600K, BRAF K483Q, BRAF G466V, BRAF G464V, BRAF E501V, BRAF E501K, EGFR ΔE746_A750, EGFR R680Q, EGFR G598E, KRAS A146T, KRAS R68M, KRAS L19F, KRAS G12V, KRAS G12D, KRAS G12C, KRAS G13D, KRAS G13C, KRAS G12A, KRAS G12S, KRAS Q22K, NRAS Q61K, NRAS Q61R, NRAS G12R, NRAS G12D, PIK3CA C420R, PIK3CA G106R, PIK3CA R38H, PIK3CA E453K, PIK3CA H1044R, PIK3CA N1044K, PIK3CA E545K, PIK3CA Q546H, PIK3CA H1047R, PIK3CA H1043L, PIK3CA M1043V, PIK3CA E542K, PIK3CA E542Q, PIK3CA T1053A, PIK3CA 1121V, PIK3CA H1047L, ERBB2 L755S, ERBB2 S310Y, ERBB2 D769Y, ERBB2 S255R, DDR2 H92Y, DDR2 R31L, DDR2 L34P, DDR2 P381R and DDR2 K392N.

In some embodiments of the method, the library comprising amplicons corresponding to each of the plurality of cancer-related genes disclosed herein is generated using no more than 10 ng of extracted genomic DNA from the FFPE tumor sample. In some embodiments of the method, the library comprising amplicons corresponding to each of the plurality of cancer-related genes disclosed herein is generated using 11-25 ng of extracted genomic DNA from the FFPE tumor sample. In some embodiments of the method, the library comprising amplicons corresponding to each of the plurality of cancer-related genes disclosed herein is generated using at least 25 ng of extracted genomic DNA from the FFPE tumor sample.

In some embodiments of the method, the plurality of amplicons are generated by at least two, at least three, at least four, at least five, at least ten, at least twenty, at least thirty, at least forty, at least fifty, or at least one hundred or more pairs of primers disclosed in Table 1.

TABLE 1
Primer Pair Mix Pool 1

| | SEQ ID | | SEQ ID |
|---|---|---|---|
| Forward Primer Sequence | NO: | Reverse Primer Sequence | NO: |
| CTGAGGTGGAAGAGACAG | 83 | GCATTTCCTTTCTTCCCAG | 179 |
| ACTGGTTCTCACTCACC | 84 | TTGGTTACATCCCTCTCTG | 180 |
| GCCAGCAAAGCAGTAG | 85 | TGTTGCAGCTGACCAC | 181 |
| TGGAAAAATAGCCTCAATTCT | 86 | TGTTTTCCTTTACTTACTACACC | 182 |
| CACCACATTACATACTTACCAT | 87 | TTTGGCTTGACTTGACTTTT | 183 |
| CTCTGGAAAAGAGTAATTCACA | 88 | TGAAATGACACTTGGAGTAAC | 184 |
| CGTATTTATAGCTGATTTGATGG | 89 | GTATCCACATCCTCTTCCT | 185 |
| GGACTTCACCTGACAGAT | 90 | TGGTCAGTACAAGCACATAC | 186 |
| CTTTGGTTTCTCTTGGTCTAG | 91 | ATGGGCTTTCTTGATGTAAC | 187 |
| GAGTTTCTGCAGATTGACTT | 92 | GGTTCCGCCAAGAGAT | 188 |
| GTGTTTCCTTTGCAGATG | 93 | GGCCAATTTCCCATTCTAATA | 189 |
| TCACATGCCTCTTTCTCTA | 94 | TGACAAACCGAGCACT | 190 |
| CCTGGCTCTGACTCAC | 95 | CTTCATCCCCCAGTAAGTC | 191 |
| TTTCCCTGCCAAGTGAT | 96 | TTCTGGGAGTTTTCGTATCA | 192 |
| TGCAGCGGTGTTGT | 97 | TCCACAGCCACATCTTT | 193 |
| TCTCGGCTCAAGGAC | 98 | CTGACAGTGCGTACATC | 194 |
| TTCCTCAGTTACACCAATCT | 99 | TCATTCCAAAGTCAGCTATCT | 195 |
| GCCTTTGGGGTTACTTT | 100 | AGGTCCACATCCATTCATC | 196 |
| GCTGCTGGAGAAGAGATA | 101 | CATAGGCATGGGTGAGT | 197 |
| AGGTGGCTGGTTATGTC | 102 | CCTTCAGTCCGGTTTTATTTG | 198 |
| GGAGATAAGTGATGGAGATGT | 103 | CAGAAAGCGGTGACTTACT | 199 |
| GGATCGGCCTCTTCAT | 104 | GCTAATGGCCCGTTCTC | 200 |
| CCCAACCAAGCTCTCTT | 105 | CCCACCAGACCATGAGA | 201 |
| ACTGACGTGCCTCTC | 106 | AGCCAATATTGTCTTTGTGT | 202 |
| GCAGGGTCTTCTCTGTT | 107 | CTCCTTCTGCATGGTATTCT | 203 |
| CCAACAGAGGGAAACTAATAG | 108 | CATTAGCATCAGGATTATGACT | 204 |
| CTGATGGGGAGAATGTGA | 109 | CCAGAGCCCAGACCTG | 205 |
| CATTTGACCATGACCATGTA | 110 | GGAGTTACTATATGGGAACTGAT | 206 |
| CCCATGAATACCAGTGACTA | 111 | AACTCTGAGTCTTGTTTCTACA | 207 |
| CTGTTACTTACGTGGACATT | 112 | CCTGCATGAATTTCAATGAC | 208 |
| CTCATTCATCGCCACATAG | 113 | GTTATGCAGACACCATTCAT | 209 |
| GACTTACCTTGCAATGTTTG | 114 | AATTGCATTCACACGTTAAC | 210 |
| ACTGGCCTGTCTCAATAT | 115 | CCTTGACTAAATCTACCATGTTT | 211 |
| AGTGGAAGTATGCCCATATA | 116 | CCAGTGTCTGAGAACATTAGT | 212 |
| TACAGAAGAGGAGTGTCATAT | 117 | AGACAGCACAGAATTGATAC | 213 |
| TGCCCACTGTGTTACT | 118 | TCAGTGTATTCATCGAGATTTAG | 214 |
| AGTTGTGGGTACCTTTAGATT | 119 | TGTCTGCAAGGTTTACAGT | 215 |
| GGCGGTGGTGGT | 120 | GCTGTGCGTCACTGTA | 216 |
| GCCAGGCCTCAACG | 121 | CGCAGGCGGCAGA | 217 |
| GTGACCGAGGACAAC | 122 | GGAAGGCGGTGTTG | 218 |
| CTGACCGACGTTGAC | 123 | GCCTGCTGTGGCCC | 219 |
| CCTGGGATTGCAGATTG | 124 | AGGGCGACGAGAAAC | 220 |
| GTTTCATGGACTCAGTTACT | 125 | TCCTCTAGCTATCTTAATGACT | 221 |
| CCAGACCTTTGCTTTAGATT | 126 | GCTTACTGGAAGTTGACTTT | 222 |
| AAAGACTTGGTGTTGTTGAT | 127 | GGAAGCAGGTGGTCATT | 223 |
| GCCCATCATATTTCTTCAGA | 128 | TGGCTTCTCCTCTACAGA | 224 |
| CGTGCCACCCAGAATAT | 129 | GGATGCTGCAGAAGCTATAA | 225 |
| GGCCTACCTGGTCG | 130 | GTTCAAGCTGAAGAAGATGT | 226 |
| TTCCAGCACTCTGACATAT | 131 | GCTCAGTTCCTGGACAAA | 227 |
| TGCTTTTAGGGCCCAC | 132 | CCTTTGAATGCAGAAGATTCTT | 228 |
| CCAACCTAATAGTGTATTCACA | 133 | AGCAGAGAATGGGTACTC | 229 |
| ATTATTGACTCTGTTGTGCT | 134 | GTTGTCTTTGGCAAGGAT | 230 |
| CCCCAGTCCTCATGTAC | 135 | GCACTGTAATAATCCAGACTG | 231 |
| GCTGGAGGAGCTAGAG | 136 | GTGGGAGACCTTGAACA | 232 |
| CCTTCTCTTCCCCAATCTAC | 137 | GCCATGGAGTCGATGAG | 233 |
| AGGAGCCAGGCATTTT | 138 | GCCTGACAAATCCAGAGTA | 234 |
| CTTTCCCCACAATCATACTG | 139 | TGATGAACCGGTCCTTTAC | 235 |
| GCTGGTGTTGTCTCAATAT | 140 | CGACGACAATCTTAAACTGTA | 236 |
| TACGATGCAAGAGTACACA | 141 | ACAACCCACTGAGGTATATG | 237 |
| CCACCACTGGATTTCTCA | 142 | TGTATGGTAGGACCACCAG | 238 |
| CTTATTGGCTTTGGTCTTCA | 143 | AAACAGATTCCTCCTTGTCA | 239 |
| TCAGCATCATTGTAAATTATTCTATTT | 144 | GCACCTGTTTTGTTGTGTA | 240 |
| ACGAGCTGGACCACT | 145 | CCCACCCGTGACCG | 241 |
| TCGGCTCTCCACTCA | 146 | CAGGTGCAGCCACAAA | 242 |
| GGGCCACACTTACTCT | 147 | TCTACCTGGAGATTGACAAC | 243 |
| CCGGGTCTCACTCA | 148 | CAAGCACCCCATCAAG | 244 |
| CGTAGTAGGGGAAGATCATC | 149 | CCGGCACGCTGGT | 245 |
| GGACACTCGCAGTAGAA | 150 | CCACGGTGGCTACAA | 246 |
| ATAATGCTCCTAGTACCTGTAG | 151 | ACCTGTTTGTTGGACATACT | 247 |
| CTTGCACAAATGCTGAAAG | 152 | AAGACTCGGATGATGTACC | 248 |
| CATTTATAGAAACCGAGGTATGA | 153 | GGTTACCCCATGGAACTTA | 249 |
| GTCTGCAGGACAATTCAT | 154 | CAGGAAGCTATCCCTATTCT | 250 |
| CCTAGTAGAATGTTTACTACCAA | 1 | CTGCTTCTTGAGTAACACTT | 2 |
| CATGTTCATGCTGTGTATGT | 3 | GCTTCTTTACAAACGTTCAGAA | 4 |
| TCTATGTTCGAACAGGTATCT | 5 | ACTGCTAAACACTAATATAACCTT | 6 |
| | | TG | |
| TTGAAATGTGTTTTATAATTTAGACT | 7 | CCATGAGGTACTGGCC | 8 |
| AGT | | | |
| TTGGTGTTACTGGATCAAATC | 9 | TGCTGAACCAGTCAAACT | 10 |
| TATTATTTTATTTTACAGAGTAACAG | 11 | TTTAGCACTTACCTGTGACT | 12 |
| ACTAG | | | |
| TGGAATGCCAGAACTACA | 13 | GTGGAAGATCCAATCCATTTT | 14 |
| GGAATGAATGGCTGAATTATG | 15 | GCGGTATAATCAGGAGTTTT | 16 |
| AGTTGGCCTGAATCACTATA | 17 | GATGTTACTATTGTGACGATCTC | 18 |
| GTGGTCACTAAACCTTAAGA | 45 | GGCTTACCTTAGTGTAAGAG | 46 |
| TTTCATCGAGATGGGAAATATG | 47 | ACCTGTTGGTATTTGGATACT | 48 |
| AGAAGATAATATTGAAGCTGTAGG | 49 | AGAACTCTTATTTTTTAATCTGATT | 50 |
| | | TTCA | |
| GGACAGCTATTGAAGCATTTA | 51 | CACAAGAACAAGGGAAACAC | 52 |
| GCAGGCAGCTGAGTATC | 53 | TCATCCTGAATTGTAGCAATCA | 54 |
| TCCACAAAGCCCCTTATAAT | 155 | GTTACCCCAACGGCTAC | 251 |
| CTTGTTTCAGGCATGTAGT | 156 | TGCAGATGGCATCATTAATC | 252 |
| CCCACGCCGTCTTA | 157 | CACCCAGAAAGCAGACTA | 253 |
| AGCGGTGGTGTAGTAC | 158 | CGACACACACGACAATAC | 254 |
| AAGTGCCTTAGCAGAGA | 159 | GTCACAGCCTTCTTCATG | 255 |
| AGACATCAGAAAGCATGATC | 160 | CGCATGGCCTCTTCT | 256 |
| TTTGATTGCTGCATATTTCAG | 71 | TCAAAGCATTCTTACCTTACTAC | 72 |
| TTTTAAACTTTTCTTTTAGTTGTGC | 73 | ACTCGATAATCTGGATGACT | 74 |
| CAATTTAGTGAAATAACTATAATGG | 75 | AGTGCCACTGGTCTATAAT | 76 |
| AAC | | | |
| CCTGTGAAATAATACTGGTATGT | 77 | CTACTTTGATATCACCACACAC | 78 |
| TAGAGCGTGCAGATAATGA | 79 | TCAACAACCCCCACAAA | 80 |
| CTTTCTCTAGGTGAAGCTGTA | 81 | GGTTCATTCTCTGGATCAGA | 82 |
| CCATGAGGCAGAGCATA | 161 | GCACCGAGACGATGAA | 257 |
| GGCCATGGCCTGAC | 162 | GCACCTGGCTCCTCT | 258 |
| TATGGTCATGGAAGGGG | 163 | CCCCATACAATTTGATGACA | 259 |
| CCGCTGAGCCACT | 164 | TCCGCCGCACTTAC | 260 |
| GCGTCATCATCTTTGTCAT | 165 | GTGAGCAGGTGGAAGTAG | 261 |
| CATCCCTGACTGTGAGAT | 166 | CCAGATGAGCAGCGT | 262 |
| GGTGGCATGGACAGA | 167 | TCTTCCTCCTCTTCTTCTTC | 263 |
| GTGGGCTACAAGAACTAC | 168 | CAGCAAGTGCCCAGTA | 264 |
| GGGTATGGACACGTTCAT | 169 | CCGAGTCCAGCACCTC | 265 |
| CCTCGAAATGAAGCTACAAC | 170 | AGGCTCCCACCTTTCA | 266 |
| AAGTGGAGAATGTCAGTCT | 171 | ACAGACCCTCTCACTCAT | 267 |
| GAAACTTTCCACTTGATAAGAG | 172 | CCAAGGGTGCAGTTATG | 268 |
| TTACCTCGCTTAGTGCT | 173 | TGCCTCTTGCTTCTCTT | 269 |
| CCCAGAGACCCCAGT | 174 | CCTCACTGATTGCTCTTAGG | 270 |
| AGCCCTGTCGTCTCT | 175 | GCAGCTGTGGGTTGAT | 271 |
| GCCAGGCATTGAAGTC | 176 | GGCCCCTGTCATCTTC | 272 |
| GAGCTGCTGGTGCA | 177 | GTCCTCTGACTGCTCTTT | 273 |
| TCCAATGGATCCACTCAC | 178 | GCTGGATCCCCACTTTT | 274 |

In some embodiments of the method, the plurality of amplicons are generated by at least two, at least three, at least four, at least five, at least ten, at least twenty, at least thirty, at least forty, at least fifty, or at least one hundred or more pairs of primers disclosed in Table 2.

TABLE 2
Primer Pair Mix Pool 2

| | SEQ ID | | SEQ ID |
|---|---|---|---|
| Forward Primer Sequence | NO. | Reverse Primer Sequence | NO. |
| TCACGTTGGTCCACATC | 275 | GGTCTGACGGGTAGAGT | 370 |
| ACAGGATGACAGGAAGAG | 276 | TCTGTCTCCCCACAGAG | 371 |
| ACGATTTCCCTTGGAGATAT | 277 | TGAAGTGTGCTCTGAACA | 372 |
| AGACAACTGTTCAAACTGAT | 278 | ATGCTTGCTCTGATAGGA | 373 |
| GACCCCAAGCTTTAGTAAAT | 279 | ACACAAGACTCAGAATAGATACA | 374 |
| ACACCCTCCATTTTATCAC | 280 | ATGTCTCTTCTTCCTACCTG | 375 |
| CACGAATGTGTGGTTAACTC | 281 | GCCACACGTGGTATTCA | 376 |
| TTTCCTTCCCCCTTGTC | 282 | TGAGCTCCCTACCTGATT | 377 |
| TTGTATTCTCTGCCTTCTCT | 283 | CACCTTCTCCAGCATTTTC | 378 |
| AATAACCGCTCCTCATCA | 284 | CATCATCCTCACCTGACTC | 379 |
| CTGAGACTAGATGACTTTTGTC | 285 | TTCAAAATGTAGACCACAGAC | 380 |
| CTTTACTTAAATAGGGCAAGTTC | 286 | CGGAAGAACTCTCCAGTATTC | 381 |
| TTCCTTTATTTTTGTTCCCAAAG | 287 | CCTTGTTGAAGGAGCAGAA | 382 |
| CAACGCCACAACCAC | 288 | CCAGAGCCCGACTCG | 383 |
| GTACCAGATGGATGTGAAC | 289 | TCTCCAAGATGGGATACTC | 384 |
| AGAGACATGCATGAACATTT | 290 | GGCGTCTGCGTACTTC | 385 |
| CTTGTCTCTGTGTTCTTGT | 291 | CAGGGACCTTACCTTATACA | 386 |
| AGTTAACGTCTTCCTTCTCT | 292 | CCCCCACACAGCAAA | 387 |
| GCATCTGCCTCACCT | 293 | GTATCTCCCTTCCCTGATTAC | 388 |
| AGCCAGGAACGTACTG | 294 | TGGTCCCTGGTGTCA | 389 |
| AAGGGATTGTGATTGTTCAT | 295 | AGGCGTTCTCCTTTCTC | 390 |
| CCTGATCTCCTTAGACAACT | 296 | GCAGTGAGTGGGTACCTC | 391 |
| ATACCCTCTCAGCGTAC | 297 | CCGGACATGGTCTAAGAG | 392 |
| GGGGATGAGCTACCTG | 298 | CCATCTGCATGGTACTCT | 393 |
| TTGGCCAGCAAGAATG | 299 | GTGCTCTCATTTTAAAGATGG | 394 |
| CAGTGCAAGGTTTACACA | 300 | GCTTGTTTGCTGAATGTTAAC | 395 |
| GAAAGGAGAGCAGGATAATAA | 301 | GTGTGCAGAACAATGTGA | 396 |
| CACTGATATTTAAATGCCTTAGAG | 302 | GGACTTCAAGAACTTGGATTA | 397 |
| CAGCAGCTTGGTTTCTTC | 303 | ACTATGGGACTTGAAAACGG | 398 |
| TGCAACGTGTGTAGACA | 304 | ACAGGCCAGTGTTTACAT | 399 |
| TCCCAACCATGACAAGA | 305 | AACATCATCATTAGTGGATCTAC | 400 |
| GCTACTTGCAATGATATACAC | 306 | TGTGATCACATGCTTACAGT | 401 |
| CACCCAATGAAGAATGTAATTG | 307 | GAGAAATTGCTTGCTTTAGATG | 402 |
| AGCCCAGCCATTTCTAA | 308 | GTGTCAGAGATGGAGATGAT | 403 |
| CTGGCTGCTGAAGTCT | 309 | AGCGCCTGGAAGAGA | 404 |
| CACTGTGGAGGCATTTG | 310 | GGCATGAGGTCACTGAC | 405 |
| CAGTGGCTCAAGCAC | 311 | GCTCCAACCCCTAGAC | 406 |
| GAGGAGCCCGTGTC | 312 | ACACCAGGTCCTTGAAG | 407 |
| CTGCAGCTGGTCCTT | 313 | GGGATGCCACTCACAG | 408 |
| GCAGGAGCTGACAGTA | 314 | GGAAGCTGGCAATCTCTA | 409 |
| CGGTAGTTGCCCTTCTC | 315 | CCCGTTCTACGAGAAGAATAA | 410 |
| CGCTGTGTCCTTTCAG | 316 | GCCCACCTCGTTGT | 411 |
| GACATTTTCAAAGCAGTGTATC | 317 | CCCTAAGTTTGTAAGTAGTGC | 412 |
| CCAGTCCCTCTGGAATAA | 318 | AGAAGCAAAGCGTTCTTTAC | 413 |
| CTCTATAGTGGGGTCGTAT | 319 | AGACCCTGTAGGAGGAC | 414 |
| ACATTATTGCCAACATGACT | 320 | TGGCACCATACGAAATATTC | 415 |
| CATACTCAACACGATTCTGT | 321 | CCGGATCAGTGCATAACA | 416 |
| GGCACGGTTGAATGTAA | 322 | ATCATGACTGATATGGTAGACA | 417 |
| CCACATTTCTCTTCCATTGTA | 323 | TCTGGAGAGAGAACAAATAAATG | 418 |
| AGACAATAATTATTAAAAGGTGATC | 324 | ACTCAGCCTGTTTCTGG | 419 |
| TATT | | | |
| TTAGCGAGTGCCCATT | 325 | CCTGACAGACAATAAAAGGC | 420 |
| TGTCCCACTTGATTCAGT | 326 | GCTTGATAGGTAGGTACTCA | 421 |
| CGTGAGTACCCATTCTCT | 327 | CATGCAAATTTTGCTGAAGT | 422 |
| CCAAAGACAACTTCATTAGACT | 328 | CCTTCCTTGATCATCTTGTAGA | 423 |
| GTATTTATTTCAGTGTTACTTACCTG | 329 | AGTTAAGGACTCTGAAGATGT | 424 |
| GGATCATATTCGTCCACAAA | 330 | GTGTGACATGTTCTAATATAGTC | 425 |
| | | A | |
| CTTTCTCCAGCTAATTCATCT | 331 | GCTCCATGCAGATACTGA | 426 |
| CTGCAGCCAGAAAGACT | 332 | CCTGGCACCCAAACAT | 427 |
| TCCTCTCGTTTCCTTACAT | 333 | GCTTCCCAAACACTTAGAC | 428 |
| CAAAGCAAGCCAGATTCT | 334 | AAGCAGTGCTCATGATTG | 429 |
| GCACTGGGTCAAAGTCT | 335 | TTGGGCTTACACTTCGG | 430 |
| CAGTGCTAACCAAGTTCTT | 336 | AACCACAAAAGTATACTCCATG | 431 |
| CGCTCCTGGGAATCT | 337 | GGATGGCTGGCTTACA | 432 |
| CAGTCAAGGTTGCTGATT | 338 | CATCTGACTTGGTGGTAAAC | 433 |
| GGCGTCAGGAACTG | 339 | CGGAGCTTCCTGAGTG | 434 |
| CGGCCTCGATCTTGTA | 340 | GCCCCTCTCTGATTGTC | 435 |
| CAGCTCCTCCTCGC | 341 | GGTGGTGGTGCTGATG | 436 |
| GGTGGGATCATATTCATCTAC | 342 | CGCCAATTAACCCTGATTA | 437 |
| GTCTGAACTGAAGATAATGACT | 343 | GGATTTAAGCCTGATTGAACA | 438 |
| TGTCCACCGTGATCTG | 344 | ACCAGTGAGGGAAGTGA | 439 |
| GTAAGTGTTACTCAAGAAGC | 19 | ATAGGATATTGTATCATACCAAT | 20 |
| TCT | | T | |
| TCCACAGCTACACCATATAT | 21 | AGCATCAGCATTTGACTTTA | 22 |
| TACACAGACACTCTAGTATCTG | 23 | GAAGGTTTGACTGCCATAAA | 24 |
| ATGACAAAGAACAGCTCAAA | 25 | GAGATCAGCCAAATTCAGTT | 26 |
| GATGTGTTACAAGGCTTATCTA | 27 | GCCTCTTGCTCAGTTTTATC | 28 |
| GAGGCTTTGGAGTATTTCA | 29 | CTGCTGAGAGTTATTAACAGT | 30 |
| GCTTTTGGAGTCCTATTGT | 31 | CACAAACTAGAGTCACACAC | 32 |
| GGGTTTTGGGCTGATATTA | 33 | CCACAGAACTGAAGGTTAAT | 34 |
| TTATCCATTGAATTTATTTTAATCTT | 35 | GGGATGTGCGGGTATATT | 36 |
| TCTAG | | | |
| GTCTTGCAGTAAGAGATTGT | 37 | TCTTTGCTGTACCGCT | 38 |
| GTTTCTTTTGCCTGCA | 39 | TGGATAAGGTCTGGTTTAATG | 40 |
| GCTACAATTCAGGATGAGTTA | 41 | TCTTCTGCTATCACCATCTTT | 42 |
| CCATCATGATGAGAAGACAT | 43 | TTGCTGGAGATACATACACT | 44 |
| TCGTCTGGGAACTATACTC | 345 | GGTAAATGGACAAGAACACT | 440 |
| TGTACTGCTCCCAGAAGA | 346 | GCCCAGTTCCCTTTCTAC | 441 |
| TGCACCAGGAGTTTGTA | 347 | TCTGGCTGCGAGTTATAAT | 442 |
| GAGGGTGTCCTGTGT | 348 | CAGCTTTGCCCATGAAAC | 443 |
| GCCACAGCAGTCTGAAA | 349 | CCAGGCAGCGGTAGTA | 444 |
| AGGTTCGCTGCTTTTAATC | 350 | GCTACTGGCCGGAAAG | 445 |
| CAGCTTCTGCCATCTCT | 55 | AGCAGCCGCAGAAAT | 56 |
| GTGGCTTTTTGTTTGTTTG | 57 | CACTCTAACAAGCAGATAACT | 58 |
| TACTTGTTAATTAAAAATTCAAGAG | 59 | CTTAGCCATTGGTCAAGATC | 60 |
| TTTT | | | |
| ACAATCATGTTGCAGCA | 61 | AAAAACATCAAAAAATAACTTAC | 62 |
| CTTTT | | | |
| AGAGGCGCTATGTGTATTA | 63 | CATGGAAGGATGAGAATTTCA | 64 |
| GGAAGACAAGTTCATGTACT | 65 | CTGTCCTTATTTTGGATATTTCT | 66 |
| | | C | |
| ATTAATTAAATATGTCATTTCATTTC | 67 | GCTATCGATTTCTTGATCACA | 68 |
| TTTTTC | | | |
| TGAGTCATATTTGTGGGTTTTC | 69 | TGATCAGGTTCATTGTCACTAA | 70 |
| CCCGGGGGATTAAAGC | 351 | TCAGATGTGCTGTTGAGAC | 446 |
| GGTCAGCTACTCCTCTTC | 352 | AGGATGGCCTCTGTCT | 447 |
| CCCCTCCTTCCTAGAGA | 353 | GAGCAACACCCACACTTA | 448 |
| GTGCTGCATTTCAGAGAA | 354 | CAGCTGGCCTTACCAT | 449 |
| GTGCAGAACATCAAGTTCA | 355 | GTAGCTGTGCATGTCCT | 450 |
| TGGCTTTGTGCTCATTAC | 356 | GGAGGTGGGTGTCTTTAT | 451 |
| CTGCTCTCAGGTTGACT | 357 | GTCGTGGGACACAGTG | 452 |
| GAGCTGGGGACTCTT | 358 | GGTGCGGGAGTGAATAG | 453 |
| CAGGAGTCATGACTCTGTT | 359 | GGAGGGTGCAGTGTTG | 454 |
| TGGCCTTTGACCTCAAT | 360 | CTCCAGAAGCTTGAACTCT | 455 |
| GCCCGCAGGTACTT | 361 | CTAGCACGTGCCTACC | 456 |
| ATCCTCCGGCTGAAG | 362 | CCTGAGTGTAGATGATGTCA | 457 |
| CTGGAGTGAGCCCTG | 363 | ACTCAGGTACTGTGTATATACTT | 458 |
| GCAGTGCTAGGAAAGAG | 364 | GAAGAGAATCTCCGCAAG | 459 |
| TGCAGGGTGGCAA | 365 | GCACTGGCCTCATCTTG | 460 |
| CGTCATGTGCTGTGAC | 366 | GCCCTGACTTTCAACTCT | 461 |
| GTGTTTCTGTCATCCAAATAC | 367 | AGCGCTGCTCAGATAG | 462 |
| TGCCCTGGTAGGTTTTC | 368 | ACCCAGGTCCAGATGAA | 463 |
| CAAGGGGGACTGTAGAT | 369 | ACTGACTTTCTGCTCTTGT | 464 |

Another aspect of the methods of the present technology is that the 231 amplicons generated by their respective forward and reverse primer pairs shown in Tables 1 and 2 map to genes or gene regions not typically tested in a given tumor type. For example, typically tested genes or gene regions include EGFR exons 18-21, KRAS exons 1-2, and ALK translocations for lung cancer; KRAS/NRAS exons 1-2, and PIK3CA exons 9 and 20 for colorectal cancer; BRAF exon 15, and KIT for melanoma; and KIT and PDGFRa for gastrointestinal stromal tumors.

In some embodiments, a single primer or one or both primers of a primer pair comprise a specific adapter sequence (also referred to as a sequencing adapter) ligated to the 5′ end of the target specific sequence portion of the primer. This sequencing adapter is a short oligonucleotide of known sequence that can provide a priming site for both amplification and sequencing of the adjoining, unknown target nucleic acid. As such, adapters allow binding of a fragment to a flow cell for next generation sequencing. Any adapter sequence may be included in a primer used in the present technology. In certain embodiments, amplicons corresponding to specific regions of AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN are amplified using primers that contain an oligonucleotide sequencing adapter to produce adapter tagged amplicons.

In other embodiments, the employed primers do not contain adapter sequences and the amplicons produced are subsequently (i.e. after amplification) ligated to an oligonucleotide sequencing adapter on one or both ends of the amplicons. In some embodiments, all forward amplicons (i.e., amplicons extended from forward primers that hybridized with antisense strands of a target nucleic acid) contain the same adapter sequence. In some embodiments when double stranded sequencing is performed, all forward amplicons contain the same adapter sequence and all reverse amplicons (i.e., amplicons extended from reverse primers that hybridized with sense strands of a target segment) contain an adapter sequence that is different from the adapter sequence of the forward amplicons. In some embodiments, the adapter sequences further comprise an index sequence (also referred to as an index tag, a “barcode” or a multiplex identifier (MID)).

In some embodiments, the adapter sequences are P5 and/or P7 adapter sequences that are recommended for Illumina sequencers (MiSeq and HiSeq). See, e.g., Williams-Carrier et al., Plant J., 63(1): 167-77 (2010). In some embodiments, the adapter sequences are P1, A, or Ion Xpress™ barcode adapter sequences that are recommended for Life Technologies sequencers. Other adapter sequences are known in the art. Some manufacturers recommend specific adapter sequences for use with the particular sequencing technology and machinery that they offer.

Additionally or alternatively, in some embodiments of the above methods, amplicons corresponding to specific regions of AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN from more than one sample are sequenced. In some embodiments, all samples are sequenced simultaneously in parallel.

In some embodiments of the above methods, amplicons corresponding to specific regions of AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN from at least 1, 5, 10, 20, 30 or up to 35, 40, 45, 48 or 50 different samples are amplified and sequenced using the methods described herein.

Additionally or alternatively, in some embodiments of the method, amplicons derived from a single sample may further comprise an identical index sequence that indicates the source from which the amplicon is generated, the index sequence for each sample being different from the index sequences from all other samples. As such, the use of index sequences permits multiple samples to be pooled per sequencing run and the sample source subsequently ascertained based on the index sequence. In some embodiments, the Access Array™ System (Fluidigm Corp., San Francisco, CA) or the Apollo 324 System (Wafergen Biosystems, Fremont, CA) is used to generate a barcoded (indexed) amplicon library by simultaneously amplifying the nucleic acids from the samples in one set up.

In some embodiments, indexed amplicons are generated using primers (for example, forward primers and/or reverse primers) containing the index sequence. Such indexed primers may be included during library preparation as a “barcoding” tool to identify specific amplicons as originating from a particular sample source. When adapter-ligated and/or indexed primers are employed, the adapter sequence and/or index sequence gets incorporated into the amplicon (along with the target-specific primer sequence) during amplification. Therefore, the resulting amplicons are sequencing-competent and do not require the traditional library preparation protocol. Moreover, the presence of the index tag permits the differentiation of sequences from multiple sample sources.

In some embodiments, the amplicons may be amplified with non-adapter-ligated and/or non-indexed primers and a sequencing adapter and/or an index sequence may be subsequently ligated to one or both ends of each of the resulting amplicons. In some embodiments, the amplicon library is generated using a multiplexed PCR approach.

Indexed amplicons from more than one sample source are quantified individually and then pooled prior to high throughput sequencing. As such, the use of index sequences permits multiple samples (i.e., samples from more than one sample source) to be pooled per sequencing run and the sample source subsequently ascertained based on the index sequence. “Multiplexing” is the pooling of multiple adapter-tagged and indexed libraries into a single sequencing run. When indexed primer sets are used, this capability can be exploited for comparative studies. In some embodiments, amplicon libraries from up to 48 separate sources are pooled prior to sequencing.

Following the production of an adapter tagged and, optionally indexed, amplicon library, the amplicons are sequenced using high throughput, massively parallel sequencing (i.e., next generation sequencing). Methods for performing high throughput, massively parallel sequencing are known in the art. In some embodiments of the method, the high throughput massive parallel sequencing is performed using 454TM GS FLX™ pyrosequencing, reversible dye-terminator sequencing, SOLID sequencing, Ion semiconductor sequencing, Helioscope single molecule sequencing, sequencing by synthesis, sequencing by ligation, or SMRT™ sequencing. In some embodiments, high throughput massively parallel sequencing may be performed using a read depth approach.

In some embodiments, the methods of the present technology are useful in detecting at least one mutation in amplicons corresponding to specific regions of AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN in a FFPE tumor sample comprising a heterogeneous tumor. In certain embodiments, 5% of the cells of the heterogeneous tumor harbor at least one mutation in at least one of the plurality of amplicons. In some embodiments, about 10% of the cells of the heterogeneous tumor harbor at least one mutation in at least one of the plurality of amplicons. In some embodiments, about 25% of the cells of the heterogeneous tumor harbor at least one mutation in at least one of the plurality of amplicons. In some embodiments, about 50% of the cells of the heterogeneous tumor harbor at least one mutation in at least one of the plurality of amplicons. In other embodiments, at least 5% of the cells of the heterogeneous tumor harbor at least one mutation in at least one of the plurality of amplicons. In other embodiments, at least 10% of the cells of the heterogeneous tumor harbor at least one mutation in at least one of the plurality of amplicons. In other embodiments, at least 25% of the cells of the heterogeneous tumor harbor at least one mutation in at least one of the plurality of amplicons.

### Treatment of Solid Tumors

Disclosed herein are methods for determining whether a patient diagnosed with breast cancer, colorectal cancer, melanoma or lung cancer will benefit from or is predicted to be responsive to treatment with an individual therapeutic agent or a specific combination of therapeutic agents.

In some embodiments, the therapeutic agent(s) comprise one or more of anti-HER-2 therapies, anti-EGFR therapies, P13K/AKT/mTor pathway inhibitors, kinase inhibitors, Notch pathway inhibitors, BRAF inhibitors, SMO antagonists, ALK/MET inhibitors, ERBB2 antagonists, FGFR3 antagonists, and RAF/MEK/ERK inhibitors.

In certain embodiments, the EGFR tyrosine kinase inhibitor is gefitinib or erlotinib. In certain embodiments, the anti-EGFR therapy is cetuximab.

In some embodiments of the method, the anti-HER-2 therapy is trastuzumab or lapatinib.

Examples of kinase inhibitors include but are not limited to crizotinib, afatinib, Axitinib, bevacizumab, Bosutinib, Cetuximab, Dasatinib, Erlotinib, Fostamatinib, Gefitinib, Imatinib, Lapatinib, Lenvatinib, Nilotinib, Panitumumab, Pazopanib, Pegaptanib, Ranibizumab, Ruxolitinib, Sorafenib, Sunitinib, Trastuzumab, and Vemurafenib.

Examples of BRAF inhibitors include, but are not limited to GDC-0879, SB590885, Encorafenib, RAF265, TAK-632, PLX4720, CEP-32496, AZ628, Sorafenib Tosylate, Sorafenib, Vemurafenib (Zelboraf) and Dabrafenib (GSK2118436).

Examples of RAF/MEK/ERK inhibitors include, but are not limited to Vemurafenib (Zelboraf) and Dabrafenib (GSK2118436), Encorafenib, TAK-632, PLX4720, MLN2480, Cobimetinib (GDC-0973), MEK 162, RO5126766, GDC-0623, VTX11e, Selumetinib (AZD6244), PD0325901, Trametinib (GSK1120212), U0126-EtOH, PD184352 (CI-1040), Refamctinib, PD98059, BIX02189, Binimetinib, Pimasertib (AS-703026), SL327, BIX02188, AZD8330, TAK-733, PD318088, SCH772984, and FR 180204.

Examples of P13K/AKT/mTor pathway inhibitors include, but are not limited to BKM120, BEZ235, Pictilisib (GDC-0941), LY294002, CAL-101 (Idelalisib), GNE-317, PI-3065, HS-173, PI-103, NU7441, GSK2636771, VS-5584, CZC24832, Duvelisib, TG100-115, A66, YM201636, CAY10505, GSK1059615, PF-04691502, PIK-75, PIK-93, AS-605240, BGT226, AZD6482, Voxtalisib, Alpelisib, CUDC-907, IC-87114, Omipalisib, TG100713, Gedatolisib, CH5132799, PKI-402, BAY 80-6946, TGX-221, XL147, PIK-90, PIK-293, PIK-294, 3-Methyladenine, Quercetin, Wortmannin, ZSTK474, AS-252424, AS-604850, everolimus, and Apitolisib.

Examples of Notch pathway inhibitors include, but are not limited to FLI-06, LY411575, Dibenzazepine, RO4929097, Compound E, Z-Leu-Leu-Nle-CHO, SAHM1, TR4 and Semagacestat.

Examples of SMO antagonists include, but are not limited to Purmorphamine, Taladegib (LY2940680), Cyclopamine, Vismodegib (GDC-0449), LDE225, Glasdegib (PF-04449913), PF-5274857, TAK-441, SANT-1, BMS-833923, GANT61 and IPI-926.

Examples of ERBB2 antagonists include, but are not limited to Lapatinib, Canertinib, CP-724,714, AZD8931, AEE788, Tyrphostin AG 879, Mubritinib, and Pertuzumab.

Examples of FGFR3 antagonists include, but are not limited to BGJ398 (NVP-BGJ398), AZD4547, LY2874455, Dovitinib Dilactic acid, Dovitinib, Dovitinib Lactate, CH5183284, and Nintedanib.

Examples of ALK inhibitors include, but are not limited to Crizotinib, TAE684, Alectinib, Ceritinib, AP26113, AZD3463, and ASP3026.

Examples of MET inhibitors include, but are not limited to Crizotinib, PHA-665752, SU11274, SGX-523, BMS-777607, JNJ-38877605, Tivantinib, PF-04217903, MGCD-265, Capmatinib, AMG 208, MK-2461, AMG 458, NVP-BVU972, and Tepotinib.

The BRAF inhibitor vemurafenib is the most common targeted therapy for melanoma and is used treat BRAF V600 mutation-positive tumors. In addition, sequencing for KIT mutations may also be performed to assess the likely effectiveness of treatment with imatinib mesylate. Thus, in some embodiments, the present disclosure provides methods for determining whether a melanoma patient is likely to respond to treatment with vemurafenib or MEK inhibitors. Also provided herein are methods for determining whether a melanoma patient is likely to respond to treatment with a drug that inhibits PIK3CA or MET activity.

Colorectal cancer specimens are generally submitted for detection of mutations in genes downstream of EGFR to identify tumors with a low likelihood of response to anti-EGFR therapies such as cetuximab. NCCN guidelines identify mutations in the KRAS, NRAS, or BRAF as indicators of lack of responsiveness to such therapies, and clinical evidence suggests that PIK3CA and PTEN mutations are also indicators of resistance. Sec Er T. et al., BioMed Research International 2014:1-8 (2014); Bokemeyer et al., Ann Oncol. 22(7):1535-46 (2011). Thus, in some embodiments, the present disclosure provides methods for determining whether a colorectal cancer patient is likely to respond to treatment with anti-EGFR therapy (e.g., cetuximab).

For lung cancer specimens, the current guideline-recommended testing (CAP, IASL, AMP) is to prioritize testing for EGFR mutations, which directly identifies EGFR TKI responders. These guidelines also state that EGFR mutation-negative specimens should be followed up with ALK rearrangement testing to identify likely responders to the ALK/c-MET inhibitor crizotinib. KRAS mutation testing may be used as a means to identify non-responders to EGFR tyrosine kinase inhibitors such as gefitinib and erlotinib. However, this does not rule-in a TKI responder, as EGFR tyrosine kinase inhibition by these drugs is specific to tumors harboring tyrosine kinase domain mutations in EGFR. In some embodiments, the present disclosure provides methods for determining whether a lung cancer patient is likely to respond to treatment with an EGFR TKI. In certain embodiments, the EGFR tyrosine kinase inhibitor is gefitinib or erlotinib. Also provided herein are methods for determining whether a lung cancer patient is likely to respond to treatment with crizotinib. The present disclosure also provides methods for determining whether a lung cancer patient is likely to respond to treatment with vemurafenib, dabrafenib, dasatinib or a MEK inhibitor such as selumetinib.

The primary routine clinical biomarkers for targeted treatment in breast cancer are HER-2 gene amplification/expression (FISH and/or IHC) for anti-HER-2 therapy (e.g., trastuzumab) and ER/PR overexpression for endocrine therapy. In some embodiments, the present disclosure provides methods for determining whether a HER-2 positive patient diagnosed with breast cancer is likely to respond to treatment with anti-HER-2 therapies or trastuzumab emtansine. In some embodiments of the method, the anti-HER-2 therapy is trastuzumab or lapatinib. Also provided herein are methods for determining whether a patient diagnosed with breast cancer is likely to respond to treatment with P13K/AKT/mTOR pathway inhibitors.

In another aspect, the present disclosure provides a method for selecting a subject for treatment with a P13K/AKT/mTor pathway inhibitor and at least one additional agent comprising (a) extracting genomic DNA from a formalin fixed paraffin-embedded specimen obtained from the subject; (b) generating a library comprising amplicons corresponding to each of a plurality of cancer-related genes, said plurality of cancer-related genes comprising AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN, wherein (i) generating said library proceeds independently of using a bait set comprising nucleic acid sequences that are complementary to at least one of the plurality of amplicons, and (ii) the quality of the genomic DNA extracted from the formalin fixed paraffin-embedded specimen is not assessed using quantitative PCR prior to generating the library; (c) detecting at least one mutation in at least one of the plurality of amplicons; and (d) selecting the subject for treatment with a P13K/AKT/mTor pathway inhibitor and at least one additional agent, if a mutation in at least one of the amplicons corresponding to PIK3CA, PIK3R1 and PTEN, and a mutation in at least one of the amplicons corresponding to NOTCH1, ERBB2, BRAF, PTCH1, SMO, EGFR, KRAS, DDR2, MAP2K1, FGFR3, NRAS, MET, and FBXW7 are detected.

In some embodiments of the method, the at least one additional agent is selected from Notch pathway inhibitors, BRAF inhibitors, SMO antagonists, MET inhibitors, ERBB2 antagonists, or any combination thereof. In some embodiments of the method, the at least one additional agent is selected from Notch pathway inhibitors, FGFR3 antagonists, RAF/MEK/ERK inhibitors, or any combination thereof. In some embodiments, the at least one additional agent is RAF/MEK/ERK inhibitors, FGFR3 antagonists, SMO antagonists or any combination thereof.

In another aspect, the present disclosure provides a method for selecting a subject for treatment with a EGFR tyrosine kinase inhibitor and at least one additional agent comprising: (a) extracting genomic DNA from a formalin fixed paraffin-embedded specimen obtained from the subject; (b) generating a library comprising amplicons corresponding to each of a plurality of cancer-related genes, said plurality of cancer-related genes comprising AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN, wherein (i) generating said library proceeds independently of using a bait set comprising nucleic acid sequences that are complementary to at least one of the plurality of amplicons, and (ii) the quality of the genomic DNA extracted from the formalin fixed paraffin-embedded specimen is not assessed using quantitative PCR prior to generating the library; (c) detecting at least one mutation in at least one of the plurality of amplicons; and (d) selecting the subject for treatment with a EGFR tyrosine kinase inhibitor and at least one additional agent, if a mutation in at least one of the amplicons corresponding to EGFR, and a mutation in at least one of the amplicons corresponding to KRAS, PIK3R1 and BRAF are detected.

In some embodiments of the method, the at least one additional agent is selected from BRAF inhibitors, RAF/MEK/ERK inhibitors, P13K/AKT/mTor pathway inhibitors or any combination thereof.

### Kits

The present disclosure also provides kits for detecting alterations in target nucleic acid sequences corresponding to the preselected set of cancer-related genes described herein.

Kits of the present technology comprise one or more primer pairs that selectively hybridize and are useful in amplifying one or more of the genes selected from the group consisting of AKT1, ERBB2, FOXL2, IDH2, NRAS, RET. ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN.

In some embodiments, the kits of the present technology comprise a single primer pair that hybridizes to a region or exon of a single gene selected from the group consisting of AKT1. ERBB2, FOXL2, IDH2. NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN. In other embodiments, the kits of the present technology comprise multiple primer pairs that hybridize to one or more regions or exons of a single gene selected from the group consisting of AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2. GNAS, MAP2K1, PIK3R1, TP53, DDR2, FGFR3, HRAS, MET, PTCH1, EGFR, FGFR4, IDH1, NOTCH1, and PTEN. In certain embodiments, the kits of the present technology comprise multiple primer pairs comprising a single primer pair that specifically hybridizes to a region or exon of a single gene for each of AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN. In certain embodiments, the kits of the present technology comprise multiple primer pairs comprising more than one primer pair that hybridizes to one or more regions or exons for each of AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN.

Thus, it is contemplated herein that the kits of the present technology can comprise primer pairs that recognize and specifically hybridize to one or more regions or exons of one or more genes selected from the group consisting AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN. Alternatively, the kit can comprise primer pairs that will detect one or more mutations selected from the group consisting of BRAF V600E, BRAF V600K, BRAF K483Q, BRAF G466V, BRAF G464V, BRAF E501V, BRAF E501K, EGFR ΔE746_A750, EGFR R680Q, EGFR G598E, KRAS A146T, KRAS R68M, KRAS L19F, KRAS G12V, KRAS G12D, KRAS G12C, KRAS G13D, KRAS G13C, KRAS G12A, KRAS G12S, KRAS Q22K, NRAS Q61K, NRAS Q61R, NRAS G12R, NRAS G12D, PIK3CA C420R, PIK3CA G106R, PIK3CA R38H, PIK3CA E453K, PIK3CA H1044R, PIK3CA N1044K, PIK3CA E545K, PIK3CA Q546H, PIK3CA H1047R, PIK3CA H1043L, PIK3CA M1043V, PIK3CA E542K, PIK3CA E542Q, PIK3CA T1053A, PIK3CA 1121V, PIK3CA H1047L, ERBB2 L755S, ERBB2 S310Y, ERBB2 D769Y, ERBB2 S255R, DDR2 H92Y, DDR2 R31L, DDR2 L34P, DDR2 P381R and DDR2 K392N.

In some embodiments, the kits comprise one or more primer pairs disclosed in Table 1. In some embodiments, the kits comprise one or more primer pairs disclosed in Table 2. In some embodiments, the kits comprise two or more primer pairs disclosed in Table 1 and/or Table 2.

In some embodiments, the kits further comprise buffers, enzymes having polymerase activity, enzymes having polymerase activity and lacking 5′→3′ exonuclease activity or both 5′→3′ and 3′→5′ exonuclease activity, enzyme cofactors such as magnesium or manganese, salts, chain extension nucleotides such as deoxynucleoside triphosphates (dNTPs), modified dNTPs, nuclease-resistant dNTPs or labeled dNTPs, necessary to carry out an assay or reaction, such as amplification and/or detection of alterations in target nucleic acid sequences corresponding to the specific set of cancer-related genes disclosed herein.

In one embodiment, the kits of the present technology further comprise a positive control nucleic acid sequence and a negative control nucleic acid sequence to ensure the integrity of the assay during experimental runs. A kit may further contain a means for comparing the levels and/or activity of one or more of the preselected set of cancer-related genes described herein in a tumor sample with a reference nucleic acid sample (e.g., a non-tumor sample). The kit may also comprise instructions for use, software for automated analysis, containers, packages such as packaging intended for commercial sale and the like.

The kits of the present technology can also include other necessary reagents to perform any of the NGS techniques disclosed herein. For example, the kit may further comprise one or more of: adapter sequences, barcode sequences, reaction tubes, ligases, ligase buffers, wash buffers and/or reagents, hybridization buffers and/or reagents, labeling buffers and/or reagents, and detection means. The buffers and/or reagents are usually optimized for the particular amplification/detection technique for which the kit is intended. Protocols for using these buffers and reagents for performing different steps of the procedure may also be included in the kit.

The kits of the present technology may include components that are used to prepare nucleic acids from a solid tumor test sample for the subsequent amplification and/or detection of alterations in target nucleic acid sequences corresponding to the specific set of cancer-related genes disclosed herein. Such sample preparation components can be used to produce nucleic acid extracts from tissue samples. The test samples used in the above-described methods will vary based on factors such as the assay format, nature of the detection method, and the specific tissues, cells or extracts used as the test sample to be assayed. Methods of extracting nucleic acids from samples are well known in the art and can be readily adapted to obtain a sample that is compatible with the system utilized. Automated sample preparation systems for extracting nucleic acids from a test sample are commercially available, e.g., Roche Molecular Systems' COBAS AmpliPrep System, Qiagen's BioRobot 9600, and Applied Biosystems' PRISM™ 6700 sample preparation system.

### EXAMPLES

### Example 1: Design of the Solid Tumor Screening NGS Assay of the Present Technology

Initial experimentation efforts were directed towards designing a more focused, highly sensitive solid tumor screening assay that could provide a general overview of the clinically relevant mutations in multiple solid tumor types while using extremely small amounts of DNA derived from FFPE tissue samples (˜10 ng). A set of 34 cancer-related genes was carefully selected on the basis of NCCN guideline recommendations, prevalence of somatic mutations in solid tumors, and potential for informing treatment selection in solid tumor patients (Table 3).

TABLE 3
Solid Tumor Screening Panel

AKT1
ALK
BRAF
CTNNB1
DDR2
EGFR
ERBB2
ERBB4
FBXW7
FGFR2
FGFR3
FGFR4
FOXL2
GNA11
GNAQ
GNAS
HRAS
IDH1
IDH2
KIT
KRAS
MAP2K1
MET
NOTCH1
NRAS
PDGFRA
PIK3CA
PIK3R1
PTCH1
PTEN
RET
SMO
STK11
TP53

Of the 34 genes selected, many have mutations with known or potential clinical significance in at least 2 (65%) or 3 (40%) solid tumor types (COSMIC, mycancergenome.org, clinicaltrials.gov). Moreover, nearly 80% of these 34 genes are mutated in at least 1% of melanoma, lung cancer, and colorectal cancer (colorectal cancer) cases. The solid tumor screening panel was designed to assay for mutations in target nucleic acid sequences corresponding to specific regions of the 34 genes listed in Table 3 (instead of every exon of the entire gene). The selection of these particular target nucleic acid sequences (or amplicons) was based on the potential for informing treatment decisions, reported mutation frequency, known hot spots etc.

The use of bait sets to enrich for target nucleic sequences corresponding to specific regions of AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN from FFPE samples is significantly inefficient because capture experiments yield about half the number of reads for the interrogated target nucleic sequences compared to that observed with PCR amplification, thereby compromising the overall sensitivity of the screening assay.

As such, subsequent attempts focused on the development of an NGS screening assay that was wholly based on PCR (i.e., amplicon-based library preparation followed by NGS) in order to detect genetic alterations in amplicons corresponding to specific regions of AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN. One of the technical challenges that arose while developing the multiplex PCR method described herein was the optimal selection of over one hundred primer pairs that simultaneously hybridize and amplify target nucleic acid sequences corresponding to specific regions of the 34 cancer-related genes disclosed herein in a single reaction. Achieving the proper balance of primer pairs was a significant concern because differences in annealing efficiency of different primer pairs result in a strong bias in the amplification of the different amplicons, leading to insufficient coverage of some amplicons in a sample and strongly reducing the sensitivity of the assay. In order to maximize the sequencing capacity, the amplification levels should be similar among all amplicons. Furthermore the presence of a large number of different primers results in a strongly increased risk of primer dimer formation diminishing the possibility of reproducible amplifying small amounts of target nucleic acids. To account for the fragmentation observed with FFPE DNA, amplicons were designed to be 126-183 bp. The optimized set of PCR primer pairs useful in the methods of the present technology are disclosed in Tables 1 and 2.

### Example 2: Reproducibility and Analytical Sensitivity of the Solid Tumor Screening Assay of the Present Technology

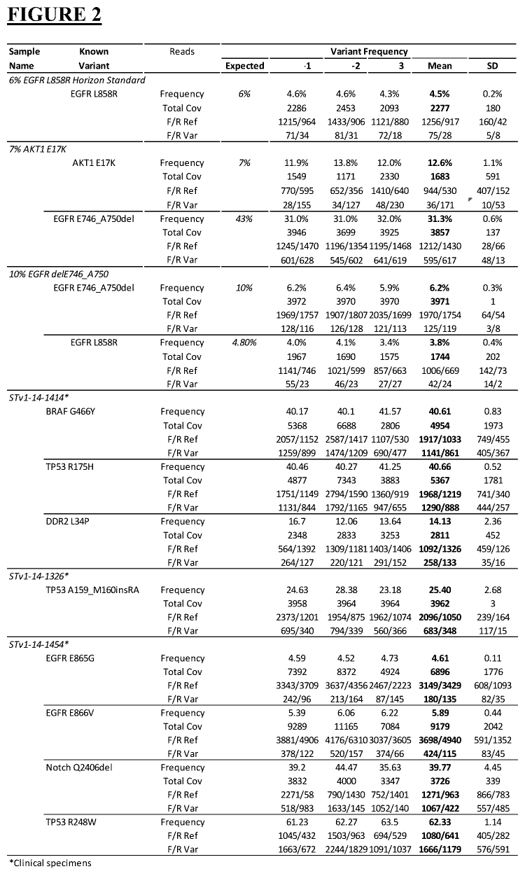

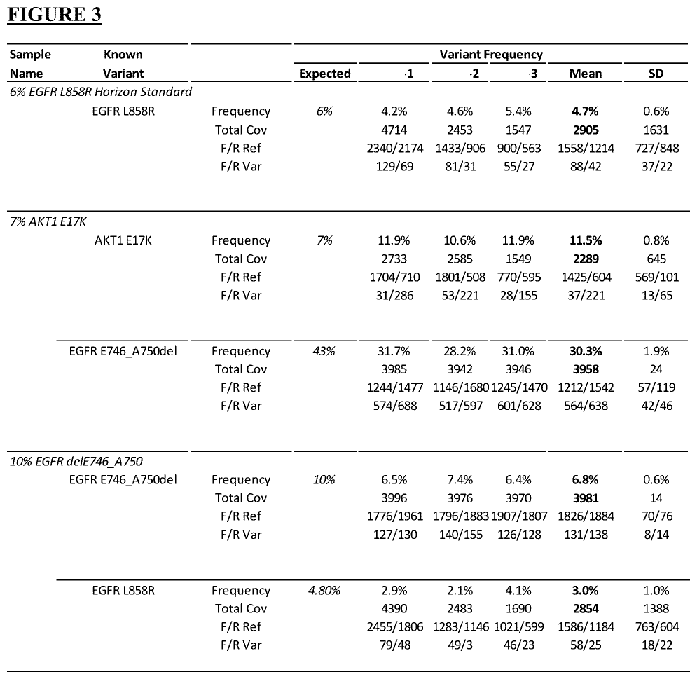

Reproducibility. Inter-assay precision of sequencing performance was assessed by testing three clinical FFPE specimens containing known variants (BRAF G466Y, TP53 R175H, DDR2 L34P, EGFR E865G, EGFR E866V, TP53 R248W, Notch Q2406A, TP53 A159_M160insRA) with mutation frequencies ranging between 4.6% and 62.3% over three different runs. See FIG. 2. For intra-assay precision, three FFPE specimens made from cell lines harboring known variants (2 SNV and 1 DEL) with mutation frequencies ranging between 4.8% and 43% were assayed within a single run. See FIG. 3.

As shown in FIG. 2 and FIG. 3, all expected low-frequency variants were detected in inter- and intra-assay replicates. The SD of variant frequency from inter- and intra-assays for all variants tested ranged from 0.11% to 4.5% and 0.6% to 1.9%, respectively, indicating that the solid tumor screening assay of the present technology is highly reproducible.

Analytical Sensitivity. To demonstrate the assay's analytical sensitivity, clinical FFPE specimens with known mutation frequencies ranging between 14% to 78% were mixed with DNA from FFPE tissue that did not contain mutations in the regions of interest at different ratios. The clinical FFPE specimens were mixed at percentages ranging from 2.5% to 78.0%. See Table 4.

TABLE 4
Tumor Sensitivity Assay

| | | Frequency | | | | |
|---|---|---|---|---|---|---|
| Cell Line Mix | Known Variants | Expected | Observed | Total Cov | F/R Ref | F/R Var |
| CRL-2324D | | | | | | |
| 100% | TP53 R175H | 100% | 99.00 | 1991 | 16/4 | 1189/782 |
| 25% | TP53 R175H | 25% | 36.38 | 1971 | 776/478 | 452/265 |
| 10% | TP53 R175H | 10% | 15.55 | 2000 | 1014/675 | 182/129 |
| 5% | TP53 R175H | 5% | 6.80 | 2000 | 1115/749 | 88/48 |
| 2% | TP53 R175H | 2% | 2.00 | 1998 | 1186/772 | 23/17 |
| 0% | TP53 R175H | 0% | Not detected | | | |
| HTB-126 | | | | | | |
| 100% | PIK3R1 N453_T454insN | 100% | 98.64 | 1984 | 20/7 | 1115/842 |
| 25% | PIK3R1 N453_T454insN | 25% | 8.86 | 1986 | 1020/790 | 94/82 |
| 10% | PIK3R1 N453_T454insN | 10% | 4.97 | 1992 | 1116/777 | 58/41 |
| 5% | PIK3R1 N453_T454insN | 5% | 2.31* | 11891 | 7282/4332 | 275 |
| 2% | PIK3R1 N453_T454insN | 2% | Not detected | | | |
| 0% | PIK3R1 N453_T454insN | 0% | Not detected | | | |
| HTB-126 | | | | | | |
| 100% | TP53 V157F | 100% | 100.00 | 1985 | 0/0 | 1148/837 |
| 25% | TP53 V157F | 25% | 11.18 | 1995 | 1369/403 | 168/55 |
| 10% | TP53 V157F | 10% | 5.71 | 1998 | 1157/727 | 68/46 |
| 5% | TP53 V157F | 5% | 2.86 | 1996 | 1129/810 | 32/25 |
| 2% | TP53 V157F | 2% | Not detected | | | |
| 0% | TP53 V157F | 0% | Not detected | | | |
| HTB-126 | | | | | | |
| 100% | HRAS G12D | 50% | 55.21 | 1391 | 347/276 | 425/343 |
| 25% | HRAS G12D | 12.5% | 3.13 | 1053 | 637/383 | 21/12 |
| 10% | HRAS G12D | 5% | 1.47* | 1157 | 8/9 | 610/530 |
| 5% | HRAS G12D | 2.5% | Not detected | | | |
| 2% | HRAS G12D | 1% | Not detected | | | |
| 0% | HRAS G12D | 0% | Not detected | | | |

*Not detected by variant caller, but visibly present in BAM files and raw read numbers provided (insertion does not break down for/rev reads in IGV.
Not detected, indicates variant present at <2% or completely absent.

Variants in nine target regions were detected, seven of which (2 INDELs and 5 SNPs) were detected in the mixed sample at or near 5% (5%-7%). Variants were not detected in the mixed sample at <2.5%. Although INDELs were detectable in the mixed sample at or near 5%, the observed frequencies of some deletions tended to be lower than expected (2.5-5%). Therefore, based on these data, analytical sensitivity of this assay was defined as 5% for SNPs and 10% for INDELs.

### Example 3: Methods for Validating the Efficacy of the Solid Tumor Screening Assay of the Present Technology with FFPE Samples

This Example demonstrates that the solid tumor screening assay of the present technology has improved breadth of coverage and sensitivity over Sanger sequencing methods and can effectively profile actionable mutations in clinically relevant genes in several major solid tumor types.

Patients and Specimens. This study included de-identified FFPE samples submitted to Quest Diagnostics Nichols Institute, San Juan Capistrano, CA for tumor marker analysis. The diagnosis of each cancer was confirmed by pathology review. A total of 133 FFPE samples were initially collected. Twelve specimens were excluded because of insufficient DNA yield (<10 ng) and 121 tumor specimens (33 colorectal cancer, 27 lung cancer, 31 melanoma and 30 breast cancer) were included in this study.

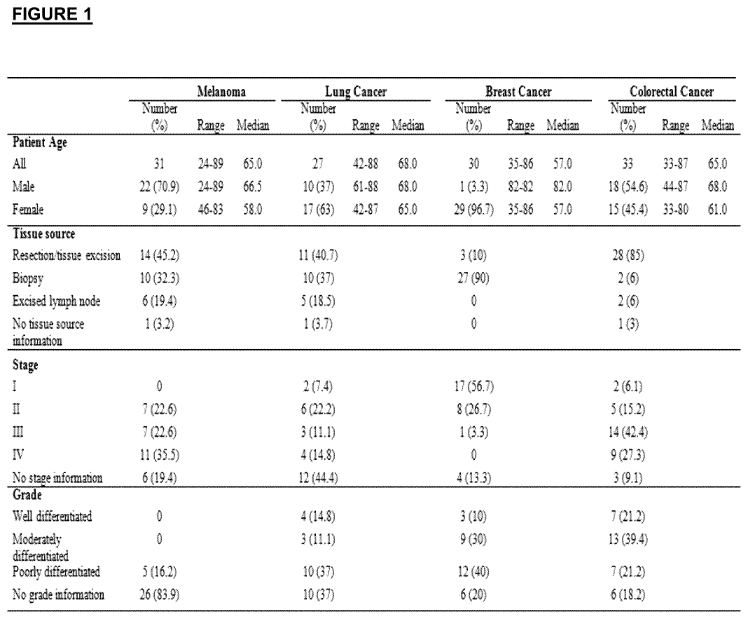

The clinical characteristics of the 121 patients that provided the usable tumor specimens are shown in FIG. 1. The median patient ages for each cancer type ranged between 57 and 68 years. The most common tissue source for melanoma, lung cancer, and colorectal cancer specimens was resection, while most breast cancer specimens originated from biopsies. Stage IV patients accounted for 35.5% and 27.3% of melanoma, and colorectal cancer specimens respectively. Stage I patients accounted for 57% of breast cancer specimens. The most frequent tumor grade designations were “poorly differentiated” in lung cancer (37%) and breast cancer (40%), and “moderately differentiated” in colorectal cancer (39.4%); there was no grading information available for most melanoma specimens (83.9%). See FIG. 1.

DNA Extraction. Hematoxylin and cosin-stained slides for each sample were analyzed by a pathologist to identify tumor-rich areas and estimate tumor fraction. FFPE samples containing >25% tumor cells (based on morphology) in a selected area were included in the study. Sections were subjected to manual macrodissection, and total DNA was extracted from one to five 10-μm unstained sections depending on the tumor area using Roche DNA extraction kit (Roche Molecular Diagnostics, Indianapolis, IN). DNA quantification was performed using a Qubit DNA HS assay kit (Life Technologies, Carlsbad, CA). Samples with at least 10 ng of DNA were selected for NGS analysis. No further evaluation of the quality of the extracted genomic DNA was conducted prior to generating the amplicon-based library (i.e., the quality of the extracted genomic DNA was not assessed using quantitative PCR). This is because earlier comparison studies revealed that there was no significant improvement in using qPCR-based quality control methods prior to performing the NGS-based methods of the present technology compared to Qubit quantitation (data not shown).

Ion Torrent PGM Library Preparation. A PCR amplicon library was generated from the extracted genomic DNA of each sample. Targeted regions within the 34 genes were amplified using 231 primer pairs (listed in Tables 1 and 2) in two primer pools.

Briefly, PCR was performed in 2×10-μL volumes, each containing 5 to 20 ng of genomic DNA; forward and reverse primers listed in either Table 1 or 2 (primer concentrations optimized for balanced amplification); 440 μM (each) dATP, dCTP, dGTP, and dTTP; 5 mM MgCl₂; 57 mM KCl; and 0.6 units of Gold Polymerase (Celera, Alameda, CA). PCR amplification on was carried out on a ABI9700 thermal cycler under the following conditions:

| | | |
|---|---|---|
| Temperature | Time | Cycle |

| | | | | |
|---|---|---|---|---|
| 95° C. | 12 | min | 1 | cycle |
| 95° C. | 15 | sec | 23 | cycles |
| 62° C. | 2 | min | | |

| | | | |
|---|---|---|---|
| 62° C. | 7 | min | 1 |
| 99° C. | 10 | min | 1 |

Sequencing adaptors with short stretches of index sequences (Ion Xpress™ Barcode Adapters 1-16 Kit, Life Technologies, Carlsbad, CA) that enabled sample multiplexing were ligated to the amplicons using the PrepX PGM DNA library kit on the Apollo 324 system (Wafergen Biosystems, Fremont, CA). The amplicon library was then nick-translated using Platinum PCR SuperMix High Fidelity under the following conditions: 1 cycle at 72° C. for 20 minutes and 1 cycle at 95° C. for 5 minutes. The library was quantified using the Qubit DNA HS assay kit (Life Technologies, Carlsbad, CA).

Sequencing Template Preparation. Pooled libraries were created by diluting four patient samples (each with distinct barcoding as described above) to balance the concentrations and generate a final working library concentration of 10 pmol/L. Each library pool contained a positive control DNA sample, harboring 8 variants with known frequencies (Horizon Diagnostics, Waterbeach, Cambridge, UK). Each library pool was subjected to emulsion PCR (E-PCR) using an Ion OneTouch 2 template kit on an Ion One-Touch 2 system (Life Technologies, Carlsbad, CA), according to the manufacturer's protocol. The Qubit Ion Sphere quality control kit (Life Technologies, Carlsbad, CA) was used to estimate the percentage of the Ion Sphere particles (ISPs) with amplified template DNA. Enrichment of ISPs was achieved using the Ion OneTouch kit on the IT OneTouch ES (Life Technologies, Carlsbad, CA) according to the manufacturer's protocol.

Sequencing. Enriched ISPs were subjected to sequencing on an Ion 318 Chip using the Ion PGM sequencing kit (Life Technologies, Carlsbad, CA) according to the manufacturer's instructions.

Data Analysis. The sequence reads from the Ion Torrent PGM library were aligned using the Ion Torrent Suite software version 3.4 (Life Technologies, Carlsbad, CA). The variations were called using the Ion Torrent variant caller using a customized parameter set. Metrics were calculated from the BAM files including coverage for the regions of interest (ROI). Read coverage was calculated as the number of reads across the ROI, where the average Q-score for bases in the read exceeded Q20. Samples with at least 95% of amplicons having ≥300 reads with an average quality score of Q20 or better were considered successfully sequenced. For a sequence variant to be considered authentic, sequencing coverage of at least 300 Q20 reads or 10 variant reads and a reported variant frequency of at least 4% was required. Additional metrics recorded included whether the variation was within or adjacent to homopolymer regions, the number of bases at the variation, and the read strand bias. The metrics for the variations were integrated into a custom software application, CLS-Mutation-Review, which can also directly access the Integrated Genome Viewer (IGV) for focused visualization of the aligned reads and identified variation. Manual review of all the variations was performed using the CLS-Mutation Review application. Population variations and positions with known technical issues that had been identified from assay validation studies were tagged within the application to aid the review.

Amplicon Coverage. All 121 specimens were sequenced. On average, 98.6% (range 95-100%, 2.63 SD) of all amplicons met the minimum per-amplicon criteria of >300 reads, indicating adequate amplicon coverage for all 121 specimens.

Sanger Sequencing. Sanger sequencing assays were performed by standard methods. The specific genes and exons tested depended on the cancer type, but included EGFR, KRAS, HRAS, NRAS, BRAF, PIK3CA, TP53, and KIT.

Immunohistochemistry. Estrogen receptor (ER), progesterone receptor (PR), and HER-2 immunohistochemistry (IHC) testing was performed using standard assays. The stained slides were reviewed and classified as ER and PR positive when >1% of tumor cells showed positive nuclear staining. For the HER-2 assay, slides were classified as negative, equivocal, or positive based on CAP guidelines.

FISH. Commercial kits were used to assess HER-2 gene amplification (Vysis Path Vysion™ HER-2 DNA Probe Kit; Abbott Laboratories, Des Plaines, IL), ALK translocation (Vysis LSI ALK Break Apart Rearrangement Probe Kit; Abbott Laboratories), and ROS1 translocation (Cytocell ROS1 Breakapart kit; CytoCell, Compiegne, France).

### Example 4: Frequency and Breadth of Mutations Detected by the Solid Tumor Screening Assay of the Present Technology

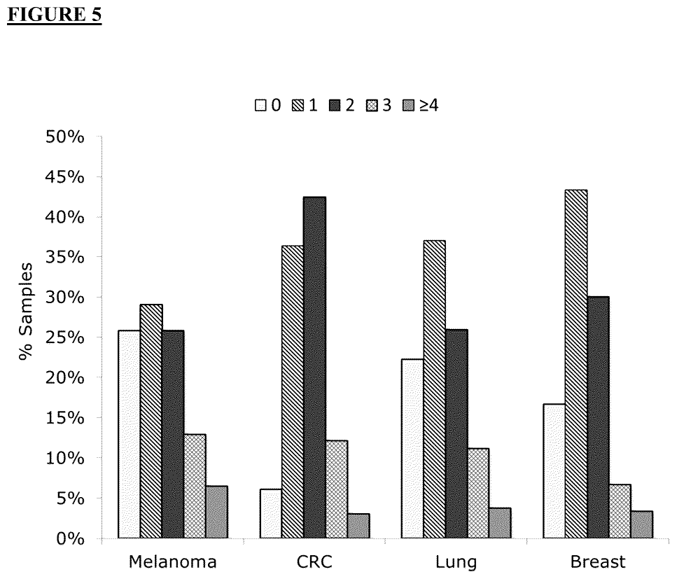

Results. Overall, 83% (100/121) of all FFPE specimens tested harbored at least one mutation, as determined by the solid tumor screening assay of the present technology: 74% (23/31) of melanoma samples; 94% (31/33) of colorectal cancer; 78% (21/27) of lung cancer; and 83% (25/30) in breast cancer (FIG. 5). A single gene mutation was observed in most tumor types i.e., melanoma, lung cancer, and breast cancer. Colorectal cancer was the exception, with a higher frequency of FFPE specimens harboring mutations in two or more genes. FFPE specimens harboring mutations in four or more genes were rarely observed in all tumor types.

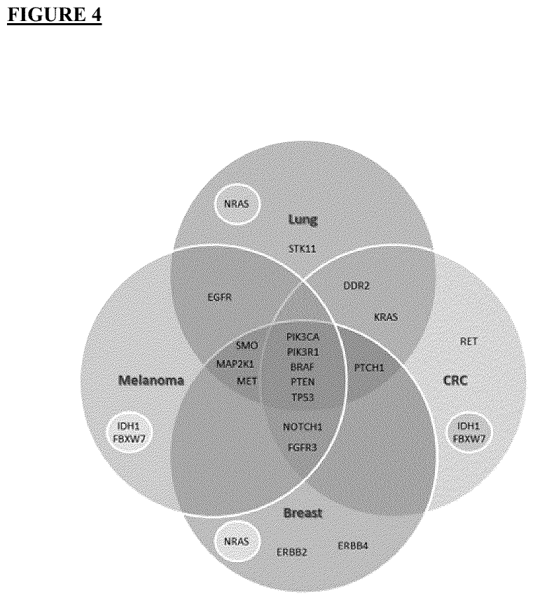

Mutations in 62% (21/34) of the genes assayed by the solid tumor screening panel of the present technology were detected. These observations support the selection of the plurality of amplicons that are interrogated in the solid tumor screening assay of the present technology. Of those 21 genes, BRAF, PIK3CA, PIK3R1, PTEN, and TP53 were mutated in all 4 tumor types; 11 genes were mutated in at least three tumor types; and 17 genes were mutated in at least two tumor types (FIG. 4). Moreover, 53% of all tumors (64/121) harbored mutations in TP53.

The only genes that were found to be mutated in a specific tumor type were STK11 in lung cancer; RET in colorectal cancer, and ERBB2, and ERBB4 in breast cancer. While the vast majority of specimens tested had only one gene mutation, at least 40% of each tumor type had mutations in >2 genes per specimen and 10% to 20% of each tumor type had mutations in ≥3 genes per specimen. Furthermore, six specimens harbored two different mutations within the same gene. These findings support the use of a single molecular profiling assay for multiple solid tumor types.

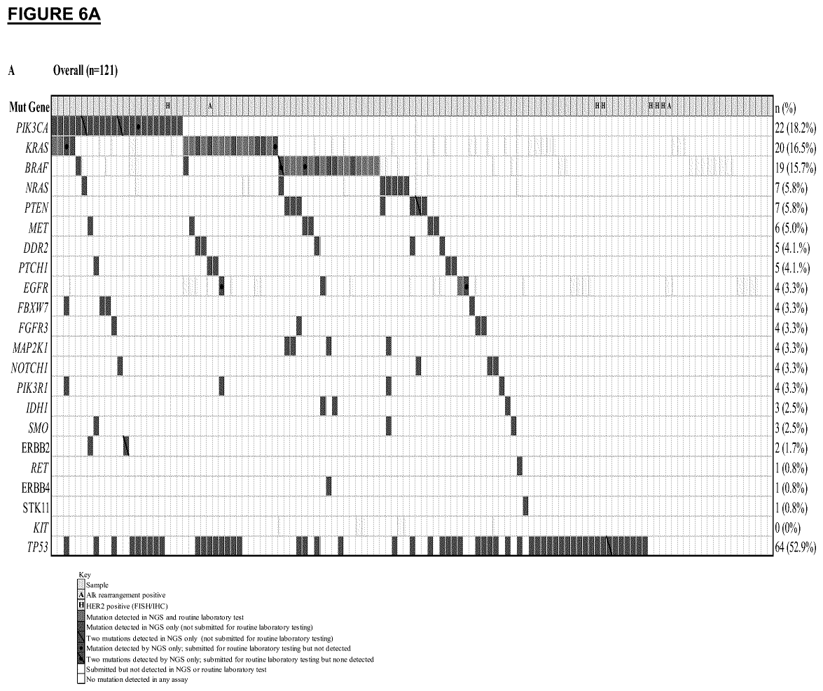

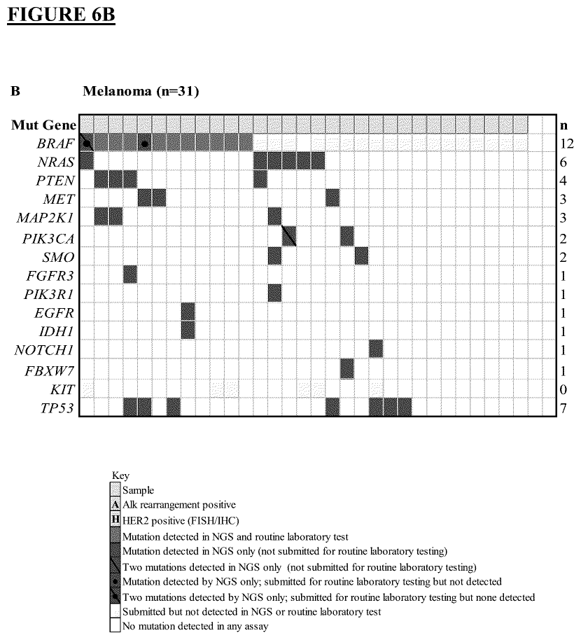

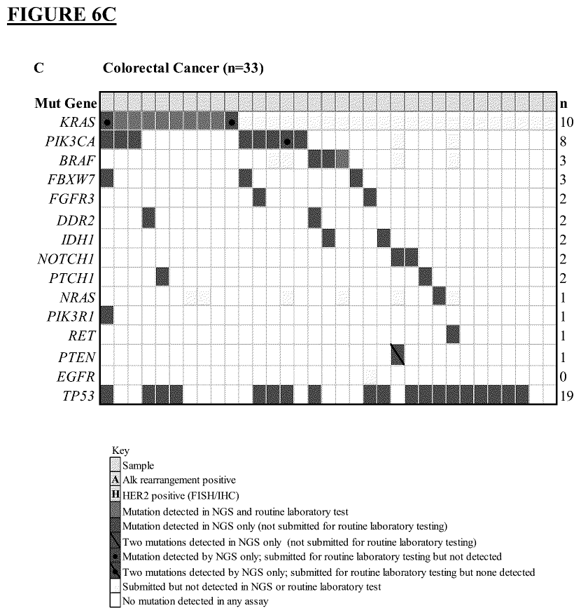

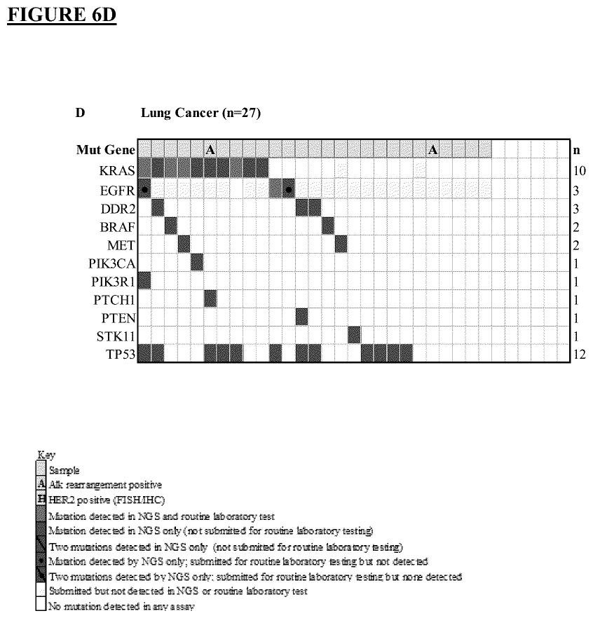

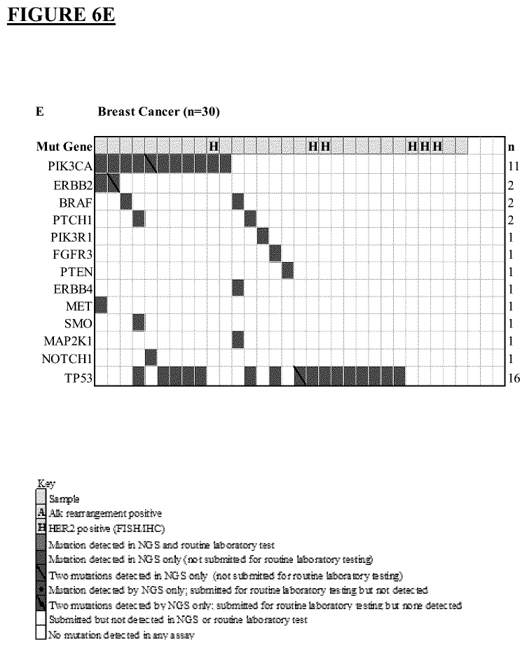

FIG. 6A shows the mutations detected by the solid tumor screening panel of the present technology for each individual specimen for all tumor types. As shown in FIG. 6B, BRAF was the most frequently mutated gene in melanoma samples (12/31; 39%) and included eight specimens with single-nucleotide substitutions (7 at V600E [GTG>GAG] and 1 at K483Q) and 5 with dinucleotide substitutions (V600E, GTG>GAA [n=4]; V600K, GTG>AAG [n=1]). Further, 19% (6/31) of the melanoma specimens had single-nucleotide NRAS substitutions (Q61K [n=2]; Q61R [n=3]; and G12R [n=1]), one of which also had two co-occurring BRAF mutations. Examples of additional mutated genes in the melanoma specimens included PTEN (13%), MET and MAP2K1 (10%), PIK3CA and SMO (6%), and single instances of FGFR3, PIK3R1, EGFR, IDH1, NOTCH1, and FBXW7 (3%). TP53 mutations were also found in 7 (23%) samples (FIG. 6B).

As shown in FIG. 6C, KRAS was the most frequently mutated gene in colorectal cancer samples (10/33 samples; 30%). All KRAS mutations in this group were single-nucleotide substitutions (G12V [n=2], G12D [n=2], G12C [n=1], G13D [n=3], L19F [n=1], and A146T [n=1]). Further, 24% (8/33) of colorectal cancer samples harbored single-nucleotide PIK3CA substitutions (1 each of G106R, R38H, E453K, H1044R, N1044K, E545K, Q546H, and C420R). BRAF mutations were detected in two (6%) of the 33 colorectal cancer samples (V600E [n=1] and G466V [n=1]). Examples of additional mutated genes in the colorectal cancer specimens included DDR2, IDH1, NOTCH1, PTCH1, NRAS, PIK3R1, RET, and PTEN. TP53 was also mutated in 19 (58%) of the samples (FIG. 6C).

As shown in FIG. 6D, 37% (10/27) of the lung cancer specimens harbored single-nucleotide KRAS substitutions. These included G12V (n=4), G12C (n=1), G13C (n=1), G12A (n=1), G12S (n=1), Q22K (n=1), and A146T (n=1). Further, 3 (11.1%) of the lung cancer specimens harbored EGFR mutations (AE746_A750 [n=2] and R680Q [n=1]) and 3 (11%) contained DDR2 mutations. Two (7%) of the 27 lung cancer samples had BRAF mutations (G464V and E501V). Some specimens with KRAS mutations had co-occurring mutations in at least one of EGFR, DDR2, BRAF, MET, PIK3CA, PIK3R1, PTCH1, and TP53 (FIG. 6D). A PTEN mutation was detected in one lung cancer specimen along with a co-occurring mutation in DDR2. Only 1 lung cancer specimen harbored an STK11 mutation. TP53 was also mutated in 12 (44%) of the lung cancer specimens (FIG. 6D).

As shown in FIG. 6E, ˜37% of the breast cancer specimens harbored PIK3CA mutations. One of the breast cancer samples had two co-occurring PIK3CA mutations (E542Q and H1047R) and a co-occurring mutation in NOTCH1. Two breast cancer specimens (7%) harbored ERBB2 mutations along with co-occurring PIK3CA mutations, one of which had two co-occurring ERBB2 point mutations (L755S and S310Y). Examples of additional mutated genes in breast cancer specimens included PTCH1 (7%) and single instances of PIK3R1, FGFR3, PTEN, ERBB4, MET, SMO, MAP2K1, and NOTCH1 (3%). TP53 was also mutated in 53% (16/30) of the breast cancer specimens (FIG. 6E).

These results demonstrate that the solid tumor screening assay of the present technology is useful in detecting a broad range of mutations in specifically targeted exons or gene regions of AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN in FFPE tissue samples. Therefore, DNA degradation and partial DNA denaturation during embedding of the tissues, long term storage of FFPE tissue samples and the presence of potential PCR inhibitors in tumor samples do not appear to influence the integrity of the solid tumor screening assay of the present technology.

Further, these results demonstrate that the solid tumor screening assay of the present technology is useful in methods for detecting at least one mutation in the plurality of cancer-related genes disclosed herein in a subject diagnosed with breast cancer, lung cancer, colorectal cancer, or melanoma.

### Example 5: The Solid Tumor Screening Assay of the Present Technology has Improved Sensitivity and Breadth of Coverage Compared to Sanger Sequencing

Single-Analyte Concordance of Mutation Detection Between the Solid Tumor Screening Assay of the Present Technology and Sanger sequencing. The Sanger sequencing tests ordered for each specimen are listed in Table 5.

TABLE 5
Sanger Sequencing Tests Initially
Ordered for the 121 FFPE Specimens

| Tumor type | Gene Tested | Number of case (%) | |
|---|---|---|---|
| Melanoma | BRAF | 30 | (96.8) |
| | KIT | 6 | (19.4) |
| | Total | 31 | |
| Lung cancer | EGFR | 26 | (96.3) |
| | KRAS | 11 | (40.7) |
| | ALK | 16 | (59.3) |
| | ROS1 | 1 | (3.7) |
| | Total | 27 | |
| Colorectal cancer | KRAS | 33 | (100) |
| | NRAS | 6 | (18.2) |
| | BRAF | 5 | (15.2) |
| | PIK3CA | 2 | (6.1) |
| | Total | 33 | |
| Breast cancer | ER | 30 | (100) |
| | PR | 30 | (100) |
| | HER-2 | 18 | (60.0) |

| | |
|---|---|
| Total | 30 |

The results of the initially ordered Sanger sequencing tests were compared with the solid tumor screening assay of the present technology for lung cancer, colorectal cancer, and melanoma samples (91 total FFPE specimens). Breast cancer specimens were not included in this analysis because no Sanger sequencing tests are routinely offered for breast cancer.

Some FFPE specimens had multiple tests ordered, yielding 121 Sanger sequencing assay results. 24 of the 121 Sanger sequencing assays were mutation positive (20%) and 97 were negative (80%). In contrast, the solid tumor screening assay of the present technology detected 34 mutations in the 91 FFPE specimens (28%), i.e., 10 more positive results compared to that observed with the initially ordered Sanger sequencing assays. Further, the solid tumor screening assay of the present technology detected every mutation identified by Sanger sequencing.

Discrepancies between the Sanger sequencing tests and the solid tumor screening assay of the present technology were discovered for all 3 tumor types. See Table 6.

TABLE 6
Discrepancies Between the Sanger Sequencing Tests and the
Solid Tumor Screening Assay of the Present Technology

| | Sample | | | |
|---|---|---|---|---|
| Tumor type | Number | Gene | Mutation | Allele Frequency (%) |
| Melanoma | 1 | BRAF | V600E^{a} | 56.1 |
| | 2 | BRAF | V600E^{a} | 59.2 |
| | 3 | BRAF | K483Q^{b} | 61.3 |
| Lung Cancer | 1 | EGFR | ΔE746-A750 | 6 |
| | 2 | EGFR | R680Q | 10.1 |
| | 3 | KRAS | A146T^{b} | 53.6 |
| | 4 | KRAS | R68M^{b} | 19.9 |
| Colorectal | 1 | KRAS | A146T^{b} | 34.1 |
| Cancer | 2 | KRAS | L19F^{b} | 15.7 |
| | 3 | PIK3CA | C420R^{b} | 15.4 |

^{a}dinucleotide mutation, not covered by cobas test
^{b}not covered (routinely sequenced) by Sanger sequencing test

Three FFPE melanoma samples were negative for BRAF mutations according to the initial cobas BRAF test and positive according to the solid tumor screening NGS assay of the present technology (Table 6). Two of the three mutations were BRAF V600E dinucleotide mutations (GTG to GAA) and the remaining one was a BRAF K483Q mutation.

Among the three discrepant colorectal cancer specimens, two had non-codon Dec. 13, 1961 KRAS mutations and one had an exon 7 PIK3CA mutation; none of these mutations were covered in the initial Sanger sequencing assays. See Table 6.

Of the four discrepant lung cancer specimens, two had EGFR mutations and two had non-codon Dec. 13, 1961 KRAS mutations; the KRAS mutations were not covered in the initial laboratory Sanger sequencing assays. See Table 6. Further, detection of the EGFR exon 19 deletion (AE746-A750) demonstrates that the solid tumor screening NGS assay of the present technology can detect mutations at a lower allelic fraction than Sanger sequencing, and is thus more sensitive than Sanger sequencing . . .

An analysis of the results indicates that the solid tumor screening NGS assay of the present technology has improved breadth of coverage and sensitivity over Sanger sequencing methods.

### Example 6: Additional Genes not Tested in the Initial Sanger Sequencing Assays

Melanoma: As shown in FIG. 6B, the solid tumor screening NGS assay of the present technology detected an additional 22 non-BRAF mutations in 12 of the 20 initially BRAF negative samples (60%)—FBXW7 (1), MAP2K1 (1), MET (2), NRAS (6), NOTCH1 (1), PIK3CA (2), PIK3R1 (1), SMO (2), PTEN (1) and TP53 (5). Moreover, seven of the 10 initially BRAF positive specimens harbored non-BRAF mutations-EGFR (1), FGFR3(1), IDH1(1), MAP2K1(2), MET (1), PTEN (3), and TP53 (2).

Colorectal Cancer. As shown in FIG. 6C, the solid tumor screening NGS assay of the present technology detected mutations in 22 (91%) of the 24 specimens that were either not submitted for or tested negative according to the initial Sanger sequencing assays. These included mutations in non-codon Dec. 13, 1961 KRAS (2), BRAF (3), PIK3CA (5), NRAS (1), RET (1), PTEN (1), and FGFR3 (1). Of the 8 specimens that tested positive for KRAS according to the initial Sanger sequencing tests, two harbored co-occurring PIK3CA mutations and one harbored a co-occurring DDR2 mutation (FIG. 6C).

Lung Cancer: As shown in FIG. 6D, the solid tumor screening NGS assay of the present technology detected at least one mutation in 10 (50%) of the 20 specimens that tested negative according to the initial Sanger sequencing assays. These included mutations in KRAS, BRAF, NRAS, DDR2, MET, and EGFR. Furthermore, four (57%) of the seven specimens that were positive according to the initial Sanger sequencing assay also harbored at least one additional mutation in EGFR, KRAS, BRAF, PIK3CA, or MET. Notably, an ALK rearrangement-positive specimen also harbored a codon 22 activating KRAS mutation (FIG. 6D).

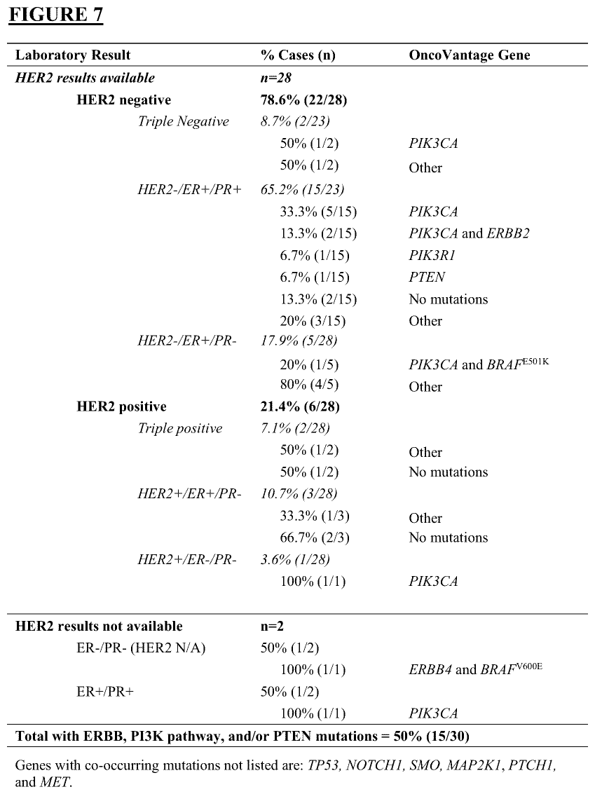

Breast cancer. The solid tumor screening NGS assay of the present technology was used to interrogate the 30 breast cancer specimens submitted for HER-2, ER and PR testing. See FIG. 6E. The HER-2 status of twenty-eight of the 30 breast cancer FFPE specimens was assayed by IHC and/or FISH. Of the 22 HER-2-negative specimens, 11 (50%) harbored mutations in RTK or P13K pathway genes: 9 mutations in PIK3CA; 2 in ERBB2; 1 in PIK3R1; 1 in PTEN; and 1 in BRAF (FIG. 7). Notably, all HER-2-negative samples harboring ERBB2 or BRAF mutations also had co-occurring PIK3CA mutations (FIG. 7). One of the six HER-2 positive specimens also harbored a PIK3CA mutation. The detailed mutation profile of the different combinations of HER-2, ER and PR status is shown in FIG. 7.

### Example 7: Implications on Treatment Selection

This Example demonstrates that the broader mutation profiling by the solid tumor screening NGS assay of the present technology provides additional information relevant to treatment selection beyond routine Sanger sequencing. This additional information was elucidated as a result of sequencing genes or gene regions not typically tested in a given tumor type, identifying co-occurring gene mutations, and the elevated sensitivity of the methods disclosed herein over standard Sanger sequencing.

As shown in FIGS. 6 (A)-(E) and FIG. 7, the solid tumor screening NGS assay of the present technology can provide a more complete molecular profile for a significant proportion of FFPE specimens submitted for molecular testing. For example, 76% (92/121) of the tested FFPE specimens yielded at least one additional gene mutation that was not identified by routine Sanger sequencing. Further, the methods of the present technology led to the identification of 16 actionable mutations (3 BRAF in melanoma, 2 KRAS, 2 BRAF and 1 NRAS in colorectal cancer, 2 EGFR and 6 KRAS in lung cancer) that were not detected by routine Sanger sequencing, which would most likely impact the chosen treatment strategy.

Moreover, the various co-occurring mutations identified by the methods of the present technology are useful in altering or designing a customized treatment regimen. These include consideration of alternative anti-HER-2 therapies (e.g., trastuzumab emtansine), EGFR TKIs (e.g., gefitinib), BRAF inhibitors (e.g., vemurifenab or dabrafenib), Src-family tyrosine kinase inhibitors (e.g., dasatinib), MEK inhibitors (e.g., selumetinib), and/or replacement or removal of classic anti-HER-2 therapies (e.g., trastuzumab), ALK/c-MET inhibitors (e.g., crizotinib), or anti-EGFR therapies (e.g., cetuximab).

Melanoma. The BRAF inhibitor vemurafenib is the most common targeted therapy for melanoma and is used treat BRAF V600 mutation-positive tumors. As shown in Table 5, the majority of melanoma specimens (30/31) were submitted for routine BRAF testing. 21 (68%) of the 31 melanoma samples tested negative according to the initial cobas BRAF test. However, two of the negative specimens were identified as harboring dual-nucleotide variants of BRAF V600E using the methods of the present technology, and are likely to benefit from treatment with vemurafenib.

Additionally, four BRAF mutation-negative specimens were identified as harboring mutations in NRAS (FIG. 6B). Clinical studies have shown that NRAS mutations may indicate a lack of responsiveness to vemurafenib. Su et al., N Engl J Med 366:207-215 (2012). However, these mutations may be responsive to MEK inhibitors. Ascierto et al., Lancet Oncol. 14(3):249-56 (2013).

Accordingly, the solid tumor screening NGS assay of the present technology is useful in determining whether a melanoma patient is likely to respond to treatment with vemurafenib or MEK inhibitors.

Also notable was the high proportion of BRAF-positive specimens with co-occurring mutations in EGFR, MET, FGFR3, IDH1, and/or PTEN. FIG. 6B. It is predicted that such co-occurring mutations will affect responses to targeted treatments. In addition, one PIK3CA and one MET mutation were detected in BRAF mutation-negative specimens. FIG. 6B.

Accordingly, the solid tumor screening NGS assay of the present technology is useful in determining whether a melanoma patient is likely to respond to treatment with a drug that modulates PIK3CA or MET activity.

Colorectal Cancer. Of the 33 colorectal cancer specimens initially tested by Sanger sequencing for KRAS, BRAF, NRAS, and/or PIK3CA mutations, 24 (73%) tested negative, thus suggesting a favorable response to anti-EGFR therapy. Strikingly, 11 (46%) of the specimens that tested negative according to Sanger sequencing were identified as positive for mutations in at least one of the EGFR downstream genes: non-codon Dec. 13, 1961 KRAS (2), BRAF (2), NRAS (1), PIK3CA (5), or PTEN (1). Thus, the solid tumor screening NGS assay of the present technology suggested a lack of responsiveness to anti-EGFR therapy in 61% of the colorectal cancer samples, in contrast to the 27% predicted by routine Sanger sequencing.

Accordingly, the solid tumor screening NGS assay of the present technology is useful in determining whether a colorectal cancer patient is likely to respond to treatment with anti-EGFR therapy (e.g., cetuximab).

Lung Cancer. In total, at least 37% (10/27) of the lung cancer FFPE specimens harbored mutations that suggested targeted treatment responses beyond what routine Sanger sequencing provided. As shown in FIG. 6D, initial Sanger sequencing of the 27 lung cancer FFPE specimens detected an EGFR mutation in one specimen, KRAS mutations in four, and an ALK rearrangement in two. The remaining 20 specimens were negative for all mutations initially tested. However, the solid tumor screening NGS assay of the present technology identified one specimen (that tested negative according to routine Sanger sequencing) as harboring an activating EGFR ΔE746_A750 mutation that had a low variant frequency of 6%.

Accordingly, the solid tumor screening NGS assay of the present technology is useful in determining whether a lung cancer patient is likely to respond to treatment with an EGFR TKI. In some embodiments, the patient has a low tumor cell burden.

Further, one lung cancer FFPE specimen tested positive for an ALK rearrangement according to routine Sanger sequencing, thereby suggesting a favorable response to crizotinib. However, the solid tumor screening NGS assay of the present technology identified an activating mutation in KRAS (codon 22), which has been associated with a lack of responsiveness to crizotinib.

Accordingly, the solid tumor screening NGS assay of the present technology is useful in determining whether a lung cancer patient is likely to respond to treatment with crizotinib.

Notably, the methods of the present technology identified an additional 9 (45%) out of the 20 lung cancer specimens that tested negative according to Sanger sequencing, as being positive for alterations in KRAS, BRAF, MET and DDR2. BRAF inhibitors vemurafenib and dabrafenib have been added to the NCCN Guidelines list of “Available Targeted Agents with Activity against Driver Event in Lung Cancer” for specimens harboring BRAF mutations. Sánchez-Torres et al., Transl Lung Cancer Res 2(3):244-250 (2013). Additionally, dasatinib is currently being studied for treatment of tumors harboring DDR2 mutations. Bail et al., Cancer Res. 2014 Oct. 27 (epub ahead of print).

Accordingly, the solid tumor screening NGS assay of the present technology is useful in determining whether a lung cancer patient is likely to respond to treatment with vemurafenib, dabrafenib, dasatinib or the MEK inhibitor selumetinib.

Breast Cancer. 28 of the 30 breast cancer specimens were submitted for HER-2, ER, and PR testing. Of these, six (21%) were HER-2 positive and therefore likely candidates for trastuzumab treatment (FIG. 7). Although five of the six HER-2-positive cases showed no additional mutations in ERBB or P13K pathway genes, the solid tumor screening NGS assay of the present technology detected a PIK3CA mutation in one sample. This is significant because PIK3CA mutations in HER-2 positive tumors have been associated with lack of responsiveness to conventional anti-HER-2 therapies (e.g. trastuzumab and lapatinib). Berns et al., Cancer Cell 12:395-402 (2007). However, trastuzumab emtansine (Kadcyla, Genentech) may be efficacious in treatment of such tumors, since concurrent PIK3CA mutations do not appear to alter outcome with this alternate therapy (American Association for Cancer Research [AACR] 104th Annual Meeting: Abstract LB-63. Presented Apr. 8, 2013).

Accordingly, the solid tumor screening NGS assay of the present technology is useful in determining whether a HER-2 positive patient diagnosed with breast cancer is likely to respond to treatment with anti-HER-2 therapies or trastuzumab emtansine.

Half (11/22) of the HER-2-negative breast cancer specimens harbored mutations in ERBB and/or P13K pathway genes. Mutations in PIK3CA were observed in the majority, including a subset that harbored co-occurring ERBB2 or BRAF mutations (FIG. 7). A mutation in PTEN was also observed. Overall observed frequencies of PIK3CA and PTEN mutations (37% and 3%, respectively) are consistent with TCGA data (doi: 10.1038/nature11412).

Importantly, breast cancers harboring PIK3CA kinase domain mutations or PTEN loss of function mutations (with no co-occurring BRAF or KRAS mutations) have been demonstrated to respond to treatment with P13K/AKT/mTOR pathway inhibitors such as everolimus. Sánchez-Torres et al., Transl Lung Cancer Res 2(3):244-250 (2013). It is anticipated that these mutations will serve as biomarkers for guiding therapeutic strategies involving anti-HER-2 therapies and P13K/AKT/mTOR pathway inhibitors.

Accordingly, the solid tumor screening NGS assay of the present technology is useful in determining whether a patient diagnosed with breast cancer is likely to respond to treatment with P13K/AKT/mTOR pathway inhibitors.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for predicting the likelihood of lack of responsiveness to treatment with an anti-HER-2 therapy in a HER-2 positive subject diagnosed as having breast cancer comprising:
(a) extracting genomic DNA from a formalin fixed paraffin-embedded specimen obtained from the HER-2 positive subject;
(b) generating a library comprising amplicons corresponding to each of a plurality of cancer-related genes, said plurality of cancer-related genes comprising AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN, wherein
(i) generating said library proceeds independently of using a bait set comprising nucleic acid sequences that are complementary to at least one of the plurality of amplicons, and
(ii) the quality of the genomic DNA extracted from the formalin fixed paraffin-embedded specimen is not assessed using quantitative PCR prior to generating the library;
(c) detecting at least one mutation in at least one of the plurality of amplicons; and
(d) identifying the HER-2 positive subject as having a likelihood of lack of responsiveness to treatment with an anti-HER-2 therapy, when a mutation in at least one of the amplicons corresponding to PIK3CA, PIK3R1 and PTEN is detected.

2. The method of claim 1, wherein the anti-HER-2 therapy is trastuzumab or lapatinib.

3. The method of claim 1, wherein the amplicons corresponding to PIK3CA are generated by a pair of primers selected from the group consisting of 5′ CCTAGTAGAATGTTTACTACCAA 3′ (SEQ ID NO.: 1) and 5′ CTGCTTCTTGAGTAACACTT 3′ (SEQ ID NO.: 2); 5′ CATGTTCATGCTGTGTATGT 3′ (SEQ ID NO.: 3) and 5′ GCTTCTTTACAAACGTTCAGAA 3′ (SEQ ID NO.: 4); 5′ TCTATGTTCGAACAGGTATCT 3′ (SEQ ID NO.: 5) and 5′ ACTGCTAAACACTAATATAACCTTTG 3′ (SEQ ID NO.: 6); 5′ TTGAAATGTGTTTTATAATTTAGACTAGT 3′ (SEQ ID NO.: 7) and 5′ CCATGAGGTACTGGCC 3′ (SEQ ID NO.: 8); 5′ TTGGTGTTACTGGATCAAATC 3′ (SEQ ID NO.: 9) and 5′ TGCTGAACCAGTCAAACT 3′ (SEQ ID NO.: 10); 5′ TATTATTTTATTTTACAGAGTAACAGACTAG 3′ (SEQ ID NO.: 11) and 5′ TTTAGCACTTACCTGTGACT 3′ (SEQ ID NO.: 12); 5′ TGGAATGCCAGAACTACA 3′ (SEQ ID NO.: 13) and 5′ GTGGAAGATCCAATCCATTTT 3′ (SEQ ID NO.: 14); 5′ GGAATGAATGGCTGAATTATG 3′ (SEQ ID NO.: 15) and 5′ GCGGTATAATCAGGAGTTTT 3′ (SEQ ID NO.: 16); 5′ AGTTGGCCTGAATCACTATA 3′ (SEQ ID NO.: 17) and 5′ GATGTTACTATTGTGACGATCTC 3′ (SEQ ID NO.: 18); 5′ GTAAGTGTTACTCAAGAAGC 3′ (SEQ ID NO.: 19) and 5′ ATAGGATATTGTATCATACCAATTTCT 3′ (SEQ ID NO.: 20); 5′ TCCACAGCTACACCATATAT 3′ (SEQ ID NO.: 21) and 5′ AGCATCAGCATTTGACTTTA 3′ (SEQ ID NO.: 22); 5′ TACACAGACACTCTAGTATCTG 3′ (SEQ ID NO.: 23) and 5′ GAAGGTTTGACTGCCATAAA 3′ (SEQ ID NO.: 24); 5′ ATGACAAAGAACAGCTCAAA 3′ (SEQ ID NO.: 25) and 5′ GAGATCAGCCAAATTCAGTT 3′ (SEQ ID NO.: 26); 5′ GATGTGTTACAAGGCTTATCTA 3′ (SEQ ID NO.: 27) and 5′ GCCTCTTGCTCAGTTTTATC 3′ (SEQ ID NO.: 28); 5′ GAGGCTTTGGAGTATTTCA 3′ (SEQ ID NO.: 29) and 5′ CTGCTGAGAGTTATTAACAGT 3′ (SEQ ID NO.: 30); and 5′ GCTTTTGGAGTCCTATTGT 3′ (SEQ ID NO.: 31) and 5′ CACAAACTAGAGTCACACAC 3′ (SEQ ID NO.: 32).

4. The method of claim 1, wherein the HER-2 positive subject is treated with trastuzumab emtansine, when a mutation in at least one of the amplicons corresponding to PIK3CA, PIK3R1 and PTEN is detected.

5. The method of claim 1, wherein the HER-2 positive status of the subject is assayed by immunohistochemistry (IHC) or fluorescent in situ hybridization (FISH).

6. The method of claim 1, further comprising ligating an adapter sequence to the ends of the amplicons generated in step (b).

7. The method of claim 1, wherein the library comprising amplicons corresponding to each of the plurality of cancer-related genes is generated using no more than 10 ng of extracted genomic DNA from the formalin fixed paraffin-embedded tumor sample.

8. The method of claim 1, wherein the library comprising amplicons corresponding to each of the plurality of cancer-related genes is generated using 11-25 ng of extracted genomic DNA from the formalin fixed paraffin-embedded tumor sample.

9. The method of claim 1, wherein the at least one mutation in at least one of the plurality of amplicons is detected using high throughput massive parallel sequencing.

10. The method of claim 9, wherein the high throughput massive parallel sequencing is performed using pyrosequencing, reversible dye-terminator sequencing, SOLID sequencing, Ion semiconductor sequencing, Helioscope single molecule sequencing, sequencing by synthesis, sequencing by ligation, or SMRT™ sequencing.

11. The method of claim 1, wherein the adapter sequence is a P5 adapter, P7 adapter, P1 adapter, A adapter, or Ion Xpress™ barcode adapter.

12. The method of claim 1, wherein the plurality of amplicons further comprise a unique index sequence.

13. The method of claim 1, wherein the formalin fixed paraffin-embedded tumor sample is a heterogeneous tumor.

14. The method of claim 13, wherein 5% of the cells of the heterogeneous tumor harbor at least one mutation in at least one of the plurality of amplicons.

15. A method for selecting a subject for treatment with a EGFR tyrosine kinase inhibitor and at least one additional agent comprising:
(a) extracting genomic DNA from a formalin fixed paraffin-embedded specimen obtained from the subject;
(b) generating a library comprising amplicons corresponding to each of a plurality of cancer-related genes, said plurality of cancer-related genes comprising AKT1, ERBB2, FOXL2, IDH2, NRAS, RET, ALK, ERBB4, GNA11, KIT, PDGFRA, SMO, BRAF, FBXW7, GNAQ, KRAS, PIK3CA, STK11, CTNNB1, FGFR2, GNAS, MAP2K1, PIK3R1, TP53, DDR2, FGFR3, HRAS, MET, PTCH1, EGFR, FGFR4, IDH1, NOTCH1, and PTEN, wherein
(i) generating said library proceeds independently of using a bait set comprising nucleic acid sequences that are complementary to at least one of the plurality of amplicons, and
(ii) the quality of the genomic DNA extracted from the formalin fixed paraffin-embedded specimen is not assessed using quantitative PCR prior to generating the library;
(c) detecting at least one mutation in at least one of the plurality of amplicons; and
(d) selecting the subject for treatment with a EGFR tyrosine kinase inhibitor and at least one additional agent, if a mutation in at least one of the amplicons corresponding to EGFR, and a mutation in at least one of the amplicons corresponding to KRAS, PIK3R1 and BRAF are detected.

16. The method of claim 15, wherein the formalin fixed paraffin-embedded specimen is a heterogeneous tumor.

17. The method of claim 16, wherein 5%-10% of the cells of the heterogeneous tumor harbor at least one mutation in at least one of the plurality of amplicons.

18. The method of claim 16, wherein at least 10% of the cells of the heterogeneous tumor harbor at least one mutation in at least one of the plurality of amplicons.

19. The method of claim 15, wherein the EGFR tyrosine kinase inhibitor is gefitinib or erlotinib.

20. The method of claim 15, wherein the at least one mutation in at least one of the plurality of amplicons is detected using high throughput massive parallel sequencing.


SEQUENCE LISTING (464 sequences)

SEQ ID NO: 1 - DNA, 23 bp, synthetic construct
  misc_feature 1..23: note = Description of Artificial Sequence: Synthetic primer
  source 1..23: mol_type = other DNA
cctagtagaatgtttactaccaa

SEQ ID NO: 2 - DNA, 20 bp, synthetic construct
  misc_feature 1..20: note = Description of Artificial Sequence: Synthetic primer
  source 1..20: mol_type = other DNA
ctgcttcttgagtaacactt

SEQ ID NO: 3 - DNA, 20 bp, synthetic construct
  misc_feature 1..20: note = Description of Artificial Sequence: Synthetic primer
  source 1..20: mol_type = other DNA
catgttcatgctgtgtatgt

SEQ ID NO: 4 - DNA, 22 bp, synthetic construct
  misc_feature 1..22: note = Description of Artificial Sequence: Synthetic primer
  source 1..22: mol_type = other DNA
gcttctttacaaacgttcagaa

SEQ ID NO: 5 - DNA, 21 bp, synthetic construct
  misc_feature 1..21: note = Description of Artificial Sequence: Synthetic primer
  source 1..21: mol_type = other DNA
tctatgttcgaacaggtatct

SEQ ID NO: 6 - DNA, 26 bp, synthetic construct
  misc_feature 1..26: note = Description of Artificial Sequence: Synthetic primer
  source 1..26: mol_type = other DNA
actgctaaacactaatataacctttg

SEQ ID NO: 7 - DNA, 29 bp, synthetic construct
  misc_feature 1..29: note = Description of Artificial Sequence: Synthetic primer
  source 1..29: mol_type = other DNA
ttgaaatgtgttttataatttagactagt

SEQ ID NO: 8 - DNA, 16 bp, synthetic construct
  misc_feature 1..16: note = Description of Artificial Sequence: Synthetic primer
  source 1..16: mol_type = other DNA
ccatgaggtactggcc

SEQ ID NO: 9 - DNA, 21 bp, synthetic construct
  misc_feature 1..21: note = Description of Artificial Sequence: Synthetic primer
  source 1..21: mol_type = other DNA
ttggtgttactggatcaaatc

SEQ ID NO: 10 - DNA, 18 bp, synthetic construct
  misc_feature 1..18: note = Description of Artificial Sequence: Synthetic primer
  source 1..18: mol_type = other DNA
tgctgaaccagtcaaact

SEQ ID NO: 11 - DNA, 31 bp, synthetic construct
  misc_feature 1..31: note = Description of Artificial Sequence: Synthetic primer
  source 1..31: mol_type = other DNA
tattattttattttacagagtaacagactag

SEQ ID NO: 12 - DNA, 20 bp, synthetic construct
  misc_feature 1..20: note = Description of Artificial Sequence: Synthetic primer
  source 1..20: mol_type = other DNA
tttagcacttacctgtgact

SEQ ID NO: 13 - DNA, 18 bp, synthetic construct
  misc_feature 1..18: note = Description of Artificial Sequence: Synthetic primer
  source 1..18: mol_type = other DNA
tggaatgccagaactaca

SEQ ID NO: 14 - DNA, 21 bp, synthetic construct
  misc_feature 1..21: note = Description of Artificial Sequence: Synthetic primer
  source 1..21: mol_type = other DNA
gtggaagatccaatccatttt

SEQ ID NO: 15 - DNA, 21 bp, synthetic construct
  misc_feature 1..21: note = Description of Artificial Sequence: Synthetic primer
  source 1..21: mol_type = other DNA
ggaatgaatggctgaattatg

SEQ ID NO: 16 - DNA, 20 bp, synthetic construct
  misc_feature 1..20: note = Description of Artificial Sequence: Synthetic primer
  source 1..20: mol_type = other DNA
gcggtataatcaggagtttt

SEQ ID NO: 17 - DNA, 20 bp, synthetic construct
  misc_feature 1..20: note = Description of Artificial Sequence: Synthetic primer
  source 1..20: mol_type = other DNA
agttggcctgaatcactata

SEQ ID NO: 18 - DNA, 23 bp, synthetic construct
  misc_feature 1..23: note = Description of Artificial Sequence: Synthetic primer
  source 1..23: mol_type = other DNA
gatgttactattgtgacgatctc

SEQ ID NO: 19 - DNA, 20 bp, synthetic construct
  misc_feature 1..20: note = Description of Artificial Sequence: Synthetic primer
  source 1..20: mol_type = other DNA
gtaagtgttactcaagaagc

SEQ ID NO: 20 - DNA, 27 bp, synthetic construct
  misc_feature 1..27: note = Description of Artificial Sequence: Synthetic primer
  source 1..27: mol_type = other DNA
ataggatattgtatcataccaatttct

SEQ ID NO: 21 - DNA, 20 bp, synthetic construct
  misc_feature 1..20: note = Description of Artificial Sequence: Synthetic primer
  source 1..20: mol_type = other DNA
tccacagctacaccatatat

SEQ ID NO: 22 - DNA, 20 bp, synthetic construct
  misc_feature 1..20: note = Description of Artificial Sequence: Synthetic primer
  source 1..20: mol_type = other DNA
agcatcagcatttgacttta

SEQ ID NO: 23 - DNA, 22 bp, synthetic construct
  misc_feature 1..22: note = Description of Artificial Sequence: Synthetic primer
  source 1..22: mol_type = other DNA
tacacagacactctagtatctg

SEQ ID NO: 24 - DNA, 20 bp, synthetic construct
  misc_feature 1..20: note = Description of Artificial Sequence: Synthetic primer
  source 1..20: mol_type = other DNA
gaaggtttgactgccataaa

SEQ ID NO: 25 - DNA, 20 bp, synthetic construct
  misc_feature 1..20: note = Description of Artificial Sequence: Synthetic primer
  source 1..20: mol_type = other DNA
atgacaaagaacagctcaaa

SEQ ID NO: 26 - DNA, 20 bp, synthetic construct
  misc_feature 1..20: note = Description of Artificial Sequence: Synthetic primer
  source 1..20: mol_type = other DNA
gagatcagccaaattcagtt

SEQ ID NO: 27 - DNA, 22 bp, synthetic construct
  misc_feature 1..22: note = Description of Artificial Sequence: Synthetic primer
  source 1..22: mol_type = other DNA
gatgtgttacaaggcttatcta

SEQ ID NO: 28 - DNA, 20 bp, synthetic construct
  misc_feature 1..20: note = Description of Artificial Sequence: Synthetic primer
  source 1..20: mol_type = other DNA
gcctcttgctcagttttatc

SEQ ID NO: 29 - DNA, 19 bp, synthetic construct
  misc_feature 1..19: note = Description of Artificial Sequence: Synthetic primer
  source 1..19: mol_type = other DNA
gaggctttggagtatttca

SEQ ID NO: 30 - DNA, 21 bp, synthetic construct
  misc_feature 1..21: note = Description of Artificial Sequence: Synthetic primer
  source 1..21: mol_type = other DNA
ctgctgagagttattaacagt

SEQ ID NO: 31 - DNA, 19 bp, synthetic construct
  misc_feature 1..19: note = Description of Artificial Sequence: Synthetic primer
  source 1..19: mol_type = other DNA
gcttttggagtcctattgt

SEQ ID NO: 32 - DNA, 20 bp, synthetic construct
  misc_feature 1..20: note = Description of Artificial Sequence: Synthetic primer
  source 1..20: mol_type = other DNA
cacaaactagagtcacacac

SEQ ID NO: 33 - DNA, 19 bp, synthetic construct
  misc_feature 1..19: note = Description of Artificial Sequence: Synthetic primer
  source 1..19: mol_type = other DNA
gggttttgggctgatatta

SEQ ID NO: 34 - DNA, 20 bp, synthetic construct
  misc_feature 1..20: note = Description of Artificial Sequence: Synthetic primer
  source 1..20: mol_type = other DNA
ccacagaactgaaggttaat

SEQ ID NO: 35 - DNA, 31 bp, synthetic construct
  misc_feature 1..31: note = Description of Artificial Sequence: Synthetic primer
  source 1..31: mol_type = other DNA
ttatccattgaatttattttaatctttctag

SEQ ID NO: 36 - DNA, 18 bp, synthetic construct
  misc_feature 1..18: note = Description of Artificial Sequence: Synthetic primer
  source 1..18: mol_type = other DNA
gggatgtgcgggtatatt

SEQ ID NO: 37 - DNA, 20 bp, synthetic construct
  misc_feature 1..20: note = Description of Artificial Sequence: Synthetic primer
  source 1..20: mol_type = other DNA
gtcttgcagtaagagattgt

SEQ ID NO: 38 - DNA, 16 bp, synthetic construct
  misc_feature 1..16: note = Description of Artificial Sequence: Synthetic primer
  source 1..16: mol_type = other DNA
tctttgctgtaccgct

SEQ ID NO: 39 - DNA, 16 bp, synthetic construct
  misc_feature 1..16: note = Description of Artificial Sequence: Synthetic primer
  source 1..16: mol_type = other DNA
gtttcttttgcctgca

SEQ ID NO: 40 - DNA, 21 bp, synthetic construct
  misc_feature 1..21: note = Description of Artificial Sequence: Synthetic primer
  source 1..21: mol_type = other DNA
tggataaggtctggtttaatg

SEQ ID NO: 41 - DNA, 21 bp, synthetic construct
  misc_feature 1..21: note = Description of Artificial Sequence: Synthetic primer
  source 1..21: mol_type = other DNA
gctacaattcaggatgagtta

SEQ ID NO: 42 - DNA, 21 bp, synthetic construct
  misc_feature 1..21: note = Description of Artificial Sequence: Synthetic primer
  source 1..21: mol_type = other DNA
tcttctgctatcaccatcttt

SEQ ID NO: 43 - DNA, 20 bp, synthetic construct
  misc_feature 1..20: note = Description of Artificial Sequence: Synthetic primer
  source 1..20: mol_type = other DNA
ccatcatgatgagaagacat

SEQ ID NO: 44 - DNA, 20 bp, synthetic construct
  misc_feature 1..20: note = Description of Artificial Sequence: Synthetic primer
  source 1..20: mol_type = other DNA
ttgctggagatacatacact

SEQ ID NO: 45 - DNA, 20 bp, synthetic construct
  misc_feature 1..20: note = Description of Artificial Sequence: Synthetic primer
  source 1..20: mol_type = other DNA
gtggtcactaaaccttaaga

SEQ ID NO: 46 - DNA, 20 bp, synthetic construct
  misc_feature 1..20: note = Description of Artificial Sequence: Synthetic primer
  source 1..20: mol_type = other DNA
ggcttaccttagtgtaagag

SEQ ID NO: 47 - DNA, 22 bp, synthetic construct
  misc_feature 1..22: note = Description of Artificial Sequence: Synthetic primer
  source 1..22: mol_type = other DNA
tttcatcgagatgggaaatatg

SEQ ID NO: 48 - DNA, 21 bp, synthetic construct
  misc_feature 1..21: note = Description of Artificial Sequence: Synthetic primer
  source 1..21: mol_type = other DNA
acctgttggtatttggatact

SEQ ID NO: 49 - DNA, 24 bp, synthetic construct
  misc_feature 1..24: note = Description of Artificial Sequence: Synthetic primer
  source 1..24: mol_type = other DNA
agaagataatattgaagctgtagg

SEQ ID NO: 50 - DNA, 29 bp, synthetic construct
  misc_feature 1..29: note = Description of Artificial Sequence: Synthetic primer
  source 1..29: mol_type = other DNA
agaactcttattttttaatctgattttca

SEQ ID NO: 51 - DNA, 21 bp, synthetic construct
  misc_feature 1..21: note = Description of Artificial Sequence: Synthetic primer
  source 1..21: mol_type = other DNA
ggacagctattgaagcattta

SEQ ID NO: 52 - DNA, 20 bp, synthetic construct
  misc_feature 1..20: note = Description of Artificial Sequence: Synthetic primer
  source 1..20: mol_type = other DNA
cacaagaacaagggaaacac

SEQ ID NO: 53 - DNA, 17 bp, synthetic construct
  misc_feature 1..17: note = Description of Artificial Sequence: Synthetic primer
  source 1..17: mol_type = other DNA
gcaggcagctgagtatc

SEQ ID NO: 54 - DNA, 22 bp, synthetic construct
  misc_feature 1..22: note = Description of Artificial Sequence: Synthetic primer
  source 1..22: mol_type = other DNA
tcatcctgaattgtagcaatca

SEQ ID NO: 55 - DNA, 17 bp, synthetic construct
  misc_feature 1..17: note = Description of Artificial Sequence: Synthetic primer
  source 1..17: mol_type = other DNA
cagcttctgccatctct

SEQ ID NO: 56 - DNA, 15 bp, synthetic construct
  misc_feature 1..15: note = Description of Artificial Sequence: Synthetic primer
  source 1..15: mol_type = other DNA
agcagccgcagaaat

SEQ ID NO: 57 - DNA, 19 bp, synthetic construct
  misc_feature 1..19: note = Description of Artificial Sequence: Synthetic primer
  source 1..19: mol_type = other DNA
gtggctttttgtttgtttg

SEQ ID NO: 58 - DNA, 21 bp, synthetic construct
  misc_feature 1..21: note = Description of Artificial Sequence: Synthetic primer
  source 1..21: mol_type = other DNA
cactctaacaagcagataact

SEQ ID NO: 59 - DNA, 29 bp, synthetic construct
  misc_feature 1..29: note = Description of Artificial Sequence: Synthetic primer
  source 1..29: mol_type = other DNA
tacttgttaattaaaaattcaagagtttt

SEQ ID NO: 60 - DNA, 20 bp, synthetic construct
  misc_feature 1..20: note = Description of Artificial Sequence: Synthetic primer
  source 1..20: mol_type = other DNA
cttagccattggtcaagatc

SEQ ID NO: 61 - DNA, 17 bp, synthetic construct
  misc_feature 1..17: note = Description of Artificial Sequence: Synthetic primer
  source 1..17: mol_type = other DNA
acaatcatgttgcagca

SEQ ID NO: 62 - DNA, 28 bp, synthetic construct
  misc_feature 1..28: note = Description of Artificial Sequence: Synthetic primer
  source 1..28: mol_type = other DNA
aaaaacatcaaaaaataacttacctttt

SEQ ID NO: 63 - DNA, 19 bp, synthetic construct
  misc_feature 1..19: note = Description of Artificial Sequence: Synthetic primer
  source 1..19: mol_type = other DNA
agaggcgctatgtgtatta

SEQ ID NO: 64 - DNA, 21 bp, synthetic construct
  misc_feature 1..21: note = Description of Artificial Sequence: Synthetic primer
  source 1..21: mol_type = other DNA
catggaaggatgagaatttca

SEQ ID NO: 65 - DNA, 20 bp, synthetic construct
  misc_feature 1..20: note = Description of Artificial Sequence: Synthetic primer
  source 1..20: mol_type = other DNA
ggaagacaagttcatgtact

SEQ ID NO: 66 - DNA, 24 bp, synthetic construct
  misc_feature 1..24: note = Description of Artificial Sequence: Synthetic primer
  source 1..24: mol_type = other DNA
ctgtccttattttggatatttctc

SEQ ID NO: 67 - DNA, 32 bp, synthetic construct
  misc_feature 1..32: note = Description of Artificial Sequence: Synthetic primer
  source 1..32: mol_type = other DNA
attaattaaatatgtcatttcatttctttttc

SEQ ID NO: 68 - DNA, 21 bp, synthetic construct
  misc_feature 1..21: note = Description of Artificial Sequence: Synthetic primer
  source 1..21: mol_type = other DNA
gctatcgatttcttgatcaca

SEQ ID NO: 69 - DNA, 22 bp, synthetic construct
  misc_feature 1..22: note = Description of Artificial Sequence: Synthetic primer
  source 1..22: mol_type = other DNA
tgagtcatatttgtgggttttc

SEQ ID NO: 70 - DNA, 22 bp, synthetic construct
  misc_feature 1..22: note = Description of Artificial Sequence: Synthetic primer
  source 1..22: mol_type = other DNA
tgatcaggttcattgtcactaa

SEQ ID NO: 71 - DNA, 21 bp, synthetic construct
  misc_feature 1..21: note = Description of Artificial Sequence: Synthetic primer
  source 1..21: mol_type = other DNA
tttgattgctgcatatttcag

SEQ ID NO: 72 - DNA, 23 bp, synthetic construct
  misc_feature 1..23: note = Description of Artificial Sequence: Synthetic primer
  source 1..23: mol_type = other DNA
tcaaagcattcttaccttactac

SEQ ID NO: 73 - DNA, 25 bp, synthetic construct
  misc_feature 1..25: note = Description of Artificial Sequence: Synthetic primer
  source 1..25: mol_type = other DNA
ttttaaacttttcttttagttgtgc

SEQ ID NO: 74 - DNA, 20 bp, synthetic construct
  misc_feature 1..20: note = Description of Artificial Sequence: Synthetic primer
  source 1..20: mol_type = other DNA
actcgataatctggatgact

SEQ ID NO: 75 - DNA, 28 bp, synthetic construct
  misc_feature 1..28: note = Description of Artificial Sequence: Synthetic primer
  source 1..28: mol_type = other DNA
caatttagtgaaataactataatggaac

SEQ ID NO: 76 - DNA, 19 bp, synthetic construct
  misc_feature 1..19: note = Description of Artificial Sequence: Synthetic primer
  source 1..19: mol_type = other DNA
agtgccactggtctataat

SEQ ID NO: 77 - DNA, 23 bp, synthetic construct
  misc_feature 1..23: note = Description of Artificial Sequence: Synthetic primer
  source 1..23: mol_type = other DNA
cctgtgaaataatactggtatgt

SEQ ID NO: 78 - DNA, 22 bp, synthetic construct
  misc_feature 1..22: note = Description of Artificial Sequence: Synthetic primer
  source 1..22: mol_type = other DNA
ctactttgatatcaccacacac

SEQ ID NO: 79 - DNA, 19 bp, synthetic construct
  misc_feature 1..19: note = Description of Artificial Sequence: Synthetic primer
  source 1..19: mol_type = other DNA
tagagcgtgcagataatga

SEQ ID NO: 80 - DNA, 17 bp, synthetic construct
  misc_feature 1..17: note = Description of Artificial Sequence: Synthetic primer
  source 1..17: mol_type = other DNA
tcaacaacccccacaaa

SEQ ID NO: 81 - DNA, 21 bp, synthetic construct
  misc_feature 1..21: note = Description of Artificial Sequence: Synthetic primer
  source 1..21: mol_type = other DNA
ctttctctaggtgaagctgta

SEQ ID NO: 82 - DNA, 20 bp, synthetic construct
  misc_feature 1..20: note = Description of Artificial Sequence: Synthetic primer
  source 1..20: mol_type = other DNA
ggttcattctctggatcaga

SEQ ID NO: 83 - DNA, 18 bp, synthetic construct
  misc_feature 1..18: note = Description of Artificial Sequence: Synthetic primer
  source 1..18: mol_type = other DNA
ctgaggtggaagagacag

SEQ ID NO: 84 - DNA, 17 bp, synthetic construct
  misc_feature 1..17: note = Description of Artificial Sequence: Synthetic primer
  source 1..17: mol_type = other DNA
actggttctcactcacc

SEQ ID NO: 85 - DNA, 16 bp, synthetic construct
  misc_feature 1..16: note = Description of Artificial Sequence: Synthetic primer
  source 1..16: mol_type = other DNA
gccagcaaagcagtag

SEQ ID NO: 86 - DNA, 21 bp, synthetic construct
  misc_feature 1..21: note = Description of Artificial Sequence: Synthetic primer
  source 1..21: mol_type = other DNA
tggaaaaatagcctcaattct

SEQ ID NO: 87 - DNA, 22 bp, synthetic construct
  misc_feature 1..22: note = Description of Artificial Sequence: Synthetic primer
  source 1..22: mol_type = other DNA
caccacattacatacttaccat

SEQ ID NO: 88 - DNA, 22 bp, synthetic construct
  misc_feature 1..22: note = Description of Artificial Sequence: Synthetic primer
  source 1..22: mol_type = other DNA
ctctggaaaagagtaattcaca

SEQ ID NO: 89 - DNA, 23 bp, synthetic construct
  misc_feature 1..23: note = Description of Artificial Sequence: Synthetic primer
  source 1..23: mol_type = other DNA
cgtatttatagctgatttgatgg

SEQ ID NO: 90 - DNA, 18 bp, synthetic construct
  misc_feature 1..18: note = Description of Artificial Sequence: Synthetic primer
  source 1..18: mol_type = other DNA
ggacttcacctgacagat

SEQ ID NO: 91 - DNA, 21 bp, synthetic construct
  misc_feature 1..21: note = Description of Artificial Sequence: Synthetic primer
  source 1..21: mol_type = other DNA
ctttggtttctcttggtctag

SEQ ID NO: 92 - DNA, 20 bp, synthetic construct
  misc_feature 1..20: note = Description of Artificial Sequence: Synthetic primer
  source 1..20: mol_type = other DNA
gagtttctgcagattgactt

SEQ ID NO: 93 - DNA, 18 bp, synthetic construct
  misc_feature 1..18: note = Description of Artificial Sequence: Synthetic primer
  source 1..18: mol_type = other DNA
gtgtttcctttgcagatg

SEQ ID NO: 94 - DNA, 19 bp, synthetic construct
  misc_feature 1..19: note = Description of Artificial Sequence: Synthetic primer
  source 1..19: mol_type = other DNA
tcacatgcctctttctcta

SEQ ID NO: 95 - DNA, 16 bp, synthetic construct
  misc_feature 1..16: note = Description of Artificial Sequence: Synthetic primer
  source 1..16: mol_type = other DNA
cctggctctgactcac

SEQ ID NO: 96 - DNA, 17 bp, synthetic construct
  misc_feature 1..17: note = Description of Artificial Sequence: Synthetic primer
  source 1..17: mol_type = other DNA
tttccctgccaagtgat

SEQ ID NO: 97 - DNA, 14 bp, synthetic construct
  misc_feature 1..14: note = Description of Artificial Sequence: Synthetic primer
  source 1..14: mol_type = other DNA
tgcagcggtgttgt

SEQ ID NO: 98 - DNA, 15 bp, synthetic construct
  misc_feature 1..15: note = Description of Artificial Sequence: Synthetic primer
  source 1..15: mol_type = other DNA
tctcggctcaaggac

SEQ ID NO: 99 - DNA, 20 bp, synthetic construct
  misc_feature 1..20: note = Description of Artificial Sequence: Synthetic primer
  source 1..20: mol_type = other DNA
ttcctcagttacaccaatct

SEQ ID NO: 100 - DNA, 17 bp, synthetic construct
  misc_feature 1..17: note = Description of Artificial Sequence: Synthetic primer
  source 1..17: mol_type = other DNA
gcctttggggttacttt

SEQ ID NO: 101 - DNA, 18 bp, synthetic construct
  misc_feature 1..18: note = Description of Artificial Sequence: Synthetic primer
  source 1..18: mol_type = other DNA
gctgctggagaagagata

SEQ ID NO: 102 - DNA, 17 bp, synthetic construct
  misc_feature 1..17: note = Description of Artificial Sequence: Synthetic primer
  source 1..17: mol_type = other DNA
aggtggctggttatgtc

SEQ ID NO: 103 - DNA, 21 bp, synthetic construct
  misc_feature 1..21: note = Description of Artificial Sequence: Synthetic primer
  source 1..21: mol_type = other DNA
ggagataagtgatggagatgt

SEQ ID NO: 104 - DNA, 16 bp, synthetic construct
  misc_feature 1..16: note = Description of Artificial Sequence: Synthetic primer
  source 1..16: mol_type = other DNA
ggatcggcctcttcat

SEQ ID NO: 105 - DNA, 17 bp, synthetic construct
  misc_feature 1..17: note = Description of Artificial Sequence: Synthetic primer
  source 1..17: mol_type = other DNA
cccaaccaagctctctt

SEQ ID NO: 106 - DNA, 15 bp, synthetic construct
  misc_feature 1..15: note = Description of Artificial Sequence: Synthetic primer
  source 1..15: mol_type = other DNA
actgacgtgcctctc

SEQ ID NO: 107 - DNA, 17 bp, synthetic construct
  misc_feature 1..17: note = Description of Artificial Sequence: Synthetic primer
  source 1..17: mol_type = other DNA
gcagggtcttctctgtt

SEQ ID NO: 108 - DNA, 21 bp, synthetic construct
  misc_feature 1..21: note = Description of Artificial Sequence: Synthetic primer
  source 1..21: mol_type = other DNA
ccaacagagggaaactaatag

SEQ ID NO: 109 - DNA, 18 bp, synthetic construct
  misc_feature 1..18: note = Description of Artificial Sequence: Synthetic primer
  source 1..18: mol_type = other DNA
ctgatggggagaatgtga

SEQ ID NO: 110 - DNA, 20 bp, synthetic construct
  misc_feature 1..20: note = Description of Artificial Sequence: Synthetic primer
  source 1..20: mol_type = other DNA
catttgaccatgaccatgta

SEQ ID NO: 111 - DNA, 20 bp, synthetic construct
  misc_feature 1..20: note = Description of Artificial Sequence: Synthetic primer
  source 1..20: mol_type = other DNA
cccatgaataccagtgacta

SEQ ID NO: 112 - DNA, 20 bp, synthetic construct
  misc_feature 1..20: note = Description of Artificial Sequence: Synthetic primer
  source 1..20: mol_type = other DNA
ctgttacttacgtggacatt

SEQ ID NO: 113 - DNA, 19 bp, synthetic construct
  misc_feature 1..19: note = Description of Artificial Sequence: Synthetic primer
  source 1..19: mol_type = other DNA
ctcattcatcgccacatag

SEQ ID NO: 114 - DNA, 20 bp, synthetic construct
  misc_feature 1..20: note = Description of Artificial Sequence: Synthetic primer
  source 1..20: mol_type = other DNA
gacttaccttgcaatgtttg

SEQ ID NO: 115 - DNA, 18 bp, synthetic construct
  misc_feature 1..18: note = Description of Artificial Sequence: Synthetic primer
  source 1..18: mol_type = other DNA
actggcctgtctcaatat

SEQ ID NO: 116 - DNA, 20 bp, synthetic construct
  misc_feature 1..20: note = Description of Artificial Sequence: Synthetic primer
  source 1..20: mol_type = other DNA
agtggaagtatgcccatata

SEQ ID NO: 117 - DNA, 21 bp, synthetic construct
  misc_feature 1..21: note = Description of Artificial Sequence: Synthetic primer
  source 1..21: mol_type = other DNA
tacagaagaggagtgtcatat

SEQ ID NO: 118 - DNA, 16 bp, synthetic construct
  misc_feature 1..16: note = Description of Artificial Sequence: Synthetic primer
  source 1..16: mol_type = other DNA
tgcccactgtgttact

SEQ ID NO: 119 - DNA, 21 bp, synthetic construct
  misc_feature 1..21: note = Description of Artificial Sequence: Synthetic primer
  source 1..21: mol_type = other DNA
agttgtgggtacctttagatt

SEQ ID NO: 120 - DNA, 12 bp, synthetic construct
  misc_feature 1..12: note = Description of Artificial Sequence: Synthetic primer
  source 1..12: mol_type = other DNA
ggcggtggtggt

SEQ ID NO: 121 - DNA, 14 bp, synthetic construct
  misc_feature 1..14: note = Description of Artificial Sequence: Synthetic primer
  source 1..14: mol_type = other DNA
gccaggcctcaacg

SEQ ID NO: 122 - DNA, 15 bp, synthetic construct
  misc_feature 1..15: note = Description of Artificial Sequence: Synthetic primer
  source 1..15: mol_type = other DNA
gtgaccgaggacaac

SEQ ID NO: 123 - DNA, 15 bp, synthetic construct
  misc_feature 1..15: note = Description of Artificial Sequence: Synthetic primer
  source 1..15: mol_type = other DNA
ctgaccgacgttgac

SEQ ID NO: 124 - DNA, 17 bp, synthetic construct
  misc_feature 1..17: note = Description of Artificial Sequence: Synthetic primer
  source 1..17: mol_type = other DNA
cctgggattgcagattg

SEQ ID NO: 125 - DNA, 20 bp, synthetic construct
  misc_feature 1..20: note = Description of Artificial Sequence: Synthetic primer
  source 1..20: mol_type = other DNA
gtttcatggactcagttact

SEQ ID NO: 126 - DNA, 20 bp, synthetic construct
  misc_feature 1..20: note = Description of Artificial Sequence: Synthetic primer
  source 1..20: mol_type = other DNA
ccagacctttgctttagatt

SEQ ID NO: 127 - DNA, 20 bp, synthetic construct
  misc_feature 1..20: note = Description of Artificial Sequence: Synthetic primer
  source 1..20: mol_type = other DNA
aaagacttggtgttgttgat

SEQ ID NO: 128 - DNA, 20 bp, synthetic construct
  misc_feature 1..20: note = Description of Artificial Sequence: Synthetic primer
  source 1..20: mol_type = other DNA
gcccatcatatttcttcaga

SEQ ID NO: 129 - DNA, 17 bp, synthetic construct
  misc_feature 1..17: note = Description of Artificial Sequence: Synthetic primer
  source 1..17: mol_type = other DNA
cgtgccacccagaatat

SEQ ID NO: 130 - DNA, 14 bp, synthetic construct
  misc_feature 1..14: note = Description of Artificial Sequence: Synthetic primer
  source 1..14: mol_type = other DNA
ggcctacctggtcg

SEQ ID NO: 131 - DNA, 19 bp, synthetic construct
  misc_feature 1..19: note = Description of Artificial Sequence: Synthetic primer
  source 1..19: mol_type = other DNA
ttccagcactctgacatat

SEQ ID NO: 132 - DNA, 16 bp, synthetic construct
  misc_feature 1..16: note = Description of Artificial Sequence: Synthetic primer
  source 1..16: mol_type = other DNA
tgcttttagggcccac

SEQ ID NO: 133 - DNA, 22 bp, synthetic construct
  misc_feature 1..22: note = Description of Artificial Sequence: Synthetic primer
  source 1..22: mol_type = other DNA
ccaacctaatagtgtattcaca

SEQ ID NO: 134 - DNA, 20 bp, synthetic construct
  misc_feature 1..20: note = Description of Artificial Sequence: Synthetic primer
  source 1..20: mol_type = other DNA
attattgactctgttgtgct

SEQ ID NO: 135 - DNA, 17 bp, synthetic construct
  misc_feature 1..17: note = Description of Artificial Sequence: Synthetic primer
  source 1..17: mol_type = other DNA
ccccagtcctcatgtac

SEQ ID NO: 136 - DNA, 16 bp, synthetic construct
  misc_feature 1..16: note = Description of Artificial Sequence: Synthetic primer
  source 1..16: mol_type = other DNA
gctggaggagctagag

SEQ ID NO: 137 - DNA, 20 bp, synthetic construct
  misc_feature 1..20: note = Description of Artificial Sequence: Synthetic primer
  source 1..20: mol_type = other DNA
ccttctcttccccaatctac

SEQ ID NO: 138 - DNA, 16 bp, synthetic construct
  misc_feature 1..16: note = Description of Artificial Sequence: Synthetic primer
  source 1..16: mol_type = other DNA
aggagccaggcatttt

SEQ ID NO: 139 - DNA, 20 bp, synthetic construct
  misc_feature 1..20: note = Description of Artificial Sequence: Synthetic primer
  source 1..20: mol_type = other DNA
ctttccccacaatcatactg

SEQ ID NO: 140 - DNA, 19 bp, synthetic construct
  misc_feature 1..19: note = Description of Artificial Sequence: Synthetic primer
  source 1..19: mol_type = other DNA
gctggtgttgtctcaatat

SEQ ID NO: 141 - DNA, 19 bp, synthetic construct
  misc_feature 1..19: note = Description of Artificial Sequence: Synthetic primer
  source 1..19: mol_type = other DNA
tacgatgcaagagtacaca

SEQ ID NO: 142 - DNA, 18 bp, synthetic construct
  misc_feature 1..18: note = Description of Artificial Sequence: Synthetic primer
  source 1..18: mol_type = other DNA
ccaccactggatttctca

SEQ ID NO: 143 - DNA, 20 bp, synthetic construct
  misc_feature 1..20: note = Description of Artificial Sequence: Synthetic primer
  source 1..20: mol_type = other DNA
cttattggctttggtcttca

SEQ ID NO: 144 - DNA, 27 bp, synthetic construct
  misc_feature 1..27: note = Description of Artificial Sequence: Synthetic primer
  source 1..27: mol_type = other DNA
tcagcatcattgtaaattattctattt

SEQ ID NO: 145 - DNA, 15 bp, synthetic construct
  misc_feature 1..15: note = Description of Artificial Sequence: Synthetic primer
  source 1..15: mol_type = other DNA
acgagctggaccact

SEQ ID NO: 146 - DNA, 15 bp, synthetic construct
  misc_feature 1..15: note = Description of Artificial Sequence: Synthetic primer
  source 1..15: mol_type = other DNA
tcggctctccactca

SEQ ID NO: 147 - DNA, 16 bp, synthetic construct
  misc_feature 1..16: note = Description of Artificial Sequence: Synthetic primer
  source 1..16: mol_type = other DNA
gggccacacttactct

SEQ ID NO: 148 - DNA, 14 bp, synthetic construct
  misc_feature 1..14: note = Description of Artificial Sequence: Synthetic primer
  source 1..14: mol_type = other DNA
ccgggtctcactca

SEQ ID NO: 149 - DNA, 20 bp, synthetic construct
  misc_feature 1..20: note = Description of Artificial Sequence: Synthetic primer
  source 1..20: mol_type = other DNA
cgtagtaggggaagatcatc

SEQ ID NO: 150 - DNA, 17 bp, synthetic construct
  misc_feature 1..17: note = Description of Artificial Sequence: Synthetic primer
  source 1..17: mol_type = other DNA
ggacactcgcagtagaa

SEQ ID NO: 151 - DNA, 22 bp, synthetic construct
  misc_feature 1..22: note = Description of Artificial Sequence: Synthetic primer
  source 1..22: mol_type = other DNA
ataatgctcctagtacctgtag

SEQ ID NO: 152 - DNA, 19 bp, synthetic construct
  misc_feature 1..19: note = Description of Artificial Sequence: Synthetic primer
  source 1..19: mol_type = other DNA
cttgcacaaatgctgaaag

SEQ ID NO: 153 - DNA, 23 bp, synthetic construct
  misc_feature 1..23: note = Description of Artificial Sequence: Synthetic primer
  source 1..23: mol_type = other DNA
catttatagaaaccgaggtatga

SEQ ID NO: 154 - DNA, 18 bp, synthetic construct
  misc_feature 1..18: note = Description of Artificial Sequence: Synthetic primer
  source 1..18: mol_type = other DNA
gtctgcaggacaattcat

SEQ ID NO: 155 - DNA, 20 bp, synthetic construct
  misc_feature 1..20: note = Description of Artificial Sequence: Synthetic primer
  source 1..20: mol_type = other DNA
tccacaaagccccttataat

SEQ ID NO: 156 - DNA, 19 bp, synthetic construct
  misc_feature 1..19: note = Description of Artificial Sequence: Synthetic primer
  source 1..19: mol_type = other DNA
cttgtttcaggcatgtagt

SEQ ID NO: 157 - DNA, 14 bp, synthetic construct
  misc_feature 1..14: note = Description of Artificial Sequence: Synthetic primer
  source 1..14: mol_type = other DNA
cccacgccgtctta

SEQ ID NO: 158 - DNA, 16 bp, synthetic construct
  misc_feature 1..16: note = Description of Artificial Sequence: Synthetic primer
  source 1..16: mol_type = other DNA
agcggtggtgtagtac

SEQ ID NO: 159 - DNA, 17 bp, synthetic construct
  misc_feature 1..17: note = Description of Artificial Sequence: Synthetic primer
  source 1..17: mol_type = other DNA
aagtgccttagcagaga

SEQ ID NO: 160 - DNA, 20 bp, synthetic construct
  misc_feature 1..20: note = Description of Artificial Sequence: Synthetic primer
  source 1..20: mol_type = other DNA
agacatcagaaagcatgatc

SEQ ID NO: 161 - DNA, 17 bp, synthetic construct
  misc_feature 1..17: note = Description of Artificial Sequence: Synthetic primer
  source 1..17: mol_type = other DNA
ccatgaggcagagcata

SEQ ID NO: 162 - DNA, 14 bp, synthetic construct
  misc_feature 1..14: note = Description of Artificial Sequence: Synthetic primer
  source 1..14: mol_type = other DNA
ggccatggcctgac

SEQ ID NO: 163 - DNA, 17 bp, synthetic construct
  misc_feature 1..17: note = Description of Artificial Sequence: Synthetic primer
  source 1..17: mol_type = other DNA
tatggtcatggaagggg

SEQ ID NO: 164 - DNA, 13 bp, synthetic construct
  misc_feature 1..13: note = Description of Artificial Sequence: Synthetic primer
  source 1..13: mol_type = other DNA
ccgctgagccact

SEQ ID NO: 165 - DNA, 19 bp, synthetic construct
  misc_feature 1..19: note = Description of Artificial Sequence: Synthetic primer
  source 1..19: mol_type = other DNA
gcgtcatcatctttgtcat

SEQ ID NO: 166 - DNA, 18 bp, synthetic construct
  misc_feature 1..18: note = Description of Artificial Sequence: Synthetic primer
  source 1..18: mol_type = other DNA
catccctgactgtgagat

SEQ ID NO: 167 - DNA, 15 bp, synthetic construct
  misc_feature 1..15: note = Description of Artificial Sequence: Synthetic primer
  source 1..15: mol_type = other DNA
ggtggcatggacaga

SEQ ID NO: 168 - DNA, 18 bp, synthetic construct
  misc_feature 1..18: note = Description of Artificial Sequence: Synthetic primer
  source 1..18: mol_type = other DNA
gtgggctacaagaactac

SEQ ID NO: 169 - DNA, 18 bp, synthetic construct
  misc_feature 1..18: note = Description of Artificial Sequence: Synthetic primer
  source 1..18: mol_type = other DNA
gggtatggacacgttcat

SEQ ID NO: 170 - DNA, 20 bp, synthetic construct
  misc_feature 1..20: note = Description of Artificial Sequence: Synthetic primer
  source 1..20: mol_type = other DNA
cctcgaaatgaagctacaac

SEQ ID NO: 171 - DNA, 19 bp, synthetic construct
  misc_feature 1..19: note = Description of Artificial Sequence: Synthetic primer
  source 1..19: mol_type = other DNA
aagtggagaatgtcagtct

SEQ ID NO: 172 - DNA, 22 bp, synthetic construct
  misc_feature 1..22: note = Description of Artificial Sequence: Synthetic primer
  source 1..22: mol_type = other DNA
gaaactttccacttgataagag

SEQ ID NO: 173 - DNA, 17 bp, synthetic construct
  misc_feature 1..17: note = Description of Artificial Sequence: Synthetic primer
  source 1..17: mol_type = other DNA
ttacctcgcttagtgct

SEQ ID NO: 174 - DNA, 15 bp, synthetic construct
  misc_feature 1..15: note = Description of Artificial Sequence: Synthetic primer
  source 1..15: mol_type = other DNA
cccagagaccccagt

SEQ ID NO: 175 - DNA, 15 bp, synthetic construct
  misc_feature 1..15: note = Description of Artificial Sequence: Synthetic primer
  source 1..15: mol_type = other DNA
agccctgtcgtctct

SEQ ID NO: 176 - DNA, 16 bp, synthetic construct
  misc_feature 1..16: note = Description of Artificial Sequence: Synthetic primer
  source 1..16: mol_type = other DNA
gccaggcattgaagtc

SEQ ID NO: 177 - DNA, 14 bp, synthetic construct
  misc_feature 1..14: note = Description of Artificial Sequence: Synthetic primer
  source 1..14: mol_type = other DNA
gagctgctggtgca

SEQ ID NO: 178 - DNA, 18 bp, synthetic construct
  misc_feature 1..18: note = Description of Artificial Sequence: Synthetic primer
  source 1..18: mol_type = other DNA
tccaatggatccactcac

SEQ ID NO: 179 - DNA, 19 bp, synthetic construct
  misc_feature 1..19: note = Description of Artificial Sequence: Synthetic primer
  source 1..19: mol_type = other DNA
gcatttcctttcttcccag

SEQ ID NO: 180 - DNA, 19 bp, synthetic construct
  misc_feature 1..19: note = Description of Artificial Sequence: Synthetic primer
  source 1..19: mol_type = other DNA
ttggttacatccctctctg

SEQ ID NO: 181 - DNA, 16 bp, synthetic construct
  misc_feature 1..16: note = Description of Artificial Sequence: Synthetic primer
  source 1..16: mol_type = other DNA
tgttgcagctgaccac

SEQ ID NO: 182 - DNA, 23 bp, synthetic construct
  misc_feature 1..23: note = Description of Artificial Sequence: Synthetic primer
  source 1..23: mol_type = other DNA
tgttttcctttacttactacacc

SEQ ID NO: 183 - DNA, 20 bp, synthetic construct
  misc_feature 1..20: note = Description of Artificial Sequence: Synthetic primer
  source 1..20: mol_type = other DNA
tttggcttgacttgactttt

SEQ ID NO: 184 - DNA, 21 bp, synthetic construct
  misc_feature 1..21: note = Description of Artificial Sequence: Synthetic primer
  source 1..21: mol_type = other DNA
tgaaatgacacttggagtaac

SEQ ID NO: 185 - DNA, 19 bp, synthetic construct
  misc_feature 1..19: note = Description of Artificial Sequence: Synthetic primer
  source 1..19: mol_type = other DNA
gtatccacatcctcttcct

SEQ ID NO: 186 - DNA, 20 bp, synthetic construct
  misc_feature 1..20: note = Description of Artificial Sequence: Synthetic primer
  source 1..20: mol_type = other DNA
tggtcagtacaagcacatac

SEQ ID NO: 187 - DNA, 20 bp, synthetic construct
  misc_feature 1..20: note = Description of Artificial Sequence: Synthetic primer
  source 1..20: mol_type = other DNA
atgggctttcttgatgtaac

SEQ ID NO: 188 - DNA, 16 bp, synthetic construct
  misc_feature 1..16: note = Description of Artificial Sequence: Synthetic primer
  source 1..16: mol_type = other DNA
ggttccgccaagagat

SEQ ID NO: 189 - DNA, 21 bp, synthetic construct
  misc_feature 1..21: note = Description of Artificial Sequence: Synthetic primer
  source 1..21: mol_type = other DNA
ggccaatttcccattctaata

SEQ ID NO: 190 - DNA, 16 bp, synthetic construct
  misc_feature 1..16: note = Description of Artificial Sequence: Synthetic primer
  source 1..16: mol_type = other DNA
tgacaaaccgagcact

SEQ ID NO: 191 - DNA, 19 bp, synthetic construct
  misc_feature 1..19: note = Description of Artificial Sequence: Synthetic primer
  source 1..19: mol_type = other DNA
cttcatcccccagtaagtc

SEQ ID NO: 192 - DNA, 20 bp, synthetic construct
  misc_feature 1..20: note = Description of Artificial Sequence: Synthetic primer
  source 1..20: mol_type = other DNA
ttctgggagttttcgtatca

SEQ ID NO: 193 - DNA, 17 bp, synthetic construct
  misc_feature 1..17: note = Description of Artificial Sequence: Synthetic primer
  source 1..17: mol_type = other DNA
tccacagccacatcttt

SEQ ID NO: 194 - DNA, 17 bp, synthetic construct
  misc_feature 1..17: note = Description of Artificial Sequence: Synthetic primer
  source 1..17: mol_type = other DNA
ctgacagtgcgtacatc

SEQ ID NO: 195 - DNA, 21 bp, synthetic construct
  misc_feature 1..21: note = Description of Artificial Sequence: Synthetic primer
  source 1..21: mol_type = other DNA
tcattccaaagtcagctatct

SEQ ID NO: 196 - DNA, 19 bp, synthetic construct
  misc_feature 1..19: note = Description of Artificial Sequence: Synthetic primer
  source 1..19: mol_type = other DNA
aggtccacatccattcatc

SEQ ID NO: 197 - DNA, 17 bp, synthetic construct
  misc_feature 1..17: note = Description of Artificial Sequence: Synthetic primer
  source 1..17: mol_type = other DNA
cataggcatgggtgagt

SEQ ID NO: 198 - DNA, 21 bp, synthetic construct
  misc_feature 1..21: note = Description of Artificial Sequence: Synthetic primer
  source 1..21: mol_type = other DNA
ccttcagtccggttttatttg

SEQ ID NO: 199 - DNA, 19 bp, synthetic construct
  misc_feature 1..19: note = Description of Artificial Sequence: Synthetic primer
  source 1..19: mol_type = other DNA
cagaaagcggtgacttact

SEQ ID NO: 200 - DNA, 17 bp, synthetic construct
  misc_feature 1..17: note = Description of Artificial Sequence: Synthetic primer
  source 1..17: mol_type = other DNA
gctaatggcccgttctc

SEQ ID NO: 201 - DNA, 17 bp, synthetic construct
  misc_feature 1..17: note = Description of Artificial Sequence: Synthetic primer
  source 1..17: mol_type = other DNA
cccaccagaccatgaga

SEQ ID NO: 202 - DNA, 20 bp, synthetic construct
  misc_feature 1..20: note = Description of Artificial Sequence: Synthetic primer
  source 1..20: mol_type = other DNA
agccaatattgtctttgtgt

SEQ ID NO: 203 - DNA, 20 bp, synthetic construct
  misc_feature 1..20: note = Description of Artificial Sequence: Synthetic primer
  source 1..20: mol_type = other DNA
ctccttctgcatggtattct

SEQ ID NO: 204 - DNA, 22 bp, synthetic construct
  misc_feature 1..22: note = Description of Artificial Sequence: Synthetic primer
  source 1..22: mol_type = other DNA
cattagcatcaggattatgact

SEQ ID NO: 205 - DNA, 16 bp, synthetic construct
  misc_feature 1..16: note = Description of Artificial Sequence: Synthetic primer
  source 1..16: mol_type = other DNA
ccagagcccagacctg

SEQ ID NO: 206 - DNA, 23 bp, synthetic construct
  misc_feature 1..23: note = Description of Artificial Sequence: Synthetic primer
  source 1..23: mol_type = other DNA
ggagttactatatgggaactgat

SEQ ID NO: 207 - DNA, 22 bp, synthetic construct
  misc_feature 1..22: note = Description of Artificial Sequence: Synthetic primer
  source 1..22: mol_type = other DNA
aactctgagtcttgtttctaca

SEQ ID NO: 208 - DNA, 20 bp, synthetic construct
  misc_feature 1..20: note = Description of Artificial Sequence: Synthetic primer
  source 1..20: mol_type = other DNA
cctgcatgaatttcaatgac

SEQ ID NO: 209 - DNA, 20 bp, synthetic construct
  misc_feature 1..20: note = Description of Artificial Sequence: Synthetic primer
  source 1..20: mol_type = other DNA
gttatgcagacaccattcat

SEQ ID NO: 210 - DNA, 20 bp, synthetic construct
  misc_feature 1..20: note = Description of Artificial Sequence: Synthetic primer
  source 1..20: mol_type = other DNA
aattgcattcacacgttaac

SEQ ID NO: 211 - DNA, 23 bp, synthetic construct
  misc_feature 1..23: note = Description of Artificial Sequence: Synthetic primer
  source 1..23: mol_type = other DNA
ccttgactaaatctaccatgttt

SEQ ID NO: 212 - DNA, 21 bp, synthetic construct
  misc_feature 1..21: note = Description of Artificial Sequence: Synthetic primer
  source 1..21: mol_type = other DNA
ccagtgtctgagaacattagt

SEQ ID NO: 213 - DNA, 20 bp, synthetic construct
  misc_feature 1..20: note = Description of Artificial Sequence: Synthetic primer
  source 1..20: mol_type = other DNA
agacagcacagaattgatac

SEQ ID NO: 214 - DNA, 23 bp, synthetic construct
  misc_feature 1..23: note = Description of Artificial Sequence: Synthetic primer
  source 1..23: mol_type = other DNA
tcagtgtattcatcgagatttag

SEQ ID NO: 215 - DNA, 19 bp, synthetic construct
  misc_feature 1..19: note = Description of Artificial Sequence: Synthetic primer
  source 1..19: mol_type = other DNA
tgtctgcaaggtttacagt

SEQ ID NO: 216 - DNA, 16 bp, synthetic construct
  misc_feature 1..16: note = Description of Artificial Sequence: Synthetic primer
  source 1..16: mol_type = other DNA
gctgtgcgtcactgta

SEQ ID NO: 217 - DNA, 13 bp, synthetic construct
  misc_feature 1..13: note = Description of Artificial Sequence: Synthetic primer
  source 1..13: mol_type = other DNA
cgcaggcggcaga

SEQ ID NO: 218 - DNA, 14 bp, synthetic construct
  misc_feature 1..14: note = Description of Artificial Sequence: Synthetic primer
  source 1..14: mol_type = other DNA
ggaaggcggtgttg

SEQ ID NO: 219 - DNA, 14 bp, synthetic construct
  misc_feature 1..14: note = Description of Artificial Sequence: Synthetic primer
  source 1..14: mol_type = other DNA
gcctgctgtggccc

SEQ ID NO: 220 - DNA, 15 bp, synthetic construct
  misc_feature 1..15: note = Description of Artificial Sequence: Synthetic primer
  source 1..15: mol_type = other DNA
agggcgacgagaaac

SEQ ID NO: 221 - DNA, 22 bp, synthetic construct
  misc_feature 1..22: note = Description of Artificial Sequence: Synthetic primer
  source 1..22: mol_type = other DNA
tcctctagctatcttaatgact

SEQ ID NO: 222 - DNA, 20 bp, synthetic construct
  misc_feature 1..20: note = Description of Artificial Sequence: Synthetic primer
  source 1..20: mol_type = other DNA
gcttactggaagttgacttt

SEQ ID NO: 223 - DNA, 17 bp, synthetic construct
  misc_feature 1..17: note = Description of Artificial Sequence: Synthetic primer
  source 1..17: mol_type = other DNA
ggaagcaggtggtcatt

SEQ ID NO: 224 - DNA, 18 bp, synthetic construct
  misc_feature 1..18: note = Description of Artificial Sequence: Synthetic primer
  source 1..18: mol_type = other DNA
tggcttctcctctacaga

SEQ ID NO: 225 - DNA, 20 bp, synthetic construct
  misc_feature 1..20: note = Description of Artificial Sequence: Synthetic primer
  source 1..20: mol_type = other DNA
ggatgctgcagaagctataa

SEQ ID NO: 226 - DNA, 20 bp, synthetic construct
  misc_feature 1..20: note = Description of Artificial Sequence: Synthetic primer
  source 1..20: mol_type = other DNA
gttcaagctgaagaagatgt

SEQ ID NO: 227 - DNA, 18 bp, synthetic construct
  misc_feature 1..18: note = Description of Artificial Sequence: Synthetic primer
  source 1..18: mol_type = other DNA
gctcagttcctggacaaa

SEQ ID NO: 228 - DNA, 22 bp, synthetic construct
  misc_feature 1..22: note = Description of Artificial Sequence: Synthetic primer
  source 1..22: mol_type = other DNA
cctttgaatgcagaagattctt

SEQ ID NO: 229 - DNA, 18 bp, synthetic construct
  misc_feature 1..18: note = Description of Artificial Sequence: Synthetic primer
  source 1..18: mol_type = other DNA
agcagagaatgggtactc

SEQ ID NO: 230 - DNA, 18 bp, synthetic construct
  misc_feature 1..18: note = Description of Artificial Sequence: Synthetic primer
  source 1..18: mol_type = other DNA
gttgtctttggcaaggat

SEQ ID NO: 231 - DNA, 21 bp, synthetic construct
  misc_feature 1..21: note = Description of Artificial Sequence: Synthetic primer
  source 1..21: mol_type = other DNA
gcactgtaataatccagactg

SEQ ID NO: 232 - DNA, 17 bp, synthetic construct
  misc_feature 1..17: note = Description of Artificial Sequence: Synthetic primer
  source 1..17: mol_type = other DNA
gtgggagaccttgaaca

SEQ ID NO: 233 - DNA, 17 bp, synthetic construct
  misc_feature 1..17: note = Description of Artificial Sequence: Synthetic primer
  source 1..17: mol_type = other DNA
gccatggagtcgatgag

SEQ ID NO: 234 - DNA, 19 bp, synthetic construct
  misc_feature 1..19: note = Description of Artificial Sequence: Synthetic primer
  source 1..19: mol_type = other DNA
gcctgacaaatccagagta

SEQ ID NO: 235 - DNA, 19 bp, synthetic construct
  misc_feature 1..19: note = Description of Artificial Sequence: Synthetic primer
  source 1..19: mol_type = other DNA
tgatgaaccggtcctttac

SEQ ID NO: 236 - DNA, 21 bp, synthetic construct
  misc_feature 1..21: note = Description of Artificial Sequence: Synthetic primer
  source 1..21: mol_type = other DNA
cgacgacaatcttaaactgta

SEQ ID NO: 237 - DNA, 20 bp, synthetic construct
  misc_feature 1..20: note = Description of Artificial Sequence: Synthetic primer
  source 1..20: mol_type = other DNA
acaacccactgaggtatatg

SEQ ID NO: 238 - DNA, 19 bp, synthetic construct
  misc_feature 1..19: note = Description of Artificial Sequence: Synthetic primer
  source 1..19: mol_type = other DNA
tgtatggtaggaccaccag

SEQ ID NO: 239 - DNA, 20 bp, synthetic construct
  misc_feature 1..20: note = Description of Artificial Sequence: Synthetic primer
  source 1..20: mol_type = other DNA
aaacagattcctccttgtca

SEQ ID NO: 240 - DNA, 19 bp, synthetic construct
  misc_feature 1..19: note = Description of Artificial Sequence: Synthetic primer
  source 1..19: mol_type = other DNA
gcacctgttttgttgtgta

SEQ ID NO: 241 - DNA, 14 bp, synthetic construct
  misc_feature 1..14: note = Description of Artificial Sequence: Synthetic primer
  source 1..14: mol_type = other DNA
cccacccgtgaccg

SEQ ID NO: 242 - DNA, 16 bp, synthetic construct
  misc_feature 1..16: note = Description of Artificial Sequence: Synthetic primer
  source 1..16: mol_type = other DNA
caggtgcagccacaaa

SEQ ID NO: 243 - DNA, 20 bp, synthetic construct
  misc_feature 1..20: note = Description of Artificial Sequence: Synthetic primer
  source 1..20: mol_type = other DNA
tctacctggagattgacaac

SEQ ID NO: 244 - DNA, 16 bp, synthetic construct
  misc_feature 1..16: note = Description of Artificial Sequence: Synthetic primer
  source 1..16: mol_type = other DNA
caagcaccccatcaag

SEQ ID NO: 245 - DNA, 13 bp, synthetic construct
  misc_feature 1..13: note = Description of Artificial Sequence: Synthetic primer
  source 1..13: mol_type = other DNA
ccggcacgctggt

SEQ ID NO: 246 - DNA, 15 bp, synthetic construct
  misc_feature 1..15: note = Description of Artificial Sequence: Synthetic primer
  source 1..15: mol_type = other DNA
ccacggtggctacaa

SEQ ID NO: 247 - DNA, 20 bp, synthetic construct
  misc_feature 1..20: note = Description of Artificial Sequence: Synthetic primer
  source 1..20: mol_type = other DNA
acctgtttgttggacatact

SEQ ID NO: 248 - DNA, 19 bp, synthetic construct
  misc_feature 1..19: note = Description of Artificial Sequence: Synthetic primer
  source 1..19: mol_type = other DNA
aagactcggatgatgtacc

SEQ ID NO: 249 - DNA, 19 bp, synthetic construct
  misc_feature 1..19: note = Description of Artificial Sequence: Synthetic primer
  source 1..19: mol_type = other DNA
ggttaccccatggaactta

SEQ ID NO: 250 - DNA, 20 bp, synthetic construct
  misc_feature 1..20: note = Description of Artificial Sequence: Synthetic primer
  source 1..20: mol_type = other DNA
caggaagctatccctattct

SEQ ID NO: 251 - DNA, 17 bp, synthetic construct
  misc_feature 1..17: note = Description of Artificial Sequence: Synthetic primer
  source 1..17: mol_type = other DNA
gttaccccaacggctac

SEQ ID NO: 252 - DNA, 20 bp, synthetic construct
  misc_feature 1..20: note = Description of Artificial Sequence: Synthetic primer
  source 1..20: mol_type = other DNA
tgcagatggcatcattaatc

SEQ ID NO: 253 - DNA, 18 bp, synthetic construct
  misc_feature 1..18: note = Description of Artificial Sequence: Synthetic primer
  source 1..18: mol_type = other DNA
cacccagaaagcagacta

SEQ ID NO: 254 - DNA, 18 bp, synthetic construct
  misc_feature 1..18: note = Description of Artificial Sequence: Synthetic primer
  source 1..18: mol_type = other DNA
cgacacacacgacaatac

SEQ ID NO: 255 - DNA, 18 bp, synthetic construct
  misc_feature 1..18: note = Description of Artificial Sequence: Synthetic primer
  source 1..18: mol_type = other DNA
gtcacagccttcttcatg

SEQ ID NO: 256 - DNA, 15 bp, synthetic construct
  misc_feature 1..15: note = Description of Artificial Sequence: Synthetic primer
  source 1..15: mol_type = other DNA
cgcatggcctcttct

SEQ ID NO: 257 - DNA, 16 bp, synthetic construct
  misc_feature 1..16: note = Description of Artificial Sequence: Synthetic primer
  source 1..16: mol_type = other DNA
gcaccgagacgatgaa

SEQ ID NO: 258 - DNA, 15 bp, synthetic construct
  misc_feature 1..15: note = Description of Artificial Sequence: Synthetic primer
  source 1..15: mol_type = other DNA
gcacctggctcctct

SEQ ID NO: 259 - DNA, 20 bp, synthetic construct
  misc_feature 1..20: note = Description of Artificial Sequence: Synthetic primer
  source 1..20: mol_type = other DNA
ccccatacaatttgatgaca

SEQ ID NO: 260 - DNA, 14 bp, synthetic construct
  misc_feature 1..14: note = Description of Artificial Sequence: Synthetic primer
  source 1..14: mol_type = other DNA
tccgccgcacttac

SEQ ID NO: 261 - DNA, 18 bp, synthetic construct
  misc_feature 1..18: note = Description of Artificial Sequence: Synthetic primer
  source 1..18: mol_type = other DNA
gtgagcaggtggaagtag

SEQ ID NO: 262 - DNA, 15 bp, synthetic construct
  misc_feature 1..15: note = Description of Artificial Sequence: Synthetic primer
  source 1..15: mol_type = other DNA
ccagatgagcagcgt

SEQ ID NO: 263 - DNA, 20 bp, synthetic construct
  misc_feature 1..20: note = Description of Artificial Sequence: Synthetic primer
  source 1..20: mol_type = other DNA
tcttcctcctcttcttcttc

SEQ ID NO: 264 - DNA, 16 bp, synthetic construct
  misc_feature 1..16: note = Description of Artificial Sequence: Synthetic primer
  source 1..16: mol_type = other DNA
cagcaagtgcccagta

SEQ ID NO: 265 - DNA, 16 bp, synthetic construct
  misc_feature 1..16: note = Description of Artificial Sequence: Synthetic primer
  source 1..16: mol_type = other DNA
ccgagtccagcacctc

SEQ ID NO: 266 - DNA, 16 bp, synthetic construct
  misc_feature 1..16: note = Description of Artificial Sequence: Synthetic primer
  source 1..16: mol_type = other DNA
aggctcccacctttca

SEQ ID NO: 267 - DNA, 18 bp, synthetic construct
  misc_feature 1..18: note = Description of Artificial Sequence: Synthetic primer
  source 1..18: mol_type = other DNA
acagaccctctcactcat

SEQ ID NO: 268 - DNA, 17 bp, synthetic construct
  misc_feature 1..17: note = Description of Artificial Sequence: Synthetic primer
  source 1..17: mol_type = other DNA
ccaagggtgcagttatg

SEQ ID NO: 269 - DNA, 17 bp, synthetic construct
  misc_feature 1..17: note = Description of Artificial Sequence: Synthetic primer
  source 1..17: mol_type = other DNA
tgcctcttgcttctctt

SEQ ID NO: 270 - DNA, 20 bp, synthetic construct
  misc_feature 1..20: note = Description of Artificial Sequence: Synthetic primer
  source 1..20: mol_type = other DNA
cctcactgattgctcttagg

SEQ ID NO: 271 - DNA, 16 bp, synthetic construct
  misc_feature 1..16: note = Description of Artificial Sequence: Synthetic primer
  source 1..16: mol_type = other DNA
gcagctgtgggttgat

SEQ ID NO: 272 - DNA, 16 bp, synthetic construct
  misc_feature 1..16: note = Description of Artificial Sequence: Synthetic primer
  source 1..16: mol_type = other DNA
ggcccctgtcatcttc

SEQ ID NO: 273 - DNA, 18 bp, synthetic construct
  misc_feature 1..18: note = Description of Artificial Sequence: Synthetic primer
  source 1..18: mol_type = other DNA
gtcctctgactgctcttt

SEQ ID NO: 274 - DNA, 17 bp, synthetic construct
  misc_feature 1..17: note = Description of Artificial Sequence: Synthetic primer
  source 1..17: mol_type = other DNA
gctggatccccactttt

SEQ ID NO: 275 - DNA, 17 bp, synthetic construct
  misc_feature 1..17: note = Description of Artificial Sequence: Synthetic primer
  source 1..17: mol_type = other DNA
tcacgttggtccacatc

SEQ ID NO: 276 - DNA, 18 bp, synthetic construct
  misc_feature 1..18: note = Description of Artificial Sequence: Synthetic primer
  source 1..18: mol_type = other DNA
acaggatgacaggaagag

SEQ ID NO: 277 - DNA, 20 bp, synthetic construct
  misc_feature 1..20: note = Description of Artificial Sequence: Synthetic primer
  source 1..20: mol_type = other DNA
acgatttcccttggagatat

SEQ ID NO: 278 - DNA, 20 bp, synthetic construct
  misc_feature 1..20: note = Description of Artificial Sequence: Synthetic primer
  source 1..20: mol_type = other DNA
agacaactgttcaaactgat

SEQ ID NO: 279 - DNA, 20 bp, synthetic construct
  misc_feature 1..20: note = Description of Artificial Sequence: Synthetic primer
  source 1..20: mol_type = other DNA
gaccccaagctttagtaaat

SEQ ID NO: 280 - DNA, 19 bp, synthetic construct
  misc_feature 1..19: note = Description of Artificial Sequence: Synthetic primer
  source 1..19: mol_type = other DNA
acaccctccattttatcac

SEQ ID NO: 281 - DNA, 20 bp, synthetic construct
  misc_feature 1..20: note = Description of Artificial Sequence: Synthetic primer
  source 1..20: mol_type = other DNA
cacgaatgtgtggttaactc

SEQ ID NO: 282 - DNA, 17 bp, synthetic construct
  misc_feature 1..17: note = Description of Artificial Sequence: Synthetic primer
  source 1..17: mol_type = other DNA
tttccttcccccttgtc

SEQ ID NO: 283 - DNA, 20 bp, synthetic construct
  misc_feature 1..20: note = Description of Artificial Sequence: Synthetic primer
  source 1..20: mol_type = other DNA
ttgtattctctgccttctct

SEQ ID NO: 284 - DNA, 18 bp, synthetic construct
  misc_feature 1..18: note = Description of Artificial Sequence: Synthetic primer
  source 1..18: mol_type = other DNA
aataaccgctcctcatca

SEQ ID NO: 285 - DNA, 22 bp, synthetic construct
  misc_feature 1..22: note = Description of Artificial Sequence: Synthetic primer
  source 1..22: mol_type = other DNA
ctgagactagatgacttttgtc

SEQ ID NO: 286 - DNA, 23 bp, synthetic construct
  misc_feature 1..23: note = Description of Artificial Sequence: Synthetic primer
  source 1..23: mol_type = other DNA
ctttacttaaatagggcaagttc

SEQ ID NO: 287 - DNA, 23 bp, synthetic construct
  misc_feature 1..23: note = Description of Artificial Sequence: Synthetic primer
  source 1..23: mol_type = other DNA
ttcctttatttttgttcccaaag

SEQ ID NO: 288 - DNA, 15 bp, synthetic construct
  misc_feature 1..15: note = Description of Artificial Sequence: Synthetic primer
  source 1..15: mol_type = other DNA
caacgccacaaccac

SEQ ID NO: 289 - DNA, 19 bp, synthetic construct
  misc_feature 1..19: note = Description of Artificial Sequence: Synthetic primer
  source 1..19: mol_type = other DNA
gtaccagatggatgtgaac

SEQ ID NO: 290 - DNA, 20 bp, synthetic construct
  misc_feature 1..20: note = Description of Artificial Sequence: Synthetic primer
  source 1..20: mol_type = other DNA
agagacatgcatgaacattt

SEQ ID NO: 291 - DNA, 19 bp, synthetic construct
  misc_feature 1..19: note = Description of Artificial Sequence: Synthetic primer
  source 1..19: mol_type = other DNA
cttgtctctgtgttcttgt

SEQ ID NO: 292 - DNA, 20 bp, synthetic construct
  misc_feature 1..20: note = Description of Artificial Sequence: Synthetic primer
  source 1..20: mol_type = other DNA
agttaacgtcttccttctct

SEQ ID NO: 293 - DNA, 15 bp, synthetic construct
  misc_feature 1..15: note = Description of Artificial Sequence: Synthetic primer
  source 1..15: mol_type = other DNA
gcatctgcctcacct

SEQ ID NO: 294 - DNA, 16 bp, synthetic construct
  misc_feature 1..16: note = Description of Artificial Sequence: Synthetic primer
  source 1..16: mol_type = other DNA
agccaggaacgtactg

SEQ ID NO: 295 - DNA, 20 bp, synthetic construct
  misc_feature 1..20: note = Description of Artificial Sequence: Synthetic primer
  source 1..20: mol_type = other DNA
aagggattgtgattgttcat

SEQ ID NO: 296 - DNA, 20 bp, synthetic construct
  misc_feature 1..20: note = Description of Artificial Sequence: Synthetic primer
  source 1..20: mol_type = other DNA
cctgatctccttagacaact

SEQ ID NO: 297 - DNA, 17 bp, synthetic construct
  misc_feature 1..17: note = Description of Artificial Sequence: Synthetic primer
  source 1..17: mol_type = other DNA
ataccctctcagcgtac

SEQ ID NO: 298 - DNA, 16 bp, synthetic construct
  misc_feature 1..16: note = Description of Artificial Sequence: Synthetic primer
  source 1..16: mol_type = other DNA
ggggatgagctacctg

SEQ ID NO: 299 - DNA, 16 bp, synthetic construct
  misc_feature 1..16: note = Description of Artificial Sequence: Synthetic primer
  source 1..16: mol_type = other DNA
ttggccagcaagaatg

SEQ ID NO: 300 - DNA, 18 bp, synthetic construct
  misc_feature 1..18: note = Description of Artificial Sequence: Synthetic primer
  source 1..18: mol_type = other DNA
cagtgcaaggtttacaca

SEQ ID NO: 301 - DNA, 21 bp, synthetic construct
  misc_feature 1..21: note = Description of Artificial Sequence: Synthetic primer
  source 1..21: mol_type = other DNA
gaaaggagagcaggataataa

SEQ ID NO: 302 - DNA, 24 bp, synthetic construct
  misc_feature 1..24: note = Description of Artificial Sequence: Synthetic primer
  source 1..24: mol_type = other DNA
cactgatatttaaatgccttagag

SEQ ID NO: 303 - DNA, 18 bp, synthetic construct
  misc_feature 1..18: note = Description of Artificial Sequence: Synthetic primer
  source 1..18: mol_type = other DNA
cagcagcttggtttcttc

SEQ ID NO: 304 - DNA, 17 bp, synthetic construct
  misc_feature 1..17: note = Description of Artificial Sequence: Synthetic primer
  source 1..17: mol_type = other DNA
tgcaacgtgtgtagaca

SEQ ID NO: 305 - DNA, 17 bp, synthetic construct
  misc_feature 1..17: note = Description of Artificial Sequence: Synthetic primer
  source 1..17: mol_type = other DNA
tcccaaccatgacaaga

SEQ ID NO: 306 - DNA, 21 bp, synthetic construct
  misc_feature 1..21: note = Description of Artificial Sequence: Synthetic primer
  source 1..21: mol_type = other DNA
gctacttgcaatgatatacac

SEQ ID NO: 307 - DNA, 22 bp, synthetic construct
  misc_feature 1..22: note = Description of Artificial Sequence: Synthetic primer
  source 1..22: mol_type = other DNA
cacccaatgaagaatgtaattg

SEQ ID NO: 308 - DNA, 17 bp, synthetic construct
  misc_feature 1..17: note = Description of Artificial Sequence: Synthetic primer
  source 1..17: mol_type = other DNA
agcccagccatttctaa

SEQ ID NO: 309 - DNA, 16 bp, synthetic construct
  misc_feature 1..16: note = Description of Artificial Sequence: Synthetic primer
  source 1..16: mol_type = other DNA
ctggctgctgaagtct

SEQ ID NO: 310 - DNA, 17 bp, synthetic construct
  misc_feature 1..17: note = Description of Artificial Sequence: Synthetic primer
  source 1..17: mol_type = other DNA
cactgtggaggcatttg

SEQ ID NO: 311 - DNA, 15 bp, synthetic construct
  misc_feature 1..15: note = Description of Artificial Sequence: Synthetic primer
  source 1..15: mol_type = other DNA
cagtggctcaagcac

SEQ ID NO: 312 - DNA, 14 bp, synthetic construct
  misc_feature 1..14: note = Description of Artificial Sequence: Synthetic primer
  source 1..14: mol_type = other DNA
gaggagcccgtgtc

SEQ ID NO: 313 - DNA, 15 bp, synthetic construct
  misc_feature 1..15: note = Description of Artificial Sequence: Synthetic primer
  source 1..15: mol_type = other DNA
ctgcagctggtcctt

SEQ ID NO: 314 - DNA, 16 bp, synthetic construct
  misc_feature 1..16: note = Description of Artificial Sequence: Synthetic primer
  source 1..16: mol_type = other DNA
gcaggagctgacagta

SEQ ID NO: 315 - DNA, 17 bp, synthetic construct
  misc_feature 1..17: note = Description of Artificial Sequence: Synthetic primer
  source 1..17: mol_type = other DNA
cggtagttgcccttctc

SEQ ID NO: 316 - DNA, 16 bp, synthetic construct
  misc_feature 1..16: note = Description of Artificial Sequence: Synthetic primer
  source 1..16: mol_type = other DNA
cgctgtgtcctttcag

SEQ ID NO: 317 - DNA, 22 bp, synthetic construct
  misc_feature 1..22: note = Description of Artificial Sequence: Synthetic primer
  source 1..22: mol_type = other DNA
gacattttcaaagcagtgtatc

SEQ ID NO: 318 - DNA, 18 bp, synthetic construct
  misc_feature 1..18: note = Description of Artificial Sequence: Synthetic primer
  source 1..18: mol_type = other DNA
ccagtccctctggaataa

SEQ ID NO: 319 - DNA, 19 bp, synthetic construct
  misc_feature 1..19: note = Description of Artificial Sequence: Synthetic primer
  source 1..19: mol_type = other DNA
ctctatagtggggtcgtat

SEQ ID NO: 320 - DNA, 20 bp, synthetic construct
  misc_feature 1..20: note = Description of Artificial Sequence: Synthetic primer
  source 1..20: mol_type = other DNA
acattattgccaacatgact

SEQ ID NO: 321 - DNA, 20 bp, synthetic construct
  misc_feature 1..20: note = Description of Artificial Sequence: Synthetic primer
  source 1..20: mol_type = other DNA
catactcaacacgattctgt

SEQ ID NO: 322 - DNA, 17 bp, synthetic construct
  misc_feature 1..17: note = Description of Artificial Sequence: Synthetic primer
  source 1..17: mol_type = other DNA
ggcacggttgaatgtaa

SEQ ID NO: 323 - DNA, 21 bp, synthetic construct
  misc_feature 1..21: note = Description of Artificial Sequence: Synthetic primer
  source 1..21: mol_type = other DNA
ccacatttctcttccattgta

SEQ ID NO: 324 - DNA, 29 bp, synthetic construct
  misc_feature 1..29: note = Description of Artificial Sequence: Synthetic primer
  source 1..29: mol_type = other DNA
agacaataattattaaaaggtgatctatt

SEQ ID NO: 325 - DNA, 16 bp, synthetic construct
  misc_feature 1..16: note = Description of Artificial Sequence: Synthetic primer
  source 1..16: mol_type = other DNA
ttagcgagtgcccatt

SEQ ID NO: 326 - DNA, 18 bp, synthetic construct
  misc_feature 1..18: note = Description of Artificial Sequence: Synthetic primer
  source 1..18: mol_type = other DNA
tgtcccacttgattcagt

SEQ ID NO: 327 - DNA, 18 bp, synthetic construct
  misc_feature 1..18: note = Description of Artificial Sequence: Synthetic primer
  source 1..18: mol_type = other DNA
cgtgagtacccattctct

SEQ ID NO: 328 - DNA, 22 bp, synthetic construct
  misc_feature 1..22: note = Description of Artificial Sequence: Synthetic primer
  source 1..22: mol_type = other DNA
ccaaagacaacttcattagact

SEQ ID NO: 329 - DNA, 26 bp, synthetic construct
  misc_feature 1..26: note = Description of Artificial Sequence: Synthetic primer
  source 1..26: mol_type = other DNA
gtatttatttcagtgttacttacctg

SEQ ID NO: 330 - DNA, 20 bp, synthetic construct
  misc_feature 1..20: note = Description of Artificial Sequence: Synthetic primer
  source 1..20: mol_type = other DNA
ggatcatattcgtccacaaa

SEQ ID NO: 331 - DNA, 21 bp, synthetic construct
  misc_feature 1..21: note = Description of Artificial Sequence: Synthetic primer
  source 1..21: mol_type = other DNA
ctttctccagctaattcatct

SEQ ID NO: 332 - DNA, 17 bp, synthetic construct
  misc_feature 1..17: note = Description of Artificial Sequence: Synthetic primer
  source 1..17: mol_type = other DNA
ctgcagccagaaagact

SEQ ID NO: 333 - DNA, 19 bp, synthetic construct
  misc_feature 1..19: note = Description of Artificial Sequence: Synthetic primer
  source 1..19: mol_type = other DNA
tcctctcgtttccttacat

SEQ ID NO: 334 - DNA, 18 bp, synthetic construct
  misc_feature 1..18: note = Description of Artificial Sequence: Synthetic primer
  source 1..18: mol_type = other DNA
caaagcaagccagattct

SEQ ID NO: 335 - DNA, 17 bp, synthetic construct
  misc_feature 1..17: note = Description of Artificial Sequence: Synthetic primer
  source 1..17: mol_type = other DNA
gcactgggtcaaagtct

SEQ ID NO: 336 - DNA, 19 bp, synthetic construct
  misc_feature 1..19: note = Description of Artificial Sequence: Synthetic primer
  source 1..19: mol_type = other DNA
cagtgctaaccaagttctt

SEQ ID NO: 337 - DNA, 15 bp, synthetic construct
  misc_feature 1..15: note = Description of Artificial Sequence: Synthetic primer
  source 1..15: mol_type = other DNA
cgctcctgggaatct

SEQ ID NO: 338 - DNA, 18 bp, synthetic construct
  misc_feature 1..18: note = Description of Artificial Sequence: Synthetic primer
  source 1..18: mol_type = other DNA
cagtcaaggttgctgatt

SEQ ID NO: 339 - DNA, 14 bp, synthetic construct
  misc_feature 1..14: note = Description of Artificial Sequence: Synthetic primer
  source 1..14: mol_type = other DNA
ggcgtcaggaactg

SEQ ID NO: 340 - DNA, 16 bp, synthetic construct
  misc_feature 1..16: note = Description of Artificial Sequence: Synthetic primer
  source 1..16: mol_type = other DNA
cggcctcgatcttgta

SEQ ID NO: 341 - DNA, 14 bp, synthetic construct
  misc_feature 1..14: note = Description of Artificial Sequence: Synthetic primer
  source 1..14: mol_type = other DNA
cagctcctcctcgc

SEQ ID NO: 342 - DNA, 21 bp, synthetic construct
  misc_feature 1..21: note = Description of Artificial Sequence: Synthetic primer
  source 1..21: mol_type = other DNA
ggtgggatcatattcatctac

SEQ ID NO: 343 - DNA, 22 bp, synthetic construct
  misc_feature 1..22: note = Description of Artificial Sequence: Synthetic primer
  source 1..22: mol_type = other DNA
gtctgaactgaagataatgact

SEQ ID NO: 344 - DNA, 16 bp, synthetic construct
  misc_feature 1..16: note = Description of Artificial Sequence: Synthetic primer
  source 1..16: mol_type = other DNA
tgtccaccgtgatctg

SEQ ID NO: 345 - DNA, 19 bp, synthetic construct
  misc_feature 1..19: note = Description of Artificial Sequence: Synthetic primer
  source 1..19: mol_type = other DNA
tcgtctgggaactatactc

SEQ ID NO: 346 - DNA, 18 bp, synthetic construct
  misc_feature 1..18: note = Description of Artificial Sequence: Synthetic primer
  source 1..18: mol_type = other DNA
tgtactgctcccagaaga

SEQ ID NO: 347 - DNA, 17 bp, synthetic construct
  misc_feature 1..17: note = Description of Artificial Sequence: Synthetic primer
  source 1..17: mol_type = other DNA
tgcaccaggagtttgta

SEQ ID NO: 348 - DNA, 15 bp, synthetic construct
  misc_feature 1..15: note = Description of Artificial Sequence: Synthetic primer
  source 1..15: mol_type = other DNA
gagggtgtcctgtgt

SEQ ID NO: 349 - DNA, 17 bp, synthetic construct
  misc_feature 1..17: note = Description of Artificial Sequence: Synthetic primer
  source 1..17: mol_type = other DNA
gccacagcagtctgaaa

SEQ ID NO: 350 - DNA, 19 bp, synthetic construct
  misc_feature 1..19: note = Description of Artificial Sequence: Synthetic primer
  source 1..19: mol_type = other DNA
aggttcgctgcttttaatc

SEQ ID NO: 351 - DNA, 16 bp, synthetic construct
  misc_feature 1..16: note = Description of Artificial Sequence: Synthetic primer
  source 1..16: mol_type = other DNA
cccgggggattaaagc

SEQ ID NO: 352 - DNA, 18 bp, synthetic construct
  misc_feature 1..18: note = Description of Artificial Sequence: Synthetic primer
  source 1..18: mol_type = other DNA
ggtcagctactcctcttc

SEQ ID NO: 353 - DNA, 17 bp, synthetic construct
  misc_feature 1..17: note = Description of Artificial Sequence: Synthetic primer
  source 1..17: mol_type = other DNA
cccctccttcctagaga

SEQ ID NO: 354 - DNA, 18 bp, synthetic construct
  misc_feature 1..18: note = Description of Artificial Sequence: Synthetic primer
  source 1..18: mol_type = other DNA
gtgctgcatttcagagaa

SEQ ID NO: 355 - DNA, 19 bp, synthetic construct
  misc_feature 1..19: note = Description of Artificial Sequence: Synthetic primer
  source 1..19: mol_type = other DNA
gtgcagaacatcaagttca

SEQ ID NO: 356 - DNA, 18 bp, synthetic construct
  misc_feature 1..18: note = Description of Artificial Sequence: Synthetic primer
  source 1..18: mol_type = other DNA
tggctttgtgctcattac

SEQ ID NO: 357 - DNA, 17 bp, synthetic construct
  misc_feature 1..17: note = Description of Artificial Sequence: Synthetic primer
  source 1..17: mol_type = other DNA
ctgctctcaggttgact

SEQ ID NO: 358 - DNA, 15 bp, synthetic construct
  misc_feature 1..15: note = Description of Artificial Sequence: Synthetic primer
  source 1..15: mol_type = other DNA
gagctggggactctt

SEQ ID NO: 359 - DNA, 19 bp, synthetic construct
  misc_feature 1..19: note = Description of Artificial Sequence: Synthetic primer
  source 1..19: mol_type = other DNA
caggagtcatgactctgtt

SEQ ID NO: 360 - DNA, 17 bp, synthetic construct
  misc_feature 1..17: note = Description of Artificial Sequence: Synthetic primer
  source 1..17: mol_type = other DNA
tggcctttgacctcaat

SEQ ID NO: 361 - DNA, 14 bp, synthetic construct
  misc_feature 1..14: note = Description of Artificial Sequence: Synthetic primer
  source 1..14: mol_type = other DNA
gcccgcaggtactt

SEQ ID NO: 362 - DNA, 15 bp, synthetic construct
  misc_feature 1..15: note = Description of Artificial Sequence: Synthetic primer
  source 1..15: mol_type = other DNA
atcctccggctgaag

SEQ ID NO: 363 - DNA, 15 bp, synthetic construct
  misc_feature 1..15: note = Description of Artificial Sequence: Synthetic primer
  source 1..15: mol_type = other DNA
ctggagtgagccctg

SEQ ID NO: 364 - DNA, 17 bp, synthetic construct
  misc_feature 1..17: note = Description of Artificial Sequence: Synthetic primer
  source 1..17: mol_type = other DNA
gcagtgctaggaaagag

SEQ ID NO: 365 - DNA, 13 bp, synthetic construct
  misc_feature 1..13: note = Description of Artificial Sequence: Synthetic primer
  source 1..13: mol_type = other DNA
tgcagggtggcaa

SEQ ID NO: 366 - DNA, 16 bp, synthetic construct
  misc_feature 1..16: note = Description of Artificial Sequence: Synthetic primer
  source 1..16: mol_type = other DNA
cgtcatgtgctgtgac

SEQ ID NO: 367 - DNA, 21 bp, synthetic construct
  misc_feature 1..21: note = Description of Artificial Sequence: Synthetic primer
  source 1..21: mol_type = other DNA
gtgtttctgtcatccaaatac

SEQ ID NO: 368 - DNA, 17 bp, synthetic construct
  misc_feature 1..17: note = Description of Artificial Sequence: Synthetic primer
  source 1..17: mol_type = other DNA
tgccctggtaggttttc

SEQ ID NO: 369 - DNA, 17 bp, synthetic construct
  misc_feature 1..17: note = Description of Artificial Sequence: Synthetic primer
  source 1..17: mol_type = other DNA
caagggggactgtagat

SEQ ID NO: 370 - DNA, 17 bp, synthetic construct
  misc_feature 1..17: note = Description of Artificial Sequence: Synthetic primer
  source 1..17: mol_type = other DNA
ggtctgacgggtagagt

SEQ ID NO: 371 - DNA, 17 bp, synthetic construct
  misc_feature 1..17: note = Description of Artificial Sequence: Synthetic primer
  source 1..17: mol_type = other DNA
tctgtctccccacagag

SEQ ID NO: 372 - DNA, 18 bp, synthetic construct
  misc_feature 1..18: note = Description of Artificial Sequence: Synthetic primer
  source 1..18: mol_type = other DNA
tgaagtgtgctctgaaca

SEQ ID NO: 373 - DNA, 18 bp, synthetic construct
  misc_feature 1..18: note = Description of Artificial Sequence: Synthetic primer
  source 1..18: mol_type = other DNA
atgcttgctctgatagga

SEQ ID NO: 374 - DNA, 23 bp, synthetic construct
  misc_feature 1..23: note = Description of Artificial Sequence: Synthetic primer
  source 1..23: mol_type = other DNA
acacaagactcagaatagataca

SEQ ID NO: 375 - DNA, 20 bp, synthetic construct
  misc_feature 1..20: note = Description of Artificial Sequence: Synthetic primer
  source 1..20: mol_type = other DNA
atgtctcttcttcctacctg

SEQ ID NO: 376 - DNA, 17 bp, synthetic construct
  misc_feature 1..17: note = Description of Artificial Sequence: Synthetic primer
  source 1..17: mol_type = other DNA
gccacacgtggtattca

SEQ ID NO: 377 - DNA, 18 bp, synthetic construct
  misc_feature 1..18: note = Description of Artificial Sequence: Synthetic primer
  source 1..18: mol_type = other DNA
tgagctccctacctgatt

SEQ ID NO: 378 - DNA, 19 bp, synthetic construct
  misc_feature 1..19: note = Description of Artificial Sequence: Synthetic primer
  source 1..19: mol_type = other DNA
caccttctccagcattttc

SEQ ID NO: 379 - DNA, 19 bp, synthetic construct
  misc_feature 1..19: note = Description of Artificial Sequence: Synthetic primer
  source 1..19: mol_type = other DNA
catcatcctcacctgactc

SEQ ID NO: 380 - DNA, 21 bp, synthetic construct
  misc_feature 1..21: note = Description of Artificial Sequence: Synthetic primer
  source 1..21: mol_type = other DNA
ttcaaaatgtagaccacagac

SEQ ID NO: 381 - DNA, 21 bp, synthetic construct
  misc_feature 1..21: note = Description of Artificial Sequence: Synthetic primer
  source 1..21: mol_type = other DNA
cggaagaactctccagtattc

SEQ ID NO: 382 - DNA, 19 bp, synthetic construct
  misc_feature 1..19: note = Description of Artificial Sequence: Synthetic primer
  source 1..19: mol_type = other DNA
ccttgttgaaggagcagaa

SEQ ID NO: 383 - DNA, 15 bp, synthetic construct
  misc_feature 1..15: note = Description of Artificial Sequence: Synthetic primer
  source 1..15: mol_type = other DNA
ccagagcccgactcg

SEQ ID NO: 384 - DNA, 19 bp, synthetic construct
  misc_feature 1..19: note = Description of Artificial Sequence: Synthetic primer
  source 1..19: mol_type = other DNA
tctccaagatgggatactc

SEQ ID NO: 385 - DNA, 16 bp, synthetic construct
  misc_feature 1..16: note = Description of Artificial Sequence: Synthetic primer
  source 1..16: mol_type = other DNA
ggcgtctgcgtacttc

SEQ ID NO: 386 - DNA, 20 bp, synthetic construct
  misc_feature 1..20: note = Description of Artificial Sequence: Synthetic primer
  source 1..20: mol_type = other DNA
cagggaccttaccttataca

SEQ ID NO: 387 - DNA, 15 bp, synthetic construct
  misc_feature 1..15: note = Description of Artificial Sequence: Synthetic primer
  source 1..15: mol_type = other DNA
cccccacacagcaaa

SEQ ID NO: 388 - DNA, 21 bp, synthetic construct
  misc_feature 1..21: note = Description of Artificial Sequence: Synthetic primer
  source 1..21: mol_type = other DNA
gtatctcccttccctgattac

SEQ ID NO: 389 - DNA, 15 bp, synthetic construct
  misc_feature 1..15: note = Description of Artificial Sequence: Synthetic primer
  source 1..15: mol_type = other DNA
tggtccctggtgtca

SEQ ID NO: 390 - DNA, 17 bp, synthetic construct
  misc_feature 1..17: note = Description of Artificial Sequence: Synthetic primer
  source 1..17: mol_type = other DNA
aggcgttctcctttctc

SEQ ID NO: 391 - DNA, 18 bp, synthetic construct
  misc_feature 1..18: note = Description of Artificial Sequence: Synthetic primer
  source 1..18: mol_type = other DNA
gcagtgagtgggtacctc

SEQ ID NO: 392 - DNA, 18 bp, synthetic construct
  misc_feature 1..18: note = Description of Artificial Sequence: Synthetic primer
  source 1..18: mol_type = other DNA
ccggacatggtctaagag

SEQ ID NO: 393 - DNA, 18 bp, synthetic construct
  misc_feature 1..18: note = Description of Artificial Sequence: Synthetic primer
  source 1..18: mol_type = other DNA
ccatctgcatggtactct

SEQ ID NO: 394 - DNA, 21 bp, synthetic construct
  misc_feature 1..21: note = Description of Artificial Sequence: Synthetic primer
  source 1..21: mol_type = other DNA
gtgctctcattttaaagatgg

SEQ ID NO: 395 - DNA, 21 bp, synthetic construct
  misc_feature 1..21: note = Description of Artificial Sequence: Synthetic primer
  source 1..21: mol_type = other DNA
gcttgtttgctgaatgttaac

SEQ ID NO: 396 - DNA, 18 bp, synthetic construct
  misc_feature 1..18: note = Description of Artificial Sequence: Synthetic primer
  source 1..18: mol_type = other DNA
gtgtgcagaacaatgtga

SEQ ID NO: 397 - DNA, 21 bp, synthetic construct
  misc_feature 1..21: note = Description of Artificial Sequence: Synthetic primer
  source 1..21: mol_type = other DNA
ggacttcaagaacttggatta

SEQ ID NO: 398 - DNA, 20 bp, synthetic construct
  misc_feature 1..20: note = Description of Artificial Sequence: Synthetic primer
  source 1..20: mol_type = other DNA
actatgggacttgaaaacgg

SEQ ID NO: 399 - DNA, 18 bp, synthetic construct
  misc_feature 1..18: note = Description of Artificial Sequence: Synthetic primer
  source 1..18: mol_type = other DNA
acaggccagtgtttacat

SEQ ID NO: 400 - DNA, 23 bp, synthetic construct
  misc_feature 1..23: note = Description of Artificial Sequence: Synthetic primer
  source 1..23: mol_type = other DNA
aacatcatcattagtggatctac

SEQ ID NO: 401 - DNA, 20 bp, synthetic construct
  misc_feature 1..20: note = Description of Artificial Sequence: Synthetic primer
  source 1..20: mol_type = other DNA
tgtgatcacatgcttacagt

SEQ ID NO: 402 - DNA, 22 bp, synthetic construct
  misc_feature 1..22: note = Description of Artificial Sequence: Synthetic primer
  source 1..22: mol_type = other DNA
gagaaattgcttgctttagatg

SEQ ID NO: 403 - DNA, 20 bp, synthetic construct
  misc_feature 1..20: note = Description of Artificial Sequence: Synthetic primer
  source 1..20: mol_type = other DNA
gtgtcagagatggagatgat

SEQ ID NO: 404 - DNA, 15 bp, synthetic construct
  misc_feature 1..15: note = Description of Artificial Sequence: Synthetic primer
  source 1..15: mol_type = other DNA
agcgcctggaagaga

SEQ ID NO: 405 - DNA, 17 bp, synthetic construct
  misc_feature 1..17: note = Description of Artificial Sequence: Synthetic primer
  source 1..17: mol_type = other DNA
ggcatgaggtcactgac

SEQ ID NO: 406 - DNA, 16 bp, synthetic construct
  misc_feature 1..16: note = Description of Artificial Sequence: Synthetic primer
  source 1..16: mol_type = other DNA
gctccaacccctagac

SEQ ID NO: 407 - DNA, 17 bp, synthetic construct
  misc_feature 1..17: note = Description of Artificial Sequence: Synthetic primer
  source 1..17: mol_type = other DNA
acaccaggtccttgaag

SEQ ID NO: 408 - DNA, 16 bp, synthetic construct
  misc_feature 1..16: note = Description of Artificial Sequence: Synthetic primer
  source 1..16: mol_type = other DNA
gggatgccactcacag

SEQ ID NO: 409 - DNA, 18 bp, synthetic construct
  misc_feature 1..18: note = Description of Artificial Sequence: Synthetic primer
  source 1..18: mol_type = other DNA
ggaagctggcaatctcta

SEQ ID NO: 410 - DNA, 21 bp, synthetic construct
  misc_feature 1..21: note = Description of Artificial Sequence: Synthetic primer
  source 1..21: mol_type = other DNA
cccgttctacgagaagaataa

SEQ ID NO: 411 - DNA, 14 bp, synthetic construct
  misc_feature 1..14: note = Description of Artificial Sequence: Synthetic primer
  source 1..14: mol_type = other DNA
gcccacctcgttgt

SEQ ID NO: 412 - DNA, 21 bp, synthetic construct
  misc_feature 1..21: note = Description of Artificial Sequence: Synthetic primer
  source 1..21: mol_type = other DNA
ccctaagtttgtaagtagtgc

SEQ ID NO: 413 - DNA, 20 bp, synthetic construct
  misc_feature 1..20: note = Description of Artificial Sequence: Synthetic primer
  source 1..20: mol_type = other DNA
agaagcaaagcgttctttac

SEQ ID NO: 414 - DNA, 17 bp, synthetic construct
  misc_feature 1..17: note = Description of Artificial Sequence: Synthetic primer
  source 1..17: mol_type = other DNA
agaccctgtaggaggac

SEQ ID NO: 415 - DNA, 20 bp, synthetic construct
  misc_feature 1..20: note = Description of Artificial Sequence: Synthetic primer
  source 1..20: mol_type = other DNA
tggcaccatacgaaatattc

SEQ ID NO: 416 - DNA, 18 bp, synthetic construct
  misc_feature 1..18: note = Description of Artificial Sequence: Synthetic primer
  source 1..18: mol_type = other DNA
ccggatcagtgcataaca

SEQ ID NO: 417 - DNA, 22 bp, synthetic construct
  misc_feature 1..22: note = Description of Artificial Sequence: Synthetic primer
  source 1..22: mol_type = other DNA
atcatgactgatatggtagaca

SEQ ID NO: 418 - DNA, 23 bp, synthetic construct
  misc_feature 1..23: note = Description of Artificial Sequence: Synthetic primer
  source 1..23: mol_type = other DNA
tctggagagagaacaaataaatg

SEQ ID NO: 419 - DNA, 17 bp, synthetic construct
  misc_feature 1..17: note = Description of Artificial Sequence: Synthetic primer
  source 1..17: mol_type = other DNA
actcagcctgtttctgg

SEQ ID NO: 420 - DNA, 20 bp, synthetic construct
  misc_feature 1..20: note = Description of Artificial Sequence: Synthetic primer
  source 1..20: mol_type = other DNA
cctgacagacaataaaaggc

SEQ ID NO: 421 - DNA, 20 bp, synthetic construct
  misc_feature 1..20: note = Description of Artificial Sequence: Synthetic primer
  source 1..20: mol_type = other DNA
gcttgataggtaggtactca

SEQ ID NO: 422 - DNA, 20 bp, synthetic construct
  misc_feature 1..20: note = Description of Artificial Sequence: Synthetic primer
  source 1..20: mol_type = other DNA
catgcaaattttgctgaagt

SEQ ID NO: 423 - DNA, 22 bp, synthetic construct
  misc_feature 1..22: note = Description of Artificial Sequence: Synthetic primer
  source 1..22: mol_type = other DNA
ccttccttgatcatcttgtaga

SEQ ID NO: 424 - DNA, 21 bp, synthetic construct
  misc_feature 1..21: note = Description of Artificial Sequence: Synthetic primer
  source 1..21: mol_type = other DNA
agttaaggactctgaagatgt

SEQ ID NO: 425 - DNA, 24 bp, synthetic construct
  misc_feature 1..24: note = Description of Artificial Sequence: Synthetic primer
  source 1..24: mol_type = other DNA
gtgtgacatgttctaatatagtca

SEQ ID NO: 426 - DNA, 18 bp, synthetic construct
  misc_feature 1..18: note = Description of Artificial Sequence: Synthetic primer
  source 1..18: mol_type = other DNA
gctccatgcagatactga

SEQ ID NO: 427 - DNA, 16 bp, synthetic construct
  misc_feature 1..16: note = Description of Artificial Sequence: Synthetic primer
  source 1..16: mol_type = other DNA
cctggcacccaaacat

SEQ ID NO: 428 - DNA, 19 bp, synthetic construct
  misc_feature 1..19: note = Description of Artificial Sequence: Synthetic primer
  source 1..19: mol_type = other DNA
gcttcccaaacacttagac

SEQ ID NO: 429 - DNA, 18 bp, synthetic construct
  misc_feature 1..18: note = Description of Artificial Sequence: Synthetic primer
  source 1..18: mol_type = other DNA
aagcagtgctcatgattg

SEQ ID NO: 430 - DNA, 17 bp, synthetic construct
  misc_feature 1..17: note = Description of Artificial Sequence: Synthetic primer
  source 1..17: mol_type = other DNA
ttgggcttacacttcgg

SEQ ID NO: 431 - DNA, 22 bp, synthetic construct
  misc_feature 1..22: note = Description of Artificial Sequence: Synthetic primer
  source 1..22: mol_type = other DNA
aaccacaaaagtatactccatg

SEQ ID NO: 432 - DNA, 16 bp, synthetic construct
  misc_feature 1..16: note = Description of Artificial Sequence: Synthetic primer
  source 1..16: mol_type = other DNA
ggatggctggcttaca

SEQ ID NO: 433 - DNA, 20 bp, synthetic construct
  misc_feature 1..20: note = Description of Artificial Sequence: Synthetic primer
  source 1..20: mol_type = other DNA
catctgacttggtggtaaac

SEQ ID NO: 434 - DNA, 16 bp, synthetic construct
  misc_feature 1..16: note = Description of Artificial Sequence: Synthetic primer
  source 1..16: mol_type = other DNA
cggagcttcctgagtg

SEQ ID NO: 435 - DNA, 17 bp, synthetic construct
  misc_feature 1..17: note = Description of Artificial Sequence: Synthetic primer
  source 1..17: mol_type = other DNA
gcccctctctgattgtc

SEQ ID NO: 436 - DNA, 16 bp, synthetic construct
  misc_feature 1..16: note = Description of Artificial Sequence: Synthetic primer
  source 1..16: mol_type = other DNA
ggtggtggtgctgatg

SEQ ID NO: 437 - DNA, 19 bp, synthetic construct
  misc_feature 1..19: note = Description of Artificial Sequence: Synthetic primer
  source 1..19: mol_type = other DNA
cgccaattaaccctgatta

SEQ ID NO: 438 - DNA, 21 bp, synthetic construct
  misc_feature 1..21: note = Description of Artificial Sequence: Synthetic primer
  source 1..21: mol_type = other DNA
ggatttaagcctgattgaaca

SEQ ID NO: 439 - DNA, 17 bp, synthetic construct
  misc_feature 1..17: note = Description of Artificial Sequence: Synthetic primer
  source 1..17: mol_type = other DNA
accagtgagggaagtga

SEQ ID NO: 440 - DNA, 20 bp, synthetic construct
  misc_feature 1..20: note = Description of Artificial Sequence: Synthetic primer
  source 1..20: mol_type = other DNA
ggtaaatggacaagaacact

SEQ ID NO: 441 - DNA, 18 bp, synthetic construct
  misc_feature 1..18: note = Description of Artificial Sequence: Synthetic primer
  source 1..18: mol_type = other DNA
gcccagttccctttctac

SEQ ID NO: 442 - DNA, 19 bp, synthetic construct
  misc_feature 1..19: note = Description of Artificial Sequence: Synthetic primer
  source 1..19: mol_type = other DNA
tctggctgcgagttataat

SEQ ID NO: 443 - DNA, 18 bp, synthetic construct
  misc_feature 1..18: note = Description of Artificial Sequence: Synthetic primer
  source 1..18: mol_type = other DNA
cagctttgcccatgaaac

SEQ ID NO: 444 - DNA, 16 bp, synthetic construct
  misc_feature 1..16: note = Description of Artificial Sequence: Synthetic primer
  source 1..16: mol_type = other DNA
ccaggcagcggtagta

SEQ ID NO: 445 - DNA, 16 bp, synthetic construct
  misc_feature 1..16: note = Description of Artificial Sequence: Synthetic primer
  source 1..16: mol_type = other DNA
gctactggccggaaag

SEQ ID NO: 446 - DNA, 19 bp, synthetic construct
  misc_feature 1..19: note = Description of Artificial Sequence: Synthetic primer
  source 1..19: mol_type = other DNA
tcagatgtgctgttgagac

SEQ ID NO: 447 - DNA, 16 bp, synthetic construct
  misc_feature 1..16: note = Description of Artificial Sequence: Synthetic primer
  source 1..16: mol_type = other DNA
aggatggcctctgtct

SEQ ID NO: 448 - DNA, 18 bp, synthetic construct
  misc_feature 1..18: note = Description of Artificial Sequence: Synthetic primer
  source 1..18: mol_type = other DNA
gagcaacacccacactta

SEQ ID NO: 449 - DNA, 16 bp, synthetic construct
  misc_feature 1..16: note = Description of Artificial Sequence: Synthetic primer
  source 1..16: mol_type = other DNA
cagctggccttaccat

SEQ ID NO: 450 - DNA, 17 bp, synthetic construct
  misc_feature 1..17: note = Description of Artificial Sequence: Synthetic primer
  source 1..17: mol_type = other DNA
gtagctgtgcatgtcct

SEQ ID NO: 451 - DNA, 18 bp, synthetic construct
  misc_feature 1..18: note = Description of Artificial Sequence: Synthetic primer
  source 1..18: mol_type = other DNA
ggaggtgggtgtctttat

SEQ ID NO: 452 - DNA, 16 bp, synthetic construct
  misc_feature 1..16: note = Description of Artificial Sequence: Synthetic primer
  source 1..16: mol_type = other DNA
gtcgtgggacacagtg

SEQ ID NO: 453 - DNA, 17 bp, synthetic construct
  misc_feature 1..17: note = Description of Artificial Sequence: Synthetic primer
  source 1..17: mol_type = other DNA
ggtgcgggagtgaatag

SEQ ID NO: 454 - DNA, 16 bp, synthetic construct
  misc_feature 1..16: note = Description of Artificial Sequence: Synthetic primer
  source 1..16: mol_type = other DNA
ggagggtgcagtgttg

SEQ ID NO: 455 - DNA, 19 bp, synthetic construct
  misc_feature 1..19: note = Description of Artificial Sequence: Synthetic primer
  source 1..19: mol_type = other DNA
ctccagaagcttgaactct

SEQ ID NO: 456 - DNA, 16 bp, synthetic construct
  misc_feature 1..16: note = Description of Artificial Sequence: Synthetic primer
  source 1..16: mol_type = other DNA
ctagcacgtgcctacc

SEQ ID NO: 457 - DNA, 20 bp, synthetic construct
  misc_feature 1..20: note = Description of Artificial Sequence: Synthetic primer
  source 1..20: mol_type = other DNA
cctgagtgtagatgatgtca

SEQ ID NO: 458 - DNA, 23 bp, synthetic construct
  misc_feature 1..23: note = Description of Artificial Sequence: Synthetic primer
  source 1..23: mol_type = other DNA
actcaggtactgtgtatatactt

SEQ ID NO: 459 - DNA, 18 bp, synthetic construct
  misc_feature 1..18: note = Description of Artificial Sequence: Synthetic primer
  source 1..18: mol_type = other DNA
gaagagaatctccgcaag

SEQ ID NO: 460 - DNA, 17 bp, synthetic construct
  misc_feature 1..17: note = Description of Artificial Sequence: Synthetic primer
  source 1..17: mol_type = other DNA
gcactggcctcatcttg

SEQ ID NO: 461 - DNA, 18 bp, synthetic construct
  misc_feature 1..18: note = Description of Artificial Sequence: Synthetic primer
  source 1..18: mol_type = other DNA
gccctgactttcaactct

SEQ ID NO: 462 - DNA, 16 bp, synthetic construct
  misc_feature 1..16: note = Description of Artificial Sequence: Synthetic primer
  source 1..16: mol_type = other DNA
agcgctgctcagatag

SEQ ID NO: 463 - DNA, 17 bp, synthetic construct
  misc_feature 1..17: note = Description of Artificial Sequence: Synthetic primer
  source 1..17: mol_type = other DNA
acccaggtccagatgaa

SEQ ID NO: 464 - DNA, 19 bp, synthetic construct
  misc_feature 1..19: note = Description of Artificial Sequence: Synthetic primer
  source 1..19: mol_type = other DNA
actgactttctgctcttgt